(19) United States
(12) Patent Application Publication
(10) Pub. No.: US 2013/0007927 A1
(43) Pub. Date: Jan. 3, 2013

(54) NOVEL CENTROMERES AND METHODS OF USING THE SAME

(75) Inventors: Gregory P. Copenhaver, Chapel Hill, NC (US); Song Luo, Chicago, IL (US); Jennifer M. Mach, Chicago, IL (US); Daphne Preuss, Chicago, IL (US); Rolando Ramirez, Chicago, IL (US)

(73) Assignee: CHROMATIN, INC., Chicago, IL (US)

(21) Appl. No.: 13/522,891

(22) PCT Filed: Jan. 21, 2011
(86) PCT No.: PCT/US11/22168

§ 371 Date: Sep. 25, 2012

Related U.S. Application Data
(60) Provisional application No. 61/297,636, filed on Jan. 22, 2010.

(51) Int. Cl. C12N 15/11; A01H 5/00; C12Q 1/68; C40B 30/04; C12N 15/82; C12N 5/10

(52) U.S. Cl. 800/314; 536/23.1; 435/320.1; 435/419; 435/6.11; 506/9

(57) ABSTRACT

The invention is generally related to compositions and methods related to novel centromere sequences identified in cotton, and resulting recombinant DNA constructs, such as minichromosomes, made using such sequences. Minichromosomes with novel compositions and structures can be used, for example, to transform plant cells that are in turn used to generate minichromosome-harboring plants. The invention is directed to products of such plants, including oil and textiles. The invention is also directed to novel methods for identifying centromere sequences.

### CROSS REFERENCE TO RELATED APPLICATION

This application claims priority to US Provisional patent application by G. Copenhaver, et al., U.S. Provisional Patent Application Ser. No. 61/297,636, titled, “PLANTS MODIFIED WITH MINICHROMOSOMES,” filed Jan. 22, 2010, which is incorporated by reference herein in its entirety. This application also claims priority to US Provisional patent application by G. Copenhaver, et al., U.S. Provisional Patent Application Ser. No. 61/435,202, file Jan. 21, 2011, which is incorporated by reference herein in its entirety.

### FIELD OF THE INVENTION

The present invention relates to methods for identifying centromeric sequences that are useful, for example, in constructing artificial chromosomes, and cells and organisms comprising such artificial chromosomes as these methods relate to cotton. The present invention also discloses centromeric sequences useful, for example, in constructing artificial chromosomes for use in cotton.

### GOVERNMENT SUPPORT

Not applicable.

### COMPACT DISC FOR SEQUENCE LISTINGS AND TABLES

Not applicable

### INCORPORATION BY REFERENCE OF AN ELECTRONICALLY-SUBMITTED SEQUENCE LISTING

This application incorporates by reference the electronically-submitted sequence listing titled, “018WON1_sequence_listing_ST25—FINAL,” an ASCII-compliant file, created 21 Jan. 2011, and is 593 kB and submitted concurrently with this application.

### BACKGROUND OF THE INVENTION

Agricultural crops have the potential to meet escalating global demands for affordable and sustainable production of food, fuels, fibers, therapeutics, and biomaterials (Herrera, 2004). While integrative plant transformation techniques can often meet these needs by safely introducing novel genes into plant chromosomes, they have limited efficiency and can disrupt the host genome. Typically, biological delivery of DNA carried on an Agrobacterium T-DNA plasmid, or biolistic delivery of small DNA-coated particles is used to transfer and integrate desired genes into a host plant chromosome (Lorence and Verpoorte 2004). Integration at random sites results in unpredictable transgene expression due to position effect variegation, variable copy number from tandem integrations, and frequent loss of gene integrity as a result of unpredictable breakage and end joining (Birch, 1997; Lorence and Verpoorte, 2004). Transgene integration can also result in genetic linkage of the introduced genes to portions of the genome that encode loci that confer undesired phenotypes, adding complexity when the transgenic locus is introgressed into other varieties (Walker et al., 2002; Yin et al., 2004). Recent advances in gene integration technologies have aimed to surmount some of these difficulties. For example, zinc finger-mediated homologous recombination or site-specific recombination could eliminate the unpredictable expression that results from random insertion into the plant genome (Gilbertson, 2003; Kumar et al., 2006). In addition, combining binary T-DNA elements with bacterial artificial chromosome (BAC) technology to produce BiBACs has the potential to introduce larger DNA fragments into the host genome (Hamilton et al., 1996; He et al., 2003). In contrast to these systems, minichromosomes (MCs) remain separate (autonomous) from the host chromosomes, and thus provide an alternative approach with important benefits. Indeed, although precise integration into host chromosomes has long been a routine technique in Saccharomyces cerevisiae, the facile properties of autonomous vectors often make them a preferred choice for numerous applications, including commercial-scale protein production.

The first eukaryotic MCMCs used a simple centromere (CEN) sequence from the budding yeast S. cerevisiae, incorporated into versatile circular CEN and linear yeast artificial chromosome (YAC) vectors (Burke et al., 1987; Clarke and Carbon, 1980). These yeast vectors were used to define a 125-bp DNA fragment sufficient for mitotic and meiotic centromere function (Cottarel, Shero et al. 1989). While circular CEN vectors are most useful for carrying smaller DNA fragments, YAC vectors can carry megabase quantities of DNA and are convenient for manipulating large fragments of DNA (Larin et al., 1991). Similarly, with carrying capacities of hundreds of kb, human artificial chromosomes (HACs) provide advantages over other in vitro-assembled vectors used in human cell transfection (Kuroiwa et al., 2000). HACs containing tandem repeats of a 171-bp alpha satellite sequence can be maintained either as circular or linear, telomere-containing, episomes (Ebersole et al., 2000; Harrington et al., 1997; (keno et al., 1998; Schueler et al., 2001; Tsuduki et al., 2006).

DNA sequences that can form stable MCs are able to recapitulate centromere functions de novo by recruiting essential DNA binding proteins and epigenetic modifications. In human cells, different repetitive DNA (satellite) arrays vary in their ability to efficiently form HACs, based on their monomer sequence, chromosomal origin, array length, higher-order structure, and even vector composition (Grimes et al., 2002; Mejia et al., 2002; Ohzeki et al., 2002; Okamoto et al., 2007). These DNA sequences recruit centromere binding protein A (CENP-A), which substitutes for histone H3 to form centromeric nucleosomes; this protein marks active centromeres in S. cerevisiae (Cse4p), Schizosaccharomyces pombe (Cnp1), Drosophila melanogaster (Cid), Arabidopsis thaliana (HTR12), Zea mays (CENH3), and Homo sapiens (CENP-A) (Malik and Henikoff, 2001; Meluh et al., 1998; Palmer et al., 1987; Takahashi et al., 2000; Talbert et al., 2002; Zhong et al., 2002). CENP-A complexes are maintained through mitosis and meiosis (Schatten et al., 1988), resulting in an epigenetic mark that is important in perpetuating centromere activity. Evidence for this role in centromere maintenance comes from human neocentromeres (Lo et al., 2001), where, at a very low frequency, aberrant ectopic centromeres are nucleated in regions that lack satellite DNA. Once formed, these neocentromeres are efficiently perpetuated. The ability to form centromeres on naked DNA also depends on cell type in mammalian systems; indeed, HAC formation has been most commonly demonstrated in HT1080 fibrosarcoma cells. Yet once established, HACs can be transferred to other mammalian cell types, where they are stably maintained (Suzuki et al., 2006).

Maize centromeres contain repetitive sequences that are similar to those found in mammalian centromeres; for example, analogous to the tandem arrays of alpha satellite found in human centromeres, large tandem arrays of the 156-bp maize CentC satellite bind to CENP-A (Ananiev et al., 1998; Nagaki et al., 2003; Zhong et al., 2002). These satellite arrays are often interrupted by CRM, a centromere-specific retroelement that also binds CENP-A (Zhong et al., 2002). Some maize varieties also have supernumerary B chromosomes with a distinct centromere satellite sequence, ZmBs (Alfenito and Birchler, 1993; Jin et al., 2005). These B chromosomes lack essential genes, and thus have been particularly useful for discerning the relationship between centromere structure and meiotic transmission (Kaszas et al., 2002; Kato et al., 2005; Phelps-Durr and Birchler, 2004). A series of deletion derivatives of natural B chromosomes, derived from an A-B translocation event, showed a strong dependence on centromere size—the smallest functional derivative contained a 110-kb centromere and resulted in a meiotic transmission rate of 5%, yet showed a high stability in mitosis (Phelps-Durr and Birchler 2004). More recently, telomere-mediated chromosomal truncation was used to generate deletion derivatives from both A and B maize chromosomes [40]. Transgenes carried on these derivative chromosomes (or “engineered MCs”) were expressed and meiotic inheritance ranged from 12% to 39% (Yu et al., 2007). While this telomere-truncation approach can deliver both transgenes and sequences that promote site-directed integration, its utility for commercial applications can be limited—most commercial maize hybrids lack B chromosomes.

Carlson et al. (2007) have described autonomous MCs that do not rely on alteration of endogenous chromosomes (Carlson, Rudgers et al. 2007). Carlson et al. constructed plasmids carrying maize centromeric repeats, delivered purified constructs to embryogenic maize tissue, and assessed their ability to promote the formation of maize minichromosomes (MMCs). MMC1 was characterized in detail; this CentC-based construct contained 19 kb of centromeric DNA and conferred efficient mitotic and meiotic inheritance through at least four generations when introduced into plant cells.

Making artificial chromosomes often requires centromeric sequences specific to a target organism, as sequences from a related organism sometimes do not work efficiently in establishing centromere function (Kitada et al., 1997; Pribylova et al., 2007) Identification of centromeres has been pursued in several organisms by searching for repetitive DNA or methylated DNA followed by labeling studies to determine whether the identified sequences hybridize to the centromere region of chromosomes, and/or functional studies to determine whether the identified sequence(s) function as centromeres (see, for example, U.S. Pat. No. 7,456,013, WO 08/112,972).

### SUMMARY OF THE INVENTION

In a first aspect, the invention is directed to cotton plants, such as, for example, Gossypium hirsutum, Gossypium davidsonii, Gossypium klotzschianum, Gossypium raimondii, Gossypium anomalum, and Gossypium somalense plants, or cotton plants having, for example, B, D, and E2 genome types, comprising a minichromosome, and methods pertaining to such cotton plants, wherein the minichromosome has a transmission efficiency during mitotic or meiotic division of at least 50%, 90%, 95% and 99%, or a transmission efficiency during meiosis of at least 25%, 50%, 70%, 80%, 85%, 90%, 95%, or 99%. The minichromosome can be anywhere from 10 kb to 1000 kb, including 100 and 200 kb. The minichromosome can also comprise a site for site-specific recombination, a centromere nucleic acid insert derived from a cotton plant, where that centromere nucleic acid can be obtained directly from cotton genomic DNA or artificially synthesized. If the minichromosome is derived from a donor clone or centromere clone, it can have substitutions, deletions, insertions, duplications, or arrangements of one or more nucleotides in the minichromosome compared to the nucleotide sequence of the donor clone or centromere clone and retains a minichromosome function. The minichromosome can be obtained by passage of the minichromosome through one or more hosts, such as virus, bacteria, yeast, plant, prokaryotes, or eukaryotes. The minichromosome can further comprise 1, 2, 5, 10 or more exogenous nucleic acids. These exogenous nucleic acids can be operably linked to a heterologous regulatory sequence that functions in plant cells, such as a plant, a non-plant, an insect or a yeast regulatory sequence. Non-plant regulatory sequences can comprise polynucleotides such as SEQ ID NOS:1-20, or a functional fragment thereof. Exogenous nucleic acids carried on minichromosomes can confer resistance to an herbicide (e.g., phosphinothricin or glyphosate), an insect, a disease, or to stress. Examples of such nucleic acids are those that encode phosphinothricin acetyltransferase, glyphosate acetyltransferase or a mutant enoylpyruvyl shikimate phosphate (EPSP) synthase. Other examples include those exogenous nucleic acids that encode a Bacillus thuringiensis or Bacillus cereus toxin gene. An exogenous nucleic acid can also confer resistance to drought, heat, chilling, freezing, excessive moisture, ultraviolet light, ionizing radiation, mechanical stress, toxins, pollution, or salt stress; or, it can confer resistance to a virus, bacteria, fungi or nematode. Since minichromosomes can carry a plurality of exogenous nucleic acids, minichromosome can confer multiple functions simultaneously, such as herbicide and insect resistance.

Examples of exogenous nucleic acids include: a nitrogen fixation gene, a plant stress-induced gene, a nutrient utilization gene, a gene that affects plant pigmentation, a gene that encodes an antisense or ribozyme molecule, a gene encoding a secretable antigen, a toxin gene, a receptor gene, a ligand gene, a seed storage gene, a hormone gene, an enzyme gene, an interleukin gene, a clotting factor gene, a cytokine gene, an antibody gene, a growth factor gene, a transcription factor gene, a transcriptional repressor gene, a DNA-binding protein gene, a recombination gene, a DNA replication gene, a programmed cell death gene, a kinase gene, a phosphatase gene, a G protein gene, a cyclin gene, a cell cycle control gene, a gene involved in transcription, a gene involved in translation, a gene involved in RNA processing, a gene involved in RNAi, an organellar gene, a intracellular trafficking gene, an integral membrane protein gene, a transporter gene, a membrane channel protein gene, a cell wall gene, a gene involved in protein processing, a gene involved in protein modification, a gene involved in protein degradation, a gene involved in metabolism, a gene involved in biosynthesis, a gene involved in assimilation of nitrogen or other elements or nutrients, a gene involved in controlling carbon flux, gene involved in respiration, a gene involved in photosynthesis, a gene involved in light sensing, a gene involved in organogenesis, a gene involved in embryogenesis, a gene involved in differentiation, a gene involved in meiotic drive, a gene involved in self incompatibility, a gene involved in development, a gene involved in nutrient, metabolite or mineral transport, a gene involved in nutrient, metabolite or mineral storage, a calcium-binding protein gene, or a lipid-binding protein gene.

Additional examples of exogenous nucleic acids include those that encode an enzyme involved in or for: metabolizing biochemical wastes for use in bioremediation, modifying pathways that produce secondary plant metabolites, produces a pharmaceutical, improves changes the nutritional content of a plant, vitamin synthesis, carbohydrate, polysaccharide or starch synthesis, mineral accumulation or availability, a gene that encodes a phytase, fatty acid, fat or oil synthesis, synthesis of chemicals or plastics, synthesis of a fuel and synthesis of a fragrance, synthesis of a flavor, synthesis of a pigment or dye, synthesis of a hydrocarbon, synthesis of a structural or fibrous compound, synthesis of a food additive, synthesis of a chemical insecticide, synthesis of an insect repellent, or a gene controlling carbon flux in a plant.

The minichromosome can comprise a centromere comprising n copies of a repeated nucleotide sequence, wherein n is less than 1000, such as 5, 15, and 50. The minichromosome can further comprise a telomere. Minichromosomes can be circular or linear.

The cotton plant parts or cotton plants comprising a minichromosome also comprise an aspect of the invention, such as a root, cutting, stem, stalk, fiber (lint), square, boll, flower, leaf, epidermis, vascular tissue, organ, protoplast, crown, callus culture, petiole, petal, sepal, stamen, stigma, style, bud, meristem, cambium, cortex, pith, sheath, or embryo. In addition, plant parts, such as a meiocyte or gamete or ovule or pollen or endosperm are also included, as are propagules and cells of such cotton plants. The invention is also directed to materials made from cotton from a cotton plant harboring a minichromosome, such as textiles, fibers, yarn, etc.

The invention also includes progeny of such cotton plants comprising a minichromosome, including that progeny that results from self-breeding, cross-breeding, apomyxis, or clonal propagation. These progeny can comprise a minichromosome descended from a parental minichromosome that contained a centromere less than 1, 2, 3, 4, 5, 6, 7, 8, 9, 10, 25, 50, 75, 100, or 150 kb.

In another aspect, the invention is directed to methods of using such cottonplants comprising a minichromosome. These methods include using the cotton plants to produce a food or feed product. Or to produce a recombinant protein that can, for example, also be isolated from such cotton plants. The method can further comprise the steps of harvesting the cotton plant and isolating the recombinant protein from the cotton plant. Examples of recombinant proteins include pharmaceutical proteins. Such cotton plants can also be used in methods to produce a chemical product that can be isolated from the harvested cotton plant. An example of a chemical product is a pharmaceutical product and oils

In another aspect, the invention is directed to isolated polynucleotides comprising a polynucleotide sequence having at least 70%, 79%, 80%, 90%, 95%, and 99% sequence identity with SEQ ID NO:94. Furthermore, this polynucleotide can confer the ability to the polynucleotide or to any polynucleotide to be transmitted through mitosis or meiosis in a plant cell with a transmission efficiency from 1% to greater than 50%. The plant cell can be, for example, a cotton cell. The polynucleotide can consist of a nucleic acid sequence having at least 70% sequence identity with a nucleic acid selected from the group consisting of SEQ ID NOs:95, 96 and 97. Other examples of centromere nucleic acids include those having at least 70%, 79%, 80%, 90%, 95%, and 99% sequence identity, include SEQ ID NOS: 113, 116, 119 and 122, or the sequences themselves.

In another aspect, the invention is directed to isolated polynucleotides comprising a sequence having at least 70% sequence identity to a a nucleic acid sequence selected from the group consisting of SEQ ID NOS:95, 96 and 97. SEQ ID NO:97 can further comprise an insert of 1 bp to 4 kb, such as a sequence of SEQ ID NO:96 or a retrotransposon-like element, and additionally, can confer the ability to the polynucleotide to be transmitted through mitosis or meiosis in a plant cell (such as a cotton plant cell) with a transmission efficiency of at least 50%, 90%, 95% and 99%, or a transmission efficiency during meiosis of at least 25%, 50%, 70%, 80%, 85%, 90%, 95%, or 99%.

In another aspect, the invention is directed to recombinant DNA constructs, such as minichromosomes, comprising polynucleotides comprising a sequence having at least 70% sequence identity to a a nucleic acid sequence selected from the group consisting of SEQ ID NOS:95, 96 and 97. SEQ ID NO:97 can further comprise an insert of 1 bp to 3, 999 bp, such as a sequence of SEQ ID NO:96 or a retrotransposon-like element, and additionally, can confer the ability to the polynucleotide to be transmitted through mitosis or meiosis in a plant cell (such as a cotton plant cell) with a transmission efficiency of at least 50%, 90%, 95% and 99%, or a transmission efficiency during meiosis of at least 25%, 50%, 70%, 80%, 85%, 90%, 95%, or 99%. The invention is further directed to cells, such as plant cells (e.g., a cotton plant cell); and plants, such as crop plants (e.g., cotton) comprising such minichromosomes.

The minichromosome can also comprise a site for site-specific recombination, a centromere nucleic acid insert derived from a cotton plant, where that centromere nucleic acid can be obtained directly from cotton genomic DNA or artificially synthesized. If the minichromosome is derived from a donor clone or centromere clone, it can have substitutions, deletions, insertions, duplications, or arrangements of one or more nucleotides in the minichromosome compared to the nucleotide sequence of the donor clone or centromere clone and retains a minichromosome function. The minichromosome can be obtained by passage of the minichromosome through one or more hosts, such as virus, bacteria, yeast, plant, prokaryotes, or eukaryotes. The minichromosome can further comprise 1, 2, 5, 10 or more exogenous nucleic acids. These exogenous nucleic acids can be operably linked to a he3tyerologous regulatory sequence that functions in plant cells, such as a plant, a non-plant, an insect or a yeast regulatory sequence. Non-plant regulatory sequences can comprise SEQ ID NOS:1-20, or a functional fragment thereof. Exogenouse nucleic acids carried on minichromosomes can confer resistance to a herbicide (e.g., phosphinothricin or glyphosate), an insect, a disease, or to stress. Examples of such nucleic acids are those that encode phosphinothricin acetyltransferase, glyphosate acetyltransferase or a mutant enoylpyruvyl shikimate phosphate (EPSP) synthase. Other examples include those exogenous nucleic acids that encode a Bacillus thuringiensis or Bacillus cereus toxin gene. An exogenous nucleic acid can also confer resistance to drought, heat, chilling, freezing, excessive moisture, ultraviolet light, ionizing radiation, mechanical stress, toxins, pollution, or salt stress; or, it can confer resistance to a virus, bacteria, fungi or nematode. Since minichromosomes can carry a plurality of exogenous nucleic acids, minichromosome can confer multiple functions simultaneously, such as herbicide and insect resistance.

Examples of exogenous nucleic acids include: a nitrogen fixation gene, a plant stress-induced gene, a nutrient utilization gene, a gene that affects plant pigmentation, a gene that encodes an antisense or ribozyme molecule, a gene encoding a secretable antigen, a toxin gene, a receptor gene, a ligand gene, a seed storage gene, a hormone gene, an enzyme gene, an interleukin gene, a clotting factor gene, a cytokine gene, an antibody gene, a growth factor gene, a transcription factor gene, a transcriptional repressor gene, a DNA-binding protein gene, a recombination gene, a DNA replication gene, a programmed cell death gene, a kinase gene, a phosphatase gene, a G protein gene, a cyclin gene, a cell cycle control gene, a gene involved in transcription, a gene involved in translation, a gene involved in RNA processing, a gene involved in RNAi, an organellar gene, a intracellular trafficking gene, an integral membrane protein gene, a transporter gene, a membrane channel protein gene, a cell wall gene, a gene involved in protein processing, a gene involved in protein modification, a gene involved in protein degradation, a gene involved in metabolism, a gene involved in biosynthesis, a gene involved in assimilation of nitrogen or other elements or nutrients, a gene involved in controlling carbon flux, gene involved in respiration, a gene involved in photosynthesis, a gene involved in light sensing, a gene involved in organogenesis, a gene involved in embryogenesis, a gene involved in differentiation, a gene involved in meiotic drive, a gene involved in self incompatibility, a gene involved in development, a gene involved in nutrient, metabolite or mineral transport, a gene involved in nutrient, metabolite or mineral storage, a calcium-binding protein gene, or a lipid-binding protein gene.

Additional examples of exogenous nucleic acids include those that encode an enzyme involved in or for: metabolizing biochemical wastes for use in bioremediation, modifying pathways that produce secondary plant metabolites, produces a pharmaceutical, improves changes the nutritional content of a plant, vitamin synthesis, carbohydrate, polysaccharide or starch synthesis, mineral accumulation or availability, a gene that encodes a phytase, fatty acid, fat or oil synthesis, synthesis of chemicals or plastics, synthesis of a fuel and synthesis of a fragrance, synthesis of a flavor, synthesis of a pigment or dye, synthesis of a hydrocarbon, synthesis of a structural or fibrous compound, synthesis of a food additive, synthesis of a chemical insecticide, synthesis of an insect repellent, or a gene controlling carbon flux in a plant.

In another aspect, the invention is directed to minichromosomes comprising a nucleic acid sequence of SEQ ID NO:94, which can further comprise an insertion of 1 nucleotide to 4 kb. The invention is further directed to cells, such as plant cells (e.g., a cotton plant cell); and plants, such as crop plants (e.g., cotton) comprising such minichromosomes.

In another aspect, the invention is directed to minichromosomes comprising a polynucleotide comprising two copies of a nucleic acid sequence of SEQ ID NO:96, wherein a first copy of SEQ ID NO:96 is located 5′ of SEQ ID NO:95, and the second copy of SEQ ID NO:96 is located 3′ of SEQ ID NO:95. SEQ ID NO:95 can further comprise an insert of 1 nucleotide to 4 kb. The invention is further directed to cells, such as plant cells (e.g., a cotton plant cell); and plants, such as crop plants (e.g., cotton) comprising such minichromosomes.

In another aspect, the invention is directed to minichromosomes comprising a polynucleotide comprising of SEQ ID NO:97. SEQ ID NO:97 can further comprise an insert of 1 nucleotide to 4 kb. The invention is further directed to cells, such as plant cells (e.g., a cotton plant cell); and plants, such as crop plants (e.g., cotton) comprising such minichromosomes.

In yet another aspect, the invention is directed to DNA constructs that comprise a first nucleic acid sequence having at least 70% sequence identity to a nucleic acid sequence selected from the group consisting of SEQ ID NO:94, SEQ ID NO:95 and SEQ ID NO:97. Such DNA constructs can comprise a first nucleic acid sequence having at least 70% sequence identity to SEQ ID NO:95, and further comprises at least two copies of a second nucleic acid sequence having at least 70% sequence identity to SEQ ID NO:96, wherein a first copy of SEQ ID NO:96 is located 5′ of SEQ ID NO:95, and the second copy of SEQ ID NO:96 is located 3′ of SEQ ID NO:95. The first nucleic acid sequence further comprises an insertion of 1 nt to 4 kb. The two copies of SEQ ID NO:96 may be 90% or more identical to each other, or less than 90% identical. The invention also comprises minichromosomes that comprise such DNA constructs, as well as plants comprising such minichromosomes.

In another aspect, the invention is directed to polynucleotides comprising a structure comprising a core sequence flanked by two retroelement-related sequences, wherein the polynucleotide confers an ability to the polynucleotide to be transmitted through mitosis or meiosis in a plant cell with a transmission efficiency greater than 50%. The retroelement-related sequences in such structures can be at least 90% identical or are less than 90% identical. These retroelement-related sequences can have at least 70%, 80%, 90%, 95% sequence identity with SEQ ID NO:96, or consist of SEQ ID NO:96. The core sequence can have at least 70%, 80%, 90%, 95% sequence identity with SEQ ID NO:95. Mitotic or meiotic efficiency can be measured in a plant cells, such as cotton plant cells.

In all minichromosome compositions of the invention, the invention also comprises plant cells, plant parts, plants and products derived from such plants that harbor such minichromosomes. In all aspects of the invention, cotton cells, cotton plants, and centromere sequences can be Gossypium hirsutum, Gossypium davidsonii, Gossypium klotzschianum, Gossypium raimondii, Gossypium anomalum, and Gossypium somalense plants, cotton plant cells, and centromere sequences; or plants, cotton plant cells, or centromere sequences having, for example, B, D, and E2 genome types.

### BRIEF DESCRIPTION OF SEVERAL VIEWS OF THE DRAWINGS

FIGS. 1A-1C show the localization of probes prepared to identify the 194 tandem repeat represented by SEQ ID NO:90 (FIG. 1A), CrGh1 (FIG. 1B), and the overlay of the two signals (FIG. 1C) in mitotic metaphase G. hirsutum chromosomes. The chromosomes are counterstained with DAPI.

FIG. 2 shows the localization of probes in FISH experiments on mitotic metaphase G. hirsutum chromosomes prepare to identify the tandem repeats represented by SEQ ID NO:90/CrGh1 1 (194 bp) and SEQ ID NO:91 2 (210 bp). The chromosomes are counterstained with DAPI.

FIGS. 3A-3B show FISH experiments on mitotic metaphase G. hirsutum chromosomes with a probe that is not specific for cotton centromere.

FIGS. 4A-4I show a diagrammatic representation of a cotton centromere (FIG. 4A), and staining with probes specific for different parts of the cotton centromere in FISH experiments on mitotic metaphase G. hirsutum chromosomes (FIGS. 4B-41).

FIGS. 5A-5I show the structure of the novel cotton centromere and FISH experiments on mitotic metaphase G. hirsutum chromosomes using probes specific to cotton centromeres.

FIGS. 6A-GH show the results of FISH experiments using SEQ ID NO:95- and SEQ ID NO:96-specific probes in G. hirsutum chromosomes.

FIGS. 7A-7D show the results of FISH experiments using a BAC harboring a cotton centromere, BAC 53H10, and a probe specific for SEQ ID NO:96.

### DETAILED DESCRIPTION OF THE INVENTION

### I. Introduction

The invention is based on the surprising result that the centromeres in cotton are structurally distinct from those observed in other plants. Typically plant centromeres are comprised of tandem arrays of repetitive satellite sequences (e.g., the 180 bp repeat in Arabidopsis; CentC in maize; CentO in rice) that are sometimes interspersed with centromere-specific retrotransposon sequences (e.g., CRM in maize and CRR in rice) (Ma and Jackson 2006); Ananiev et al., 1998; Nagaki et al., 2003; Zhong et al., 2002; (Cheng, Dong et al. 2002). Instead cotton centromeres lack long tandem arrays of satellite sequence and arecomprised of retrotransposon-like element structures that include a core sequence flanked by two identical sequences. This surprising observation allows for the production of modified plants, especially cotton plants, containing functional, stable, autonomous MCs. A diagrammatic rendering of the structure of the novel centromeric sequence is shown in FIG. 5A. FIG. 5A shows that the novel cotton centromeric structure has a core (CenCORE; designated as an oval in the drawing) that is flanked by CenFR sequences (designated by arrows). This structure is reminiscient of other centromeric structures found in some in higher, non-plant eukaryotes, such as Drosophila melanogaster. (Sun, Wahlstrom et al. 1997). To date, this structure has never been observed in any plant to function as a centromere.

### II. Definitions

“Adchromosomal” plant or plant part means a plant or plant part that contains functional, stable and autonomous MCs. Adchromosomal plants or plant parts can be chimeric or not chimeric (chimeric meaning that MCs are only in certain portions of the plant, and are not uniformly distributed throughout the plant). An adchromosomal plant cell contains at least one functional, stable and autonomous MC.

“Autonomous” means that when delivered to plant cells, at least some MCs are transmitted through mitotic division to daughter cells and are episomal in the daughter plant cells, i.e., are not chromosomally integrated in the daughter plant cells. Daughter plant cells that contain autonomous MCs can be selected for further propagation using, for example, selectable or screenable markers. During the introduction into a cell of a MC, or during subsequent stages of the cell cycle, there may be chromosomal integration of some portion or all of the DNA derived from a MC in some cells. The MC is still characterized as autonomous despite the occurrence of such events if a plant, plant part or plant tissue can be regenerated that contains episomal descendants of the MC distributed throughout its parts, or if gametes or progeny can be derived from the plant that contain episomal descendants of the MC distributed through its parts.

“Centromere” is any DNA sequence that confers an ability to segregate to daughter cells through cell division. This sequence can produce a transmission efficiency to daughter cells ranging from about 1% to about 100%, including to about 5%, 10%, 20%, 30%, 40%, 50%, 60%, 70%, 80%, 90% or about 95% of daughter cells. Variations in transmission efficiency can find important applications within the scope of the invention; for example, MCs carrying centromeres that confer 100% stability could be maintained in all daughter cells without selection, while those that confer 1% stability could be temporarily introduced into a transgenic organism, but later eliminated when desired. In particular embodiments of the invention, the centromere can confer stable transmission to daughter cells of a nucleic acid sequence, including a recombinant construct comprising the centromere, through mitotic or meiotic divisions, including through both mitotic and meiotic divisions. A plant centromere is not necessarily derived from plants, but has the ability to promote DNA transmission to daughter plant cells.

“Circular permutations” refer to variants of a sequence that begin at base n within the sequence, proceed to the end of the sequence, resume with base number one of the sequence, and proceed to base n−1. For this analysis, n can be any number less than or equal to the length of the sequence. For example, circular permutations of the sequence ABCD are: ABCD, BCDA, CDAB, and DABC.

“Co-delivery” refers to the delivery of two nucleic acid segments to a cell. The segments can be delivered simultaneously or sequentially. The segments can be the same kind of vector (e.g. two MCs) or different (e.g. a combination of MC, T-DNA, viral vector, plasmid vector, etc.). Alternatively, the segments can be co-delivered on a single vector.

“Consensus” refers to a nucleic acid sequence derived by comparing two or more related sequences. A consensus sequence defines both the conserved and variable sites between the sequences being compared. Any one of the sequences used to derive the consensus or any permutation defined by the consensus can be useful in construction of MCs.

“Exogenous” when used in reference to a nucleic acid, for example, refers to any nucleic acid that has been introduced into a recipient cell, regardless of whether the same or similar nucleic acid is already present in such a cell. An “exogenous gene” can be a gene not normally found in the host genome in an identical context, or an extra copy of a host gene. The gene can be isolated from a different species than that of the host genome, or alternatively, isolated from the host genome but operably linked to one or more regulatory regions that differ from those found in the unaltered, native gene. The gene can also be synthesized in vitro.

“Functional” when referring to a MC, centromere, nucleic acid, or polypeptide, for example, retains a biological and/or an immunological activity of native or naturally-occurring chromosome, centromere, nucleic acid, or polypeptide, respectively. When used to describe an exogenous nucleic acid carried on an MC, “functional” means that the exogenous nucleic acid can function in a detectable manner when the MC is within a cell, such as a plant cell; exemplary functions of the exogenous nucleic acid include transcription of the exogenous nucleic acid, expression of the exogenous nucleic acid, regulatory control of expression of other exogenous nucleic acids, recognition by a restriction enzyme or other endonuclease, ribozyme or recombinase; providing a substrate for DNA methylation, DNA glycolation or other DNA chemical modification; binding to proteins such as histones, helix-loop-helix proteins, zinc binding proteins, leucine zipper proteins, MADS box proteins, topoisomerases, helicases, transposases, TATA box binding proteins, viral protein, reverse transcriptases, or cohesins; providing an integration site for homologous recombination; providing an integration site for a transposon, T-DNA or retrovirus; providing a substrate for RNAi synthesis; priming of DNA replication; aptamer binding; or kinetochore binding. If multiple exogenous nucleic acids are present within the MC, the function of one or preferably more of the exogenous nucleic acids can be detected under suitable conditions permitting function.

“Linker” refers to a DNA molecule, generally up to 50 or 60 nucleotides long, although linkers can be much larger, such as 100 bp, 1 kb, 100 kb, 1 Gb, etc., and composed of two or more complementary oligonucleotides that have been synthesized chemically, or excised or amplified from existing plasmids or vectors. In a preferred embodiment, this fragment contains one, or preferably more than one, restriction enzyme site for a blunt cutting enzyme and/or a staggered cutting enzyme, such as BamHI. One end of the linker is designed to be ligatable to one end of a linear DNA molecule and the other end is designed to be ligatable to the other end of the linear molecule, or both ends can be designed to be iigatable lo both ends of the linear DNA molecule.

A “mini-chromosome” (“MC”) is a recombinant DNA construct including a centromere and capable of transmission to daughter cells. A MC can remain separate from the host genome (as episomes) or can integrate into host chromosomes. The stability of this construct through cell division could range between from about 1% to about 100%, including about 5%, 10%, 20%, 30%, 40%, 50%, 60%, 70%, 80%, 90% and about 95%. The MC construct can be a circular or linear molecule. It can include elements such as one or more telomeres, origin of replication sequences, stuffer sequences, buffer sequences, chromatin packaging sequences, linkers and genes. The number of such sequences included is only limited by the physical size limitations of the construct itself. It can contain DNA derived from a natural centromere, although it can be preferable to limit the amount of DNA to the minimal amount required to obtain a transmission efficiency in the range of 1-100%. The MC can also contain a synthetic centromere composed of tandem arrays of repeats of any sequence, either derived from a natural centromere, or of synthetic DNA. The MC can also contain DNA derived from multiple natural centromeres. The MC can be inherited through mitosis or meiosis, or through both meiosis and mitosis. The term MC specifically encompasses and includes the terms “plant artificial chromosome” or “PLAC,” or engineered chromosomes or microchromosomes and all teachings relevant to a PLAC or plant artificial chromosome specifically apply to constructs within the meaning of the term MC.

“Non-protein expressing sequence” or “non-protein coding sequence” is defined herein as a nucleic acid sequence that is not eventually translated into protein. The nucleic acid can or can not be transcribed into RNA. Exemplary sequences include ribozymes or antisense RNA.

“Operably linked” is defined herein as a configuration in that a control sequence, e.g., a promoter sequence, directs transcription or translation of another sequence, for example a coding sequence. For example, a promoter sequence could be appropriately placed at a position relative to a coding sequence such that the control sequence directs the production of a polypeptide encoded by the coding sequence.

The term “plant,” as used herein, refers to any type of plant. Exemplary types of plants are listed below, but other types of plants will be known to those of skill in the art and could be used with the invention. Modified plants of the invention include, for example, dicots, gymnosperm, monocots, mosses, ferns, horsetails, club mosses, liver worts, homworts, red algae, brown algae, gametophytes and sporophytes of pteridophytes, and green algae.

A common class of plants exploited in agriculture are vegetable crops, including artichokes, kohlrabi, arugula, leeks, asparagus, lettuce (e.g., head, leaf, romaine), bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, Chinese cabbage, peppers, collards, potatoes, cucumber plants (marrows, cucumbers), pumpkins, cucurbits, radishes, dry bulb onions, rutabaga, eggplant, salsify, escarole, shallots, endive, garlic, spinach, green onions, squash, greens, beet (sugar beet or fodder beet), sweet potatoes, swiss chard, horseradish, tomatoes, kale, turnips, or spices.

Other types of plants frequently finding commercial use include fruit and vine crops such as apples, grapes, apricots, cherries, nectarines, peaches, pears, plums, prunes, quince, almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, or lychee.

Modified wood and fiber or pulp plants of particular interest include, but are not limited to maple, oak, cherry, mahogany, poplar, aspen, birch, beech, spruce, fir, kenaf, pine, walnut, cedar, redwood, chestnut, acacia, bombax, alder, eucalyptus, catalpa, mulberry, persimmon, ash, honeylocust, sweetgum, privet, sycamore, magnolia, sourwood, cottonwood, mesquite, buckthorn, locust, willow, elderberry, teak, linden, bubinga, basswood or elm.

Modified flowers and ornamental plants of particular interest, include roses, petunias, pansy, peony, olive, begonias, violets, phlox, nasturtiums, irises, lilies, orchids, vinca, philodendron, poinscttias, opuntia, cyclamen, magnolia, dogwood, azalea, redbud, boxwood, Viburnum, maple, elderberry, hosta, agave, asters, sunflower, pansies, hibiscus, morning glory, alstromeria, zinnia, geranium, Prosopis, artemesia, clematis, delphinium, dianthus, gallium, coreopsis, iberis, lamium, poppy, lavender, leucophyllum, scdum, salvia, verbascum, digitalis, penstemon, savory, pythrethrum, or oenolhera. Modified nut-bearing trees of particular interest include, but are not limited to pecans, walnuts, macadamia nuts, hazelnuts, almonds, or pistachios, cashews, pignolas or chestnuts.

Many of the most widely grown plants are field crop plants such as evening primrose, meadow foam, corn (field, sweet, popcorn), hops, jojoba, peanuts, rice, safflower, small grains (barley, oats, rye, wheat, etc.), sorghum, tobacco, kapok, leguminous plants (beans, lentils, peas, soybeans), oil plants (rape, mustard, poppy, olives, sunflowers, coconut, castor oil plants, cocoa beans, groundnuts, oil palms), fibre plants (cotton, flax, hemp, jute), lauraceae (cinnamon, camphor), or plants such as coffee, sugarcane, cocoa, tea, or natural rubber plants.

Still other examples of plants include bedding plants such as flowers, cactus, succulents or ornamental plants, as well as trees such as forest (broad-leaved trees or evergreens, such as conifers), fruit, ornamental, or nut-bearing trees, as well as shrubs or other nursery stock.

Modified crop plants of particular interest in the present invention include soybean (Glycine max), cotton, canola (also known as rape), wheat, sunflower, sorghum, alfalfa, barley, safflower, millet, rice, tobacco, fruit and vegetable crops or turfgrasses. Exemplary cereals include maize, wheat, barley, oats, rye, millet, sorghum, rice triticale, secale, einkorn, spelt, emmer, teff, milo, flax, gramma grass, Tripsacum sp., or teosinte. Oil-producing plants include plant species that produce and store triacylglycerol in specific organs, primarily in seeds. Such species include soybean (Glycine max), rapeseed or canola (including Brassica napus, Brassica rapa or Brassica campestris), Brassica juncea, Brassica carinata, sunflower (Helianthus annuus), cotton (including Gossypium hirsutum), corn (Zea mays), cocoa (Theobroma cacao), safflower (Carthamus tinctorius), oil palm (Elaeis guineensis), coconut palm (Cocos nucifera), flax (Linum usitatissimum), castor (Ricinus communis) or peanut (Arachis hypogaea).

“Cotton” includes species of the genus Gossypium, including the commercially important cottons, Gossypium hirsutum (Upland cotton), Gossypium herbaceum (Levant cotton), Gossypium arboreum (Tree cotton), and Gossypium barbadense (Pima cotton), as well as the non-commercial species, including Gossypium darwinii (Darwin's cotton), Gossypium tomentosum (Hawaiian cotton), Gossypium australe (Australian cotton), Gossypium sturtianum (Sturt's Desert Rose), Gossypium thurberi (Arizona wild cotton), Gossypium anomalum, Gossypium triphyllum, Gossypium somalense, Gossypium stocksii, Gossypium longicalyx, Gossypium robinsonii, Gossypium bickii, Gossypium capitis-viridis, Gossypium trifurcatum, Gossypium armourianum, Gossypium harknessii, Gossypium davidsonii, Gossypium klotschianum, Gossypium aridum, Gossypium gossypioides, Gossypium lobatum, Gossypium trilobatum, Gossypium laxum, Gossypium turneri, Gossypium schwendimanii, Gossypium areysianum, Gossypium incanum, Gossypium trifurcatum, Gossypium benidirense, Gossypium bricchettii, Gossypium vollesenii, Gossypium trifurcatum, Gossypium nelsonii, Gossypium anapoides, Gossypium costulatum, Gossypium cunninghamii, Gossypium enthyle, Gossypium exiguum, Gossypium londonderriense, Gossypium marchantii, Gossypium nobile, Gossypium pilosum, Gossypium populifolium, Gossypium pulchellum, Gossypium rotundifolium, Gossypium mustelinum and Gossypium raimondii. Most cotton is diploid; however, several species are tetraploid, including the commercially important G. hirsutum and G. barbadense. Other tetraploid species include G. tomentosum, G. mustelinum, and G. darwinii. Cotton, as used herein, also includes any hybrid between these species, and any polyploidy derived from these species. Cotton can also be subdived into genome types or groups. Diploid species (2n=26) fall into eight genomic groups (A-G, and K). The African Glade, comprising the A, B, E, and F genomes, occurs naturally in Africa and Asia, while the D genome Glade is indigenous to the Americas. A third diploid Glade, including C, G, and K, is found in Australia. Polyploid cotton, such as G. hirsutum, can contain more than one genomic group (such as A and D).

“Plant part” includes pollen, silk, endosperm, ovule, seed, embryo, pods, roots, cuttings, tubers, stems, stalks, fiber (lint), square, boll, fruit, berries, nuts, flowers, leaves, bark, wood, whole plant, plant cell, plant organ, epidermis, vascular tissue, protoplast, cell culture, crown, callus culture, petiole, petal, sepal, stamen, stigma, style, bud, meristem, cambium, cortex, pith, sheath, or any group of plant cells organized into a structural and functional unit. In one preferred embodiment, the exogenous nucleic acid is expressed in a specific location or tissue of a plant, for example, epidermis, vascular tissue, meristem, cambium, cortex, pith, leaf, sheath, flower, root or seed.

“Promoter” is a DNA sequence that allows the binding of RNA polymerase (including but not limited to RNA polymerase I, RNA polymerase II and RNA polymerase III from eukaryotes), and optionally other accessory or regulatory factors, and directs the polymerase to a downstream transcriptional start site of a nucleic acid sequence encoding a polypeptide to initiate transcription. RNA polymerase effectively catalyzes the assembly of messenger RNA complementary to the appropriate DNA strand of the coding region.

A “promoter operably linked to a heterologous gene” is a promoter that is operably linked to a gene that is different from the gene to that the promoter is normally operably linked in its native state. Similarly, an “exogenous nucleic acid operably linked to a heterologous regulatory sequence” is a nucleic acid that is operably linked to a regulatory control sequence to that it is not normally linked in its native state.

“Hybrid promoter” means parts of two or more promoters that are fused together to generate a sequence that is a fusion of the two or more promoters, that is operably linked to a coding sequence and mediates the transcription of the coding sequence into mRNA.

“Tandem promoter” means two or more promoter sequences each of that is operably linked to a coding sequence and mediates the transcription of the coding sequence into mRNA.

“Constitutive active promoter” means a promoter that allows permanent and stable expression of the gene of interest.

“Inducible promoter” means a promoter induced by the presence or absence of a biotic or an abiotic factor.

“Polypeptide” does not refer to a specific length of the encoded product and, therefore, encompasses peptides, oligopeptides, and proteins. “Exogenous polypeptide” means a polypeptide that is not native to the plant cell, a native polypeptide in that modifications have been made to alter the native sequence, or a native polypeptide whose expression is quantitatively altered as a result of a manipulation of the plant cell by recombinant DNA techniques.

“Pseudogene” refers to a non-functional copy of a protein-coding gene; pseudogenes found in the genomes of eukaryotic organisms are often inactivated by mutations and are thus presumed to be non-essential to that organism; pseudogenes of reverse transcriptase and other open reading frames found in retroelements are abundant in the centromeric regions of Arabidopsis and other organisms and are often present in complex clusters of related sequences.

“Regulatory sequence” refers to any DNA sequence that influences the efficiency of transcription or translation of any gene. The term includes sequences comprising promoters, enhancers and terminators.

“Repeated nucleotide sequence” refers to any nucleic acid sequence of at least 25 bp present in a genome or a recombinant molecule, other than a telomere repeat, that occurs at least two or more times and that are preferably at least 80% identical either in head to tail or head to head orientation either with or without intervening sequence between repeat units.

“Retroelement” or “retrotransposon” refers to a genetic element related to retroviruses that disperse through an RNA stage; the abundant retroelements present in plant genomes contain long terminal repeats (LTR retrotransposons) and encode a polyprotein gene that is processed into several proteins including a reverse transcriptase. Specific retroelements (complete or partial sequences (e.g., “retroelement-like sequence,” “retrotransposon-like sequence,” and “retroelement-derived sequence”) can be found in and around plant centromeres and can be present as dispersed copies or complex repeat clusters. Individual copies of retroelements can be truncated or contain mutations; intact retrolements are rarely encountered.

“Satellite DNA” refers to short DNA sequences (typically <1000 bp) present in a genome as multiple repeats, mostly arranged in a tandemly repeated fashion, as opposed to a dispersed fashion. Repetitive arrays of specific satellite repeats are abundant in the centromeres of many higher eukaryotic organisms.

“Screenable marker” is a gene whose presence results in an identifiable phenotype. This phenotype can be observed under standard conditions, altered conditions such as elevated temperature, or in the presence of certain chemicals used to detect the phenotype. The use of a screenable marker allows for the use of lower, sub-killing antibiotic concentrations and the use of a visible marker gene to identify clusters of transformed cells, and then manipulation of these cells to homogeneity. Examples of screenable markers include genes that encode fluorescent proteins that are detectable by a visual microscope such as the fluorescent reporter genes DsRed, ZsGreen, ZsYellow, AmCyan, Green Fluorescent Protein (GFP). An additional preferred screenable marker gene is lac.

The invention also contemplates novel methods of screening for adchromosomal plant cells that involve use of relatively low, sub-killing concentrations of a selection agent (e.g., sub-killing antibiotic concentrations), and also involve use of a screenable marker (e.g., a visible marker gene) to identify clusters of modified cells carrying the screenable marker, after that these screenable cells are manipulated to homogeneity. A “selectable marker” is a gene whose presence results in a clear phenotype, and most often a growth advantage for cells that contain the marker. This growth advantage can be present under standard conditions, altered conditions such as elevated temperature, specialized media compositions, or in the presence of certain chemicals such as herbicides or antibiotics. Examples of selectable markers include the thymidine kinase gene, the cellular adenine phosphoribosyltransferase gene and the dihydrylfblatc rcductase gene, hygromycin phosphotransferase genes, bar, neomycin phosphotransferase genes and phosphomannose isomerase, among others. Especially useful selectable markers in the present invention include genes whose expression confer antibiotic or herbicide resistance to the host cell, or proteins allowing utilization of a carbon source not normally utilized by plant cells. Especially useful are proteins conferring cellular resistance to kanamycin, G 418, paramomycin, hygromycin, bialaphos, and glyphosate for example, or proteins allowing utilization of a carbon source, such as mannose, not normally utilized by plant cells.

“Stable” means that a MC can be transmitted to daughter cells over at least 8 mitotic generations. Some embodiments of MCs can be transmitted as functional, autonomous units for less than 8 mitotic generations, e.g., 1, 2, 3, 4, 5, 6, or 7. Preferred MCs can be transmitted over at least 8, 9, 10, 11, 12, 13, 14, 15, 16, 17, 18, 18, 19, 20, 21, 22, 23, 24, 25, 26, 27, 28, 29 or 30 mitotic generations, for example, through the regeneration or differentiation of an entire plant, and preferably are transmitted through meiotic division to gametes. Other preferred MCs can be further maintained in the zygote derived from such a gamete or in an embryo or endosperm derived from one or more such gametes. A “functional and stable” MC is one in that functional MCs can be detected after transmission of the MCs over at least 8 mitotic generations, or after inheritance through a meiotic division. During mitotic division, as occurs occasionally with native chromosomes, there can be some non-transmission of MCs; the MC can still be characterized as stable despite the occurrence of such events if an adchromosomal plant that contains descendants of the MC distributed throughout its parts can be regenerated from cells, cuttings, propagules, or cell cultures containing the MC, or if an adchromosomal plant can be identified in progeny of the plant containing the MC.

“Structural gene” is a sequence that codes for a polypeptide or RNA and includes 5′ and 3′ ends. The structural gene can be from the host into which the structural gene is transformed or from another species. A structural gene usually includes one or more regulatory sequences that modulate the expression of the structural gene, such as a promoter, terminator or enhancer. Structural genes often confer some useful phenotype upon an organism comprising the structural gene, for example, herbicide resistance. A structural gene can encode an RNA sequence that is not translated into a protein, for example a tRNA or rRNA gene.

“Synthetic,” when used in the context of a polynucleotide or polypeptide, refers to a molecule that is made using standard synthetic techniques, e.g., using an automated DNA or peptide synthesizer. Synthetic sequence can be a native sequence, or a modified sequence.

“Telomere” or “telomere DNA” refers to a sequence capable of capping the ends of a chromosome, thereby preventing degradation of the chromosome end, ensuring replication and preventing fusion to other chromosome sequences. Telomeres can include naturally occurring telomere sequences or synthetic sequences. Telomeres from one species can confer telomere activity in another species. An exemplary telomere DNA is a heptanucleotide telomere repeat TTTAGGG (SEQ ID NO:98; and its complement) found in the majority of plants.

“Trait” refers either to the altered phenotype of interest or the nucleic acid that causes the altered phenotype of interest.

“Transformed,” “transgenic,” “modified,” and “recombinant” refer to a host organism such as a plant into which an exogenous or heterologous nucleic acid molecule has been introduced, and includes whole plants, meiocytes, seeds, zygotes, embryos, endosperm, or progeny of such plants that retain the exogenous or heterologous nucleic acid molecule but that have not themselves been subjected to the transformation process.

When the phrase “transmission efficiency” of a certain percent is used, transmission percent efficiency is calculated by measuring MC presence through one or more mitotic or meiotic generations. It is directly measured as the ratio (expressed as a percentage) of the daughter cells or plants demonstrating presence of the MC to parental cells or plants demonstrating presence of the MC. Presence of the MC in parental and daughter cells is demonstrated with assays that detect the presence of an exogenous nucleic acid carried on the MC. Exemplary assays can be the detection of a scrcenable marker (e.g., presence of a fluorescent protein or any gene whose expression results in an observable phenotype), a selectable marker, or PCR amplification of any exogenous nucleic acid carried on the MC.

### III. Making and Using the Invention

### A. Selected Embodiments

The following embodiments are not meant to limit the invention in any way.

One aspect of the invention is related to plants containing functional, stable, autonomous MCs, preferably carrying one or more exogenous nucleic acids. Such plants carrying MCs are contrasted to transgenic plants with genomes that have been altered by chromosomal integration of an exogenous nucleic acid. Expression of the exogenous nucleic acid results in an altered phenotype of the plant. The invention provides for MCs comprising at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 110, 120, 130, 140, 150, 250, 500, 1000 or more exogenous nucleic acids.

Any plants, including bryophytes, algae, seedless vascular plants, monocots, dicots, gymnosperm, field crops, vegetable crops, fruit and vine crops, can be modified by carrying autonomous MCs. Plant parts or plant tissues, including pollen, silk, endosperm, ovule, seed, embryo, pods, roots, cuttings, tubers, stems, stalks, fiber (lint), square, boll, fruit, berries, nuts, flowers, leaves, bark, epidermis, vascular tissue, whole plant, plant cell, plant organ, protoplast, crown, callus culture, petiole, petal, sepal, stamen, stigma, style, bud, meristem, cambium, cortex, pith, sheath, cell culture, or any group of plant cells organized into a structural and functional unit, any cells of can carry MCs.

A related aspect of the invention is adchromosomal plant parts or plant tissues, including pollen, silk, endosperm, ovule, seed, embryo, pods, roots, cuttings, tubers, stems, stalks, crown, fiber (lint), square, boll, callus culture, petiole, petal, sepal, stamen, stigma, style, bud, fruit, berries, nuts, flowers, leaves, bark, wood, whole plant, plant cell, plant organ, protoplast, cell culture, or any group of plant cells organized into a structural and functional unit. In one preferred embodiment, the exogenous nucleic acid is primarily expressed in a specific location or tissue of a plant, for example, epidermis, fiber (lint), boll, square, vascular tissue, meristem, cambium, cortex, pith, leaf, sheath, flower, root or seed. Tissue-specific expression can be accomplished with, for example, localized presence of the MC, selective maintenance of the MC, or with promoters that drive tissue-specific expression.

Another related aspect of the invention is meiocytes, pollen, ovules, endosperm, seed, somatic embryos, apomyctic embryos, embryos derived from fertilization, vegetative propagules and progeny of the originally adchromosomal plant and of its filial generations that retain the functional, stable, autonomous MC. Such progeny include clonally propagated plants, embryos and plant parts as well as filial progeny from self- and cross-breeding, and from apomyxis.

The MC can be transmitted to subsequent generations of viable daughter cells during mitotic cell division with a transmission efficiency of at least 1%, 5%, 10%, 20%, 30%, 40%, 50%, 60%, 70%, 80%, 85%, 90%, 95%, 96%, 97%, 98%, or 99%. The MC is transmitted to viable gametes during meiotic cell division with a transmission efficiency of at least 1%, 5%, 10%, 20%, 30%, 40%, 50%, 60%, 70%, 80%, 85%, 90%, 95% o, 96%, 97%, 98%, or 99% when more than one copy of the MC is present in the gamete mother cells of the plant. The MC is transmitted to viable gametes during meiotic cell division with a transmission frequency of at least 1%, 5%, 10%, 20%, 30%, 40%, 45%, 46%, 47%, 48%, or 49% when one copy of the MC is present in the gamete mother cells of the plant and meiosis produces four viable products (e.g. typical male meiosis) When meiosis produces fewer than four viable products (e.g. typical female meiosis) a phenomenon called meiotic drive can cause the preferential segregation of particular chromosomes into the viable product resulting in higher than expected transmission frequencies of monoosmes through meiosis including at least 51%, 60%, 70%, 80%, 90% 95%, 96%, 97%, 98%, or 99%. For production of seeds via sexual reproduction or by apomyxis, the MC can be transferred into at least 1%, 5%, 10%, 20%, 25%, 30%, 40%, 50%, 60%, 70%, 80%, 85%, 90%, 95%, 96%, 97%, 98%, or 99% of viable embryos when cells of the plant contain more than one copy of the MC. For sexual seed production or apomyxitic seed production from plants with one MC per cell, the MC can be transferred into at least 1%, 5%, 10%, 20%, 30%, 40%, 50%, 60%, 70%, 71%, 72%, 73%, 74%, 75% of viable embryos.

A MC that comprises an exogenous selectable trait or exogenous selectable marker can be used to increase the frequency in subsequent generations of adchromosomal cells, tissues, gametes, embryos, endosperm, seeds, plants or progeny. For example, the frequency of transmission of MCs into viable cells, tissues, gametes, embryos, endosperm, seeds, plants or progeny can be significantly increased after mitosis or meiosis by applying a selection that favors the survival of adchromosomal cells, tissues, gametes, embryos, endosperm, seeds, plants or progeny over cells, tissues, gametes, embryos, endosperm, seeds, plants or progeny lacking the MC.

Transmission efficiency can be measured as the percentage of progeny cells or plants that carry the MC by one of several assays, including detecting expression of a reporter gene (e.g., a gene encoding a fluorescenct protein), PCR detection of a sequence that is carried by the MC, RT-PCR detection of a gene transcript for a gene carried on the MC, Western analysis of a protein produced by a gene carried on the MC, Southern analysis of the DNA (either in total or a portion thereof) carried by the MC, fluorescence in situ hybridization (FISH) or in situ localization by repressor binding. Efficient transmission as measured by some benchmark percentage indicates the degree to which the MC is stable through the mitotic and meiotic cycles. Plants of the invention can also contain chromosomally integrated exogenous nucleic acid in addition to the autonomous MCs. The adchromosomal plants or plant parts, including plant tissues, can include plants that have chromosomal integration of some portion of the MC (e.g., exogenous nucleic acid or centromere sequence) in some or all cells of the plant. The plant, including plant tissue or plant cell, is still characterized as adchromosomal, despite the occurrence of some chromosomal integration. An adchromosomal plant can also have a MC plus non-MC integrated DNA. For example, a standard integrated transgenic plant that subsequently has a MC delivered to it (by crossing or transformation) is an adchromosomal plant. Similarly, an adchromosomal plant that has an integrative transgene delivered to one or more of its chromosomes (including plastid or organellar chromosomes) remains an adchromosomal plant by virtue of the presence of the autonomous MC. In one aspect, the autonomous MC can be isolated from integrated exogenous nucleic acid by crossing the adchromosomal plant containing the integrated exogenous nucleic acid with plants producing some gametes lacking the integrated exogenous nucleic acid and subsequently isolating offspring of the cross, or subsequent crosses, that are adchromosomal but lack the integrated exogenous nucleic acid. This independent segregation of the MC is one measure of the autonomous nature of the MC.

Another aspect of the invention relates to methods for producing and isolating such adchromosomal plants containing functional, stable, autonomous MCs.

In one embodiment, the invention contemplates improved methods for isolating native centromere sequences. In another embodiment, the invention contemplates methods for generating variants of native or artificial centromere sequences by passage through bacterial or plant or other host cells.

In yet another embodiment, the invention contemplates improved methods for regenerating plants, including methods for co-delivery of growth-inducing genes with MCs. The growth delivery genes include Agrobacterium tumefaciens or Arhizogenes isopentenyl transferase (IPT) genes involved in cytokinin biosynthesis, plant IPT genes involved in cytokinin biosynthesis (from any plant), Agrobacterium tumefaciens IAAH, IAAM genes involved in auxin biosynthesis (indole-3-acetamide hydrolase and tryptophan-2-monooxygenase, respectively), Agrobacterium rhizogenes rolA, rolB and rolC genes involved in root formation, Agrobacterium tumefaciens Aux1, Aux2 genes involved in auxin biosynthesis (indole-3-acetamide hydrolase or tryptophan-2-monooxygenase genes), Arabidopsis thaliana leafy cotyledon genes (e.g., Lec1, Lec2) promoting embryogenesis and shoot formation, Arabidopsis thaliana ESR1 gene involved in shoot formation (Banno et al., 2001), Arabidopsis thaliana PGA6/WUSCHEL gene involved in embryogenesis (Zuo, Niu et al. 2002).

The invention further provides isolated promoter nucleic acid sequences comprising any one of SEQ ID NOS:1 to 20, or fragments or variants thereof that retain expression-promoting activity. MCs comprising non-plant promoter sequences such as these that are operably linked to plant-expressed genes (e.g., genes that confer a different phenotype on plants), are contemplated as are plants comprising such MCs.

Another aspect of the invention relates to methods for using such adchromosomal plants containing a MC for producing animal feed, food products, pharmaceutical products and chemical products by appropriate expression of exogenous nucleic acid(s) contained within a MC.

In some animal systems it has been possible to use MCs with centromeres from one species in the cells of a different species (Cavaliere, Scoarughi et al. 2009). Thus, another aspect of the invention is an adchromosomal plant comprising a functional, stable, autonomous MC that contains centromere sequence derived from a different taxonomic plant species, or derived from a different taxonomic plant species, genus, family, order or class.

Yet another aspect of the invention provides novel autonomous MCs used to transform plant cells that are in turn used to generate a plant (or multiple plants). Exemplary MCs of the invention are contemplated to be of a size 2000 kb or less. Other exemplary sizes of MCs include less than or equal to, e.g., 1500 kb, 1000 kb, 900 kb, 800 kb, 700 kb, 600 kb, 500 kb, 450 kb, 400 kb, 350 kb, 300 kb, 250 kb, 200 kb, 150 kb, 100 kb, 90 kb, 80 kb, 70 kb, 60 kb, or 40 kb.

Novel centromere compositions as characterized by sequence content, size, spatial arrangement of sequence motifs, or other parameters. Preferably, the minimal size of centromeric sequence is used in MC construction. Exemplary sizes include a centromeric nucleic acid insert derived from a portion of plant genomic DNA, that is less than or equal to 1000 kb, 900 kb, 800 kb, 700 kb, 600 kb, 500 kb, 400 kb, 300 kb, 200 kb, 150 kb, 100 kb, 95 kb, 90 kb, 85 kb, 80 kb, 75 kb, 70 kb, 65 kb, 60 kb, 55 kb, 50 kb, 45 kb, 40 kb, 35 kb, 30 kb, 25 kb, 20 kb, 15 kb, 10 kb, 5 kb, 4 kb, 3 kb, 2 kb, or 1 kb.

The invention also contemplates MCs or other vectors comprising fragments or variants of the genomic DNA inserts of the described BAC clones, or naturally occurring descendants thereof, that retain the ability to segregate during mitotic or meiotic division, as well as adchromosomal plants or parts containing these MCs. Other exemplary embodiments include fragments or variants of the genomic DNA inserts of any of the identified BAC clones, or descendants thereof, and fragments or variants of the centromeric nucleic acid inserts of any of the vectors or MCs identified herein.

In other exemplary embodiments, the invention contemplates MCs or other vectors comprising centromeric nucleotide sequence that when hybridized to 1, 2, 3, 4, 5, 6, 7, 8, 9, 10, 11, 12, 13, 14, 15, 16, 17, 18, 19, 20, 30, 40, 50, 60, 70, 80, 90, 100 or more probes, including thosedescribed in the Examples, under hybridization conditions described herein, e.g., low, medium or high stringency, provides relative hybridization scores as described in the Examples.

### B. Composition of MCs and MC Construction

The MC vector of the present invention can contain a variety of elements, including: (1) sequences that function as plant centromeres; (2) one or more exogenous nucleic acids; (3) sequences that function as an origin of replication, that can be included in the region that functions as plant centromere, and optional; (4) a bacterial plasmid backbone for propagation of the plasmid in bacteria, though this element may be designed to be removed prior to delivery to a plant cell; (5) sequences that function as plant telomeres (particularly if the MC is linear); (6) optionally, additional “stuffer DNA” sequences that serve to separate the various components on the MC from each other; (7) optionally, “buffer” sequences such as MARs or SARs; (8) optionally, marker sequences of any origin, including but not limited to plant and bacterial origin; (9) optionally, sequences that serve as recombination sites; and (10) optionally, “chromatin packaging sequences” such as cohesion and condensing binding sites.

### C. Novel Centromere Compositions

The centromere in the MC of the present invention can comprise novel repeating centromeric sequences, such as those of SEQ ID NOS:90-92. Alternatively, the centromere of the MCs of the present invention comprise the nucleic acid sequence of SEQ ID NO:94, or portion of SEQ ID NO:94 that confers centromere function. Alternatively, the centromere of the MCs of the present invention comprise thenucleic acid sequence of SEQ ID NO:97. Alternatively, the centromere of the MCs of the present invention comprise the CenCORE sequence (SEQ ID NO:95) in isolation or flanked by CenFL (SEQ ID NO:96) sequence at both the 5′ and 3′ ends, either directly adjacent to SEQ ID NO:95, or separated by intervening sequence. In yet other embodiments, the centromere of the MCs of the present invention comprises SEQ ID NO:97 or SEQ ID NO:95, wherein these sequences further comprise an insertion of 1 bp to 3 kb, wherein the insertion is of any nucleic acid sequence. Alternatively, the centromere of the MCs of the present invention can comprise multiple copies of SEQ ID NO: 94, or SEQ ID NO:95, or SEQ ID NO:96, or SEQ ID NO:97 arrayed tandemly in a head-to-head or head-to-tail orientation with or without interviening nucleic acid sequences. Alternatively, the centromere of the MCs of the present invention can comprise a combination of two or more of SEQ ID NO: 94, or SEQ ID NO:95, or SEQ ID NO:96, or SEQ ID NO:97. All possible combinations of these sequence elements are envisioned, including combinations in which any individual element type is represented more than once.

Exemplary embodiments of centromere nucleic acid sequences according to the present invention include fragments or variants of the genomic DNA inserts of the BAC clones described that retain the ability to segregate during mitotic or meiotic division. Variants of such sequences include artificially produced modifications and modifications produced via passaging through one or more bacteria, plant or other host cells. Vectors comprising 1, 2, 3, 4, 5, 6, 7, 8, 9, 10, 15 or 20 or more of the elements contained in any of the exemplary vectors are also contemplated.

The invention specifically contemplates the alternative use of fragments or variants (mutants) of any of the nucleic acids described herein that retain the desired activity, including nucleic acids that function as centromeres, nucleic acids that function as promoters or other regulatory control sequences, or exogenous nucleic acids. Variants can have one or more additions, substitutions or deletions of nucleotides within the original nucleotide sequence. Variants include nucleic acid sequences that are at least 50, 55, 60, 65, 70, 75, 80, 81, 82, 83, 84, 85, 86, 87, 88, 89, 90, 91, 92, 93, 94, 95, 96, 97, 98, or 99% identical to the original nucleic acid sequence. Variants also include nucleic acid sequences that hybridize under low, medium, high or very high stringency conditions to the original nucleic acid sequence.

The comparison of sequences and determination of percent identity between two nucleotide sequences can be accomplished using a mathematical algorithm. For example, the percent identity between two amino acid sequences can be determined using the Needleman and Wunsch algorithm that has been incorporated into the GAP program in the GCG software package (Needleman and Wunsch 1970), using either a Blossum 62 matrix or a PAM250 matrix. Parameters are set so as to maximize the percent identity.

“Hybridizes under low stringency, medium stringency, and high stringency conditions” describes conditions for hybridization and washing. Hybridization is a well-known technique (Ausubel 1987). Low stringency hybridization conditions means, for example, hybridization in 6× sodium chloride/sodium citrate (SSC) at about 45° C., followed by two washes in 0.5×SSC, 0.1% SDS, at least at 50° C.; medium stringency hybridization conditions means, for example, hybridization in 6×SSC at about 45° C., followed by one or more washes in 0.2×SSC, 0.1%) SDS at 55° C.; and high stringency hybridization conditions means, for example, hybridization in 6×SSC at about 45° C., followed by one or more washes in 0.2×SSC, 0.1% SDS at 65° C. Another non limiting example of stringent hybridization conditions are hybridization in a high salt buffer comprising 6×SSC, 50 mM Tris HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. Another non limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Another non limiting example of low stringency hybridization conditions are hybridization in 35% formamide, 5×SSC, 50 mM Tris HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency that may be used are well known in the art (e.g., as employed for cross species hybridizations).

MC Sequence Content and Structure

Plant-expressed genes from non-plant sources can be modified to accommodate plant codon usage, to insert preferred motifs near the translation initiation ATG codon, to remove sequences recognized in plants as 5′ or 3′ splice sites, or to better reflect plant GC/AT content. Plant genes typically have a GC content of more than 35%, and coding sequences that are rich in A and T nucleotides can be problematic. For example, ATTTA motifs can destabilize mRNA; plant polyadenylation signals such as AATAAA at inappropriate positions within the message can cause premature truncation of transcription; and monocotyledons can recognize AT-rich sequences as splice sites.

Each exogenous nucleic acid or plant-expressed gene can include a promoter, a coding region and a terminator sequence, that can be separated from each other by restriction endonucleasc sites or recombination sites or both. Genes can also include introns, that can be present in any number and at any position within the transcribed portion of the gene, including the 5′ untranslated sequence, the coding region and the 3′ untranslated sequence. Introns can be natural plant introns derived from any plant, or artificial introns based on the splice site consensus that has been defined for plant species. Some intron sequences have been shown to enhance expression in plants. Optionally the exogenous nucleic acid can include a plant transcriptional terminator, non-translated leader sequences derived from viruses that enhance expression, a minimal promoter, or a signal sequence controlling the targeting of gene products to plant compartments or organelles.

The coding regions of the genes can encode any protein, including visible marker genes (for example, fluorescent protein genes, other genes conferring a visible phenotype), other screenable or selectable marker genes (for example, conferring resistance to antibiotics, herbicides or other toxic compounds, or encoding a protein that confers a growth advantage to the cell expressing the protein) or genes that confer some commercial or agronomic value to the adchromosomal plant. Multiple genes can be placed on the same MC vector. The genes can be separated from each other by restriction endonuclease sites, homing endonuclease sites, recombination sites or any combinations thereof. Any number of genes can be present. Genes on a MC can be in any orientation with respect to one another and with respect to the other elements of the MC (e.g. the centromere).

The MC vector can also contain a bacterial plasmid backbone for propagation of the plasmid in bacteria such as E. coli, A. tumefaciens, or A. rhizogenes. The plasmid backbone can be that of a low-copy vector or mid to high level copy backbone. This backbone can contain the replicon of the F′ plasmid of E. coli. However, other plasmid replicons, such as the bacteriophage P1 replicon, or other low-copy plasmid systems, such as the RK2 replication origin, can also be used. The backbone can include one or several antibiotic-resistance genes conferring resistance to a specific antibiotic to the bacterial cell in that the plasmid is present. Examples of bacterial antibiotic-resistance genes include kanamycin-, ampicillin-, chloramphenicol-, streptomycin-, spectinomycin-, tetracycline- and gentamycin-resistance genes. The backbone can also be designed so that it can be excised from the MC prior to delivery to a plant cell. The use of flanking restriction enzyme sites or flanking site-specific recombination sites are both useful for constructing a removable backbone.

The MC vector can also contain plant telomeres. An exemplary telomere sequence is tttaggg (SEQ ID NO:98) or its complement. Telomeres stabilize the ends of linear chromosomes and facilitate the complete replication of the extreme termini of the DNA molecule (Ausubel, 1987; Richards and Ausubel, 1988).

Additionally, the MC vector can contain “stuffer DNA” sequences that serve to separate the various components on the MC. Stuffer DNA can be of any origin, synthetic, prokaryotic or eukaryotic, and from any genome or species, plant, animal, microbe or organelle. Stuffer DNA can range from 10 bp, 20 bp, 30 bp, 40 bp, 50 bp, 60 bp, 70 bp, 80 bp, 90 bp, 100 bp, 150 bp, 200 bp, 300 bp, 400 bp 500 bp, 750 bp, 1000 bp, 2000 bp, 5000 bp, 10 kb, 20 kb, 50 kb, 75 kb, 1 Mb to 10 Mb in length and can be repetitive in sequence, with unit repeats from 10 bp to 1 Mb. Examples of repetitive sequences that can be used as stuffer DNAs include rDNA, satellite repeats, retroelements, transposons, pseudogenes, transcribed genes, microsatellites, tDNA genes, short sequence repeats and combinations thereof. Alternatively, stuffer DNA can consist of unique, non-repetitive DNA of any origin or sequence. The stuffer sequences can also include DNA with the ability to form boundary domains, such as scaffold attachment regions (SARs) or matrix attachment regions (MARs). Stuffer DNA can be entirely synthetic, composed of random sequence, having any base composition, or any A/T or G/C content.

In one embodiment of the invention, the MC has a circular structure without telomeres. In another embodiment, the MC has a circular structure with telomeres. In a third embodiment, the MC has a linear structure with telomeres. A “linear” structure can be generated by cutting a circular MC that contains telomeres with an endonuclease(s), that expoes the telomeres at the ends of the resultant linear nucleic acid molecule that contains all of the sequence contained in the original, closed construct. A variant of this strategy is to separate two telomere elements with an antibiotic-resistance gene that is also excised upon linearization. In a fourth embodiment of the invention, the telomeres could be placed in such a manner that the bacterial replicon, backbone sequences, antibiotic-resistance genes and any other sequences of bacterial origin and present for the purposes of propagation of the MC in bacteria, can be removed from the plant-expressed genes, the centromere, telomeres, and other sequences by cutting the structure with an endonuclease(s). When removing intervening sequences to expose telomere elements during linearization site-specific recombination systems can be used instead of endoculeases. These linearization techniques result in a MC from which much of, or preferably all, bacterial sequences have been removed. In this embodiment, bacterial sequence present between or among the plant-expressed genes or other MC sequences are excised prior to removal of the remaining bacterial sequences by cutting the MC with a homing endonuclease, and re-ligating the structure or by using site-specific recombination systems. Particularly useful endonucleases are those that are present only at the desired linearization site (unique), including homing endonuclease sites. Alternatively, the endonucleases and their sites can be replaced with any specific DNA cutting mechanism and its specific recognition site, such as a rare-cutting endonuclease or recombinase and its specific recognition site, as long as that site is present in the MC.

Various structural configurations of the MC elements are possible. A centromere can be placed on a MC either between genes or outside a cluster of genes next to a telomere. Stuffer DNAs can be combined with these configurations including stuffer sequences placed inside the telomeres, around the centromere between genes or any combination thereof. Thus, a large number of alternative MC structures are possible, depending on the relative placement of centromere DNA, genes, stuffer DNAs, bacterial sequences, telomeres, and other sequences. Such variations in architecture are possible both for linear and for circular MCs.

Exemplary Centromere Components

In one embodiment, the centromere contains n copies of a repeated nucleotide sequence, such as that of SEQ ID NOs:90-92 or of SEQ ID NOs:94-97, wherein n is at least 2. In another embodiment, the centromere contains n copies of interdigitated repeats. An interdigitated repeat is a DNA sequence that consists of two distinct repetitive elements that combine to create a unique permutation. Potentially any number of repeat copies capable of physically being placed on the recombinant construct could be included on the construct, including about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 120, 140, 150, 200, 300, 400, 500, 750, 1,000, 1,500, 2,000, 3,000, 5,000, 7,500, 10,000, 20,000, 30,000, 40,000, 50,000, 60,000, 70,000, 80,000, 90,000 and about 100,000, including all ranges in-between such copy numbers. Moreover, the copies can vary from each other, such as is commonly observed in naturally occurring centromeres. The length of the repeat can vary, but will preferably range from about 20 bp to about 360 bp, from about 20 bp to about 250 bp, from about 50 bp to about 225 bp, from about 75 bp to about 210 bp, such as a 92 bp repeat and a 97 bp repeat, from about 100 bp to about 205 bp, from about 125 bp to about 200 bp, from about 150 bp to about 195 bp, from about 160 bp to about 190 and from about 170 bp to about 185 bp including about 180 bp. The length of the repeat can also be about 100 to 210 bp; such as 100, 194, and 210 bp. The length of the repeat can also include larger sequences, from about 300 bp to about 10 kb, from about 1 kb to 9 kb, from about 2 kb to about 8 kb, from about 3 kb to about 7 kb, from about 4 kb to about 8 kb, including, for example, 982 bp, 2836 bp, 5788 bp and 8308 bp.

Modification of Centromeres Isolated from Native Plant Genome

Modification and changes can be made in the centromeric DNA segments of the current invention and still obtain a functional molecule with desirable characteristics. The following is a discussion based upon changing the nucleic acids of a centromere to create an equivalent, or even an improved, second generation molecule.

In particular embodiments of the invention, mutated centromeric sequences are contemplated to be useful for increasing the utility of the centromere. It is specifically contemplated that the function of the centromeres of the current invention can be based in part or in whole upon the secondary structure of the DNA sequences of the centromere, modification of the DNA with methyl groups or other adducts, and/or the proteins that interact with the centromere. By changing the DNA sequence of the centromere, one can alter the affinity of one or more centromere-associated protein(s) for the centromere and/or the secondary structure or modification of the centromeric sequences, thereby changing the activity of the centromere. Alternatively, changes can be made in the centromeres that do not affect the activity of the centromere. Changes in the centromeric sequences that reduce the size of the DNA segment needed to confer centromere activity are particularly useful, as are changes that increase the fidelity with that the centromere is transmitted during mitosis and meiosis.

Modification of Centromeres by Passage Through Bacteria, Plant or Other Hosts or Processes

In the methods of the present invention, the resulting MC DNA sequence can also be a derivative of the parental clone or centromere clone having substitutions, deletions, insertions, duplications and/or rearrangements of one or more nucleotides in the nucleic acid sequence. Such nucleotide mutations can occur individually or consecutively in stretches of 1, 2, 3, 4, 5, 6, 7, 8, 9, 10, 20, 40, 50, 60, 70, 80, 90, 100, 150, 200, 300, 400, 500, 800, 1000, 2000, 4000, 8000, 10000, 50000, 100000, and about 200000, including all ranges in-between. Variations of MCs can arise through passage of MCs through various hosts including virus, bacteria, yeast, plant or other prokaryotic or eukaryotic organism and can occur through passage of multiple hosts or individual host. Variations can also occur by replicating the MC in vitro. Variations can also be specifically engineered into the MC using standard molecular biology techniques.

### D. Exemplary Exogenous Nucleic Acids Including Plant-Expressed Genes and Regulatory Elements

Of particular interest in the present invention are exogenous nucleic acids that when introduced into plants alter the phenotype of the plant, a plant organ, plant tissue, or portion of the plant. Such exogenous nucleic acids can be delivered on MCs. Exemplary exogenous nucleic acids encode polypeptides involved in one or more important biological properties in plants. Other exemplary exogenous nucleic acids alter expression of exogenous or endogenous genes, either increasing or decreasing expression, optionally in response to a specific signal or stimulus. Other exemplary exogenous nucleic acids encode polypeptides that produce a trait in the plant that is not found in the plant before the introduction of the exogenous nucleic acid.

One of the major purposes of transformation of crop plants is to add some commercially desirable, agronomically important traits to the plant. Such traits include herbicide resistance or tolerance; insect (pest) resistance or tolerance; nematode resistance, disease resistance or tolerance (viral, bacterial, fungal, or other pathogens); stress tolerance and/or resistance, as exemplified by resistance or tolerance to drought, heat, chilling, freezing, excessive moisture, salt stress, mechanical stress, extreme acidity, alkalinity, toxins, UV light, ionizing radiation or oxidative stress; increased yields, whether in quantity or quality; enhanced or altered nutrient acquisition and enhanced or altered metabolic efficiency; enhanced or altered nutritional content (including altered gossypol levels) and makeup of plant tissues used for food, feed, fiber or processing; physical appearance; male sterility; drydown; standability; prolificacy; altered geographical range; altered day-length tolerance; starch quantity and quality; oil quantity and quality; protein quality and quantity; amino acid composition; modified chemical production; altered pharmaceutical or nutraceutical properties; altered bioremediation properties; increased biomass; altered growth rate; altered fitness; altered biodegradability; altered CO₂ fixation; presence of bioindicator activity; altered digestibility by humans or animals; altered allergenicity; altered mating characteristics; altered pollen dispersal; improved environmental impact; altered nitrogen fixation capability; the production of a pharmaceutically active protein; the production of a small molecule with medicinal properties; the production of a chemical including those with industrial utility; the production of fibers including those used in making clothing, towels, bedding, wall coverings, upholstery, draperies, textiles, yarn, thread, wicks, string, paper, medical bandages, cotton balls, cotton batting, cotton swabs, cotton wool, gauze, tampons and other femine hygine products, cellulose products (e.g. rayon, plastics, photographic film, and cellophane), tarps and other industrial materials; the production of nutraceuticals, food additives, carbohydrates, RNAs, lipids, fuels, dyes, pigments, vitamins, scents, flavors, vaccines, antibodies, hormones, and the like; and alterations in plant architecture or development, including changes in developmental timing, photosynthesis, signal transduction, cell growth, reproduction, or differentiation. Additionally one could create a library of an entire genome from any organism or organelle including mammals, plants, microbes, fungi, or bacteria, represented on MCs.

A modified plant can exhibit increased or decreased expression or accumulation of a product that can be a natural product of the plant or a new or altered product. Examples of products include enzymes, RNA molecules, nutritional proteins, structural proteins, amino acids, lipids, fatty acids, polysaccharides, sugars, alcohols, alkaloids, carotenoids, propanoids, phenylpropanoids, terpenoids, steroids, flavonoids, phenolics, anthocyanins, pigments, vitamins or plant hormones. The modified plant can have enhanced or diminished requirements for light, water, nitrogen, or trace elements. Modified plant can also have an enhanced ability to capture or fix nitrogen from its environment. Modified plants are enriched for an essential amino acid as a proportion of a protein fraction of the plant. The protein fraction can be, for example, total seed protein, soluble protein, insoluble protein, water-extractable protein, and lipid-associated protein. The modification can include overexpression, underexpression, antisense modulation, sense suppression, induciblc expression, inducible repression, or inducible modulation of a gene.

A brief summary of exemplary improved properties and polypeptides of interest for either increased or decreased expression is provided below.

Herbicide Resistance

A herbicide resistance (or tolerance) trait is a characteristic of a modified plant that is resistant to dosages of an herbicide that is typically lethal to a non-modified plant. Exemplary herbicides useful in a plant include members of the following families of herbicides: Acetamide, Amide, Arylaminopropionic acid, Aryloxyphenoxy-propionate, Benzamide, Benzofuran, Benzoic acid, Benzothiadiazinone, Bialaphos, Bipyridylium, Carbamate, Chloroacetamide, Chloro-Carbonic-acid, Chlorophenoxy acid, Dinitroaniline, Dinitrophenol, Diphenylether, Glufosinate, Glycine, Glyphosate, Imidazolinone, Isoxazole, Isoxazolidinone, Nitrile, N-phenylphthalimide, Organoarsenical, Oxadiazole, Oxazolidinedione, Oxyacetamide, Oxynil, Phenoxy-carboxylic-acid, Phenyl-carbamate, Phenylpyrazole, Phenylpyrazoline, Phenyl-pyridazine, Phosphinic acid, Phosphinothricin, Phosphoroamidate, Phosphorodithioate, Phthalamate Semicarbazone, Pyrazole, Pyrazolium, Pyridazinone, Pyridine, Pyridine carboxylic acid, Pyridinecarboxamide, Pyrimidindione, Pyrimidinyl(thio)benzoate, Quinoline carboxylic acid, Sulfonamide, Sulfonylaminocarbonyl-triazolinone, Sulfonylurea, Tetrazolinone, Thiadiazole, Thiocarbamate, Triazine, Triazinone, Triazole, Triazolinone, Triazolocarboxamide, Triazolopyrimidine, Triketone, Uracil or Urea herbicides. MCs carrying resistance genes to one or more of these herbicides are anticipated. Other herbicide resistance genes would be useful as would combinations of herbicide resistance genes on the same MC.

The genes encoding phosphinothricin acetyltransferase (bar), glyphosate tolerant EPSP synthase genes, glyphosate acetyltransferase, the glyphosate degradative enzyme gene gox encoding glyphosate oxidoreductase, deh (encoding a dehalogcnase enzyme that inactivates dalapon), herbicide resistant (e.g., sulfonylurea and imidazolinone) acetolactate synthase, and bxn genes (encoding a nitrilase enzyme that degrades bromoxynil) are good examples of herbicide resistant genes for use in transformation. The bar gene codes for an enzyme, phosphinothricin acetyltransferase (PAT), that inactivates the herbicide phosphinothricin and prevents this compound from inhibiting glutamine synthetase enzymes. The enzyme 5-enolpyruvylshikimate-3-phosphate synthase (EPSP Synthase), is normally inhibited by the herbicide N (phosphonomethyl) glycine (glyphosate). However, genes are known that encode glyphosate resistant EPSP synthase enzymes. These genes are particularly contemplated for use in plant transformation. The deh gene encodes the enzyme dalapon dehalogenase and confers resistance to the herbicide dalapon. The bxn gene codes for a specific nitrilase enzyme that converts bromoxynil to a non herbicidal degradation product. The glyphosate acetyl transferase gene inactivates the herbicide glyphosate and prevents this compound from inhibiting EPSP synthase.

Polypeptides that can produce plants having tolerance to plant herbicides include polypeptides involved in the shikimate pathway, that are of interest for providing glyphosate tolerant plants. Such polypeptides include polypeptides involved in biosynthesis of chorismate, phenylalanine, tyrosine and tryptophan.

Insect Resistance

Potential insect resistance (or tolerance) genes that can be introduced include Bacillus thuringiensis crystal toxin genes or Bt genes (Watrud 1985). Bt genes can provide resistance to lepidopteran or coleopteran pests such as European Corn Borer (ECB). Examples of Bt toxin genes include the CryIA(b) and CryIA(c) genes. Endotoxin genes from other species of B. thuringiensis that affect insect growth or development also can be used.

Bt genes for use in the MCs can be modified to increase expression in plants. Means for preparing synthetic genes are well known in the art. Examples of modified Bt toxin genes include a synthetic Bt CryIA(b) gene (Perlak et al., 1991), and the synthetic CryIA(c) gene termed 1800b (WO95/06128). Some examples of other Bt toxin genes known to those of skill in the art are given in Table 1.

TABLE 1
Examples of Bt toxin genes

| Gene | GenBank Accession |
|---|---|
| Cry1Aa | M11250 |
| Cry1Ab | M13898 |
| Cry1Ac | MI 1068 |
| Cry 1 Ad | M73250 |
| Cry1Ae | M65252 |
| Cry1Ba | X06711 |
| Cry1Bb | L32020 |
| Cry1Bc | Z46442 |
| Cry1Bd | U70726 |
| Cry1Ca | X07518 |
| Cry1Cb | M97880 |
| Cry1Da | X54160 |
| Cry1Db | Z22511 |
| Cry1Ea | X53985 |
| Cry1Eb | M73253 |
| Cry1Fa | M63897 |
| Cry1Fb | Z22512 |
| Cry1Ga | Z22510 |
| Cry1Gb | U70725 |
| Cry1Ha | Z22513 |
| Cry1Hb | U35780 |
| Cry1Ha | X62821 |
| Cry1Ib | U07642 |
| Cry1Ja | L32019 |
| Cry1Jb | U31527 |
| Cry1K | U28801 |
| Cry2Aa | M31738 |
| Cry2Ab | M23724 |
| Cry2Ac | X57252 |
| Cry3A | M22472 |
| Cry3Ba | X17123 |
| Cry3Bb | M89794 |
| Cry3C | X59797 |
| Cry4A | Y00423 |
| Cry4B | X07423 |
| Cry5Aa | L07025 |
| Cry5Ab | L07026 |
| Cry6A | L07022 |
| Cry6B | L07024 |
| Cry7Aa | M64478 |
| Cry7Ab | U04367 |
| CrySA | U04364 |
| CrySB | U04365 |
| Cry8C | U04366 |
| Cry9A | X58120 |
| Cry9B | X75019 |
| Cry9C | Z37527 |
| Cry10A | M12662 |
| Cry11A | M31737 |
| Cry12B | X86902 |
| Cry12A | L07027 |
| Cry13A | L07023 |
| Cry14A | U13955 |
| Cry15A | M76442 |
| Cry16A | X94146 |
| Cry17A | X99478 |
| Cry18A | X99049 |
| Cry19A | Y0S920 |
| Cyt1Aa | X03182 |
| Cyt1Ab | X98793 |
| Cyt2A | Z14147 |
| Cyt2B | U52043 |

Protease inhibitors also can provide insect resistance (Johnson et al., 1989) and can also be used. The use of a protease inhibitor II gene, pinII, from tomato or potato is particularly useful, especially when used in combination with a Bt toxin gene, because the combined effect has synergistic insecticidal activity. Other genes that encode inhibitors of the insect's digestive system, or those that encode enzymes or co-factors that facilitate the production of inhibitors, are also useful, such as oryzacystatin and amylase inhibitors from wheat and barley. Several amylase inhibitor genes have been isolated from plants and some have been introduced as exogenous nucleic acids, conferring an insect resistant phenotype that is useful (Chrispeels, Sadava et al. 2003).

Genes encoding lectins can confer additional or alternative insecticide properties. Lectins are multivalent carbohydrate binding proteins that have the ability to agglutinate red blood cells from a range of species. Lectins have been identified recently as insecticidal agents with activity against weevils, ECB and rootworm (Murdock et al., Phytochemistry, 29:85-89, 1990, (Murdock, Huesing et al. 1990; Czapla and Lang 1991). Lectin genes contemplated to be useful include, for example, barley and wheat germ agglutinin (WGA) and rice lectins (Gatehouse, Dewey et al. 1984).

Genes controlling the production of large or small polypeptides active against insects when introduced into the insect pests, such as lytic peptides, peptide hormones and toxins and venoms can also be delivered on MCs. For example, the expression of juvenile hormone esterase, directed towards specific insect pests, can result in insecticidal activity, or halt metamorphosis (Hammock, Bonning et al. 1990).

Genes that encode enzymes that affect the integrity of the insect cuticle can also be used. Such genes include those encoding, e.g., chitinase, proteases, lipases and also genes for the production of nikkomycin, a compound that inhibits chitin synthesis, the introduction of any one of these can produce insect resistant plants. Genes that code for activities that affect insect molting, such as those effecting the production of ecdysteroid UDP glucosyltransferase, can also be used.

Genes that code for enzymes that facilitate the production of compounds that reduce the nutritional quality of the host plant to insect pests can also be used. Insecticidal activity can be conferred on a plant by altering its sterol composition. Alterations in plant sterol composition by expression of genes that directly promote the production of undesirable sterols or those that convert desirable sterols into undesirable forms can have a negative effect on insect growth and/or development. Lipoxygenases are naturally occurring plant enzymes that have been shown to exhibit anti nutritional effects on insects and to reduce the nutritional quality of their diet.

Tripsacum dactyloides is a species of grass that is resistant to certain insects, including corn root worm. Genes isolated from Tripsacum that confers insect resistance can be delivered on MCs.

Further, genes encoding proteins characterized as having potential insecticidal activity also can be used as exogenous nucleic acids. Such genes include, for example, the cowpea trypsin inhibitor (CpTI; (Hilder, Gatehouse et al. 1987)) that can be used as a rootworm deterrent; genes encoding avermectin ((Campbell, 1989; Ikeda et al., 1987) that can prove particularly useful as a corn rootworm deterrent; ribosome inactivating protein genes; and genes that regulate plant structures. Modified plants including anti-insect antibody genes and genes that code for enzymes that can convert a non toxic insecticide (pro insecticide) applied to the outside of the plant into an insecticide inside the plant also are contemplated.

Polypeptides that can improve plant tolerance to the effects of plant pests or pathogens include proteases, polypeptides involved in anthocyanin biosynthesis, polypeptides involved in cell wall metabolism, including cellulases, glucosidases, pectin methyl esterase, pectinase, polygalacturonase, chitinase, chitosanase, and cellulose synthase, and polypeptides involved in biosynthesis of terpenoids or indole for production of bioactive metabolites to provide defense against herbivorous insects.

Vegetative Insecticidal Proteins (VIP) are a class of proteins originally found to be produced in the vegetative growth phase of the bacterium, Bacillus cereus, but do have a spectrum of insect lethality similar to the insecticidal genes found in strains of Bacillus thuriengensis. Both the vip1a and vip3A genes have been isolated and have demonstrated insect toxicity (Chrispeels, Sadava et al. 2003).

In cotton, genes that control the following pests are advantageous, including: Agrotis ipsilon (black cutworm), Anthonomous grandis grandis (boll weevil), Aphis craccivora (cowpea aphid), Aphis gossypii (cotton aphid), Creontiades dilutes, Dysdercus Koenigii (cotton stainer), Feltia subterranean (granulate cutworm), Frankliniella exigua (thrips), Frankliniella fusca (tobacco thrips), Frankliniella tritici (flower thrips), Franliniella schultzei (thrips), Helicoverpa armigera, Helicoverpa punctigera, Heliothis virescens (tobacco budworm), Heliothis zea (bollworm), Lepidoptera larvae, Lygus lineolaris (Tarnished Plant Bug), Peridroma saucia (variegated cutworm), Sericothrips variabilis (soybean thrips), Spodoptera exigua (beet armyworm), Spodoptera frugiperda (fall armyworm), Spodoptera ornithogalli (yellow-striped armyworm), Tetranchus spider mites, Tetranchus ludeni, Tetranchus urticae, Tetranhcus lambi, Tetranychus urticae (two-spotted spider mite), Thrips tabaci (onion thrips), and Trichoplusia ni (cabbage looper).

Environment or Stress Resistance

Improvement of a plant's ability to tolerate various environmental stresses such as drought, excess moisture, chilling, freezing, high temperature, salt, and oxidative stress, can also be effected through expression of exogenous genes. Benefits can be realized in terms of increased resistance to freezing temperatures through the introduction of an antifreeze protein. Improved chilling tolerance also can be conferred through increased expression of glycerol 3 phosphate acetyltransferase in chloroplasts (Wolter, Schmidt et al. 1992). Resistance to oxidative stress (often exacerbated by conditions such as chilling temperatures in combination with high light intensities) can be conferred by expression of superoxide dismutase, and can be improved by glutathione reductase (Bowler, Montagu et al. 1982). Such strategies can allow for tolerance to freezing in newly emerged fields as well as extending later maturity higher yielding varieties to earlier relative maturity zones.

The expression of novel genes that favorably effect plant water content, total water potential, osmotic potential, or turgor can enhance the ability of the plant to tolerate drought. “Drought resistance” and “drought tolerance” refer to a plant's increased resistance or tolerance to stress induced by a reduction in water availability, as compared to normal circumstances, and the ability of the plant to function and survive in lower water environments. The expression of genes encoding for the biosynthesis of osmotically active solutes, such as polyol compounds, can impart protection against drought. Within this class are genes encoding for mannitol-1 phosphate dehydrogenase (Lee and Saier 1983) and trehalose-6-phosphate synthase (Kaasen et al., 1992; Tarczynski et al., 1993).

The efficacy of other metabolites can protect enzyme function (e.g., alanopine or propionic acid) or membrane integrity (e.g., alanopine) (Loomis, Carpenter et al. 1989), and therefore expression of genes encoding for the biosynthesis of these compounds can confer drought resistance in a manner similar to or complimentary to mannitol. Other examples of naturally occurring metabolites that are osmotically active and/or provide some direct protective effect during drought and/or desiccation include fructose, erythritol, sorbitol, dulcitol, glucosylglycerol, sucrose, stachyose, raffinose, proline, glycine betaine, ononitol and pinitol.

The expression of specific proteins also can increase drought tolerance. The Type II Late Embryogenic Abundant (LEA) proteins (dehydrin type) have been implicated in drought and/or desiccation tolerance in vegetative plant parts (e.g., (Yamaguchi-Shinozaki, Kozumi et al. 1992)). Expression of a Type III LEA (HVA 1) in tobacco was found to influence plant height, maturity and drought tolerance (Fitzpatrick 1989). In rice, expression of the HVA 1 gene influenced tolerance to water deficit and salinity (Xu, Duan et al. 1996). Expression of structural genes from any of the three LEA groups can therefore confer drought tolerance. Other types of proteins induced during water stress include thiol proteases, aldolases or transmembrane transporters (Guerrero, Jones et al. 1990), that can confer various protective and/or repair type functions during drought stress. It also is contemplated that genes that effect lipid biosynthesis can also be useful in conferring drought resistance on the plant.

Many of these genes also improve freezing tolerance (or resistance); the physical stresses incurred during freezing and drought are similar in nature and can be mitigated in similar fashion. Benefit can be conferred via constitutive expression of these genes, but a useful means of expressing these genes can be through the use of a turgor induced promoter (Guerrero, Jones et al. 1990). Spatial and temporal expression patterns of these genes can enable plants to better withstand stress.

Expression of genes involved with specific morphological traits that allow for increased water extractions from drying soil can also be used. For example, introduction and expression of genes that alter root characteristics can enhance water uptake. Expression of genes that enhance reproductive fitness during times of stress are also useful, such as expression of genes that improve the synchrony of pollen shed and receptiveness of the female flower parts, e.g., silks. Expression of genes that minimize flower abortion during times of stress would increase the amount of cotton to be harvested and hence be of value.

Given the overall role of water in determining yield, it is contemplated that enabling plants to use water more efficiently, through the introduction and expression of exogenous genes, can improve overall performance even when soil water availability is not limiting. By introducing genes that improve the ability of plants to maximize water usage across a full range of stresses relating to water availability, yield stability or consistency of yield performance can be realized.

Polypeptides that can improve stress tolerance under a variety of stress conditions include those polypeptides involved in gene regulation, such as serine/threonine-protein kinases, MAP kinases, MAP kinase kinases, and MAP kinase kinase kinases; polypeptides that act as receptors for signal transduction and regulation, such as receptor protein kinases; intracellular signaling proteins, such as protein phosphatases, GTP binding proteins, and phospholipid signaling proteins; polypeptides involved in arginine biosynthesis; polypeptides involved in ATP metabolism, including for example ATPase, adenylate transporters, and polypeptides involved in ATP synthesis and transport; polypeptides involved in glycine betaine, jasmonic acid, flavonoid or steroid biosynthesis; and hemoglobin. Enhanced or reduced activity of such polypeptides in modified plants will provide changes in the ability of a plant to respond to a variety of environmental stresses, such as chemical stress, drought stress and pest stress.

Other polypeptides that can improve plant tolerance to cold or freezing temperatures include polypeptides involved in biosynthesis of trehalose or raffinose, polypeptides encoded by cold induced genes, fatty acyl desaturases and other polypeptides involved in glycerolipid or membrane lipid biosynthesis, that find use in modification of membrane fatty acid composition, alternative oxidase, calcium-dependent protein kinases, LEA proteins or uncoupling protein.

Other polypeptides that can improve plant tolerance to heat include polypeptides involved in biosynthesis of trehalose, polypeptides involved in glycerolipid biosynthesis or membrane lipid metabolism (for altering membrane fatty acid composition), heat shock proteins or mitochondrial NDK.

Other polypeptides that can improve tolerance to extreme osmotic conditions include polypeptides involved in proline biosynthesis.

Other polypeptides that can improve plant tolerance to drought conditions include aquaporins, polypeptides involved in biosynthesis of trehalose or wax, LEA proteins or invertase.

Disease Resistance

Increased resistance (or tolerance) to diseases caused by viruses, viroids, bacteria, fungi and nematodes, as well as mycotoxin-producing organisms, can be realized through the introduction of exogenous genes. Resistance can be affected through suppression of endogenous factors that encourage disease-causing interactions, expression of exogenous factors that are toxic to or otherwise provide protection from pathogens, or expression of factors that enhance the plant's own defense responses.

Resistance to viruses can be produced through expression of novel genes. For example, expression of a viral coat protein in a modified plant can impart resistance to infection of the plant by that virus and perhaps other closely related viruses (Abel, Nelson et al. 1986). Expression of antisense genes targeted at essential viral functions can also impart resistance to viruses. Further, resistance to viruses can be realized through other approaches, including the use of satellite viruses.

Increased resistance to diseases caused by bacteria and fungi can be realized through introduction of exogenous genes. “Peptide antibiotics,” pathogenesis related (PR) proteins, toxin resistance, or proteins effecting host pathogen interactions such as morphological characteristics are useful. Peptide antibiotic genes are induced following pathogen attack on a host plant and have been divided into at least 5 classes of proteins. Included amongst the PR proteins are beta 1,3 glucanases, chitinases, and osmotin and other proteins that are believed to function in plant resistance to disease organisms. Other genes have been identified that have antifungal properties, e.g., UDA (stinging nettle lectin), or hevein. Certain plant diseases are caused by the production of phytotoxins; introduction of an exogenousgene that encodes an enzyme capable of degrading or otherwise inactivating the phytotoxin can confer resistance. Exogenous genes that alter the interactions between host plant and pathogen can be useful to reduce the ability of the disease organism to invade the tissues of the host plant, e.g., an increase in the waxiness of the leaf cuticle or other morphological characteristics.

Polypeptides useful for imparting improved disease responses to plants include polypeptides encoded by cercosporin-induced genes, antifungal proteins and proteins encoded by R-genes or SAR genes.

Agronomically important diseases caused by fungal phytopathogens include anthracnose, arcolate mildew, boll rot, charcoal rot, fusarium wilt, rust, sclerotium stem rot, black root rot, Ascochyta blight or ashen spot, leaf blight and spot, wilt, sheath blight, stem canker, or root rot.

Exemplary plant viruses include tobacco or cucumber mosaic virus, ringspot virus, necrosis virus, and maize dwarf mosaic virus. Specific animal, fungal, protist, bacterial, and viral pathogens of major crops include, in cotton, Verticillium dahliae, Fusarium moniliforme, Fusarium roseum, Fusarium oxysporum, Phomopsis sp., Cladosporium sp., Alternaria alternate, Alternaria macrospora, Glomerella gossypii, Glomerella cingulata, Hoplolaimus columbus, Ascochyta gossypii, Phyophthora capsici, Diplodia gossypina, Mycosphaerella areola, Salmonia malachrae, Leveillula taurica, Thielaviopsis basicola, Chalara elegans, Lasiodiplodia theobromae, Phymatotrichum omnivorum, Sclerotium rolfsii, Macrophomina phaseolina, Puccinia cacabata, Puccinia schedonnardi, Phoma exigua, Phakopsora gossypii, Pythium aphanidermatum, Pythium ultimum, Pythium debaryanum, Pythium heterothallicum, Pythium irregulare, Pythium polytylum, Pythium splendens, Pythium sylvaticum, Pythiumultimum, Verticullium dahliae, Colletotrichum gossypii, Rhizoctonia solani, Rhizopus oryzae, Geminiviruses such as leaf crumple virus and leaf curl virus, Cotton mosaic virus, Rotylenchulus reniformis (Reniform nematode), Meloidogyne incognita (Root knot nematode), Belonolaimus longicaudatus (sting nematode), Cochliobolus spicifer, Myrothecium roridum, Nigrospora oryzae, Xanthomonas campestris pv malvacearum, Nematospora spp. Aspergillus sp., Phomopsis sp., Cercospora sp., Stemphyllium sp., Phymatotrichopsis omnivore.

Plant Agronomic Characteristics

Two of the factors determining where crop plants can be grown are the average daily temperature during the growing season and the length of time between frosts. Within the areas where it is possible to grow a particular crop, there are varying limitations on the maximal time it is allowed to grow to maturity and be harvested. For example, a variety to be grown in a particular area is selected for its ability to mature and dry down to harvestable moisture content within the required period of time with maximum possible yield. Therefore, crops of varying maturities are developed for different growing locations. Apart from the need to dry down sufficiently to permit harvest, it is desirable to have maximal drying take place in the field to minimize the amount of energy required for additional drying post harvest. Also, the more readily a product can dry down, the more time there is available for growth and mature development. Genes that influence maturity and/or dry down can be identified and introduced into plant lines using transformation techniques to create new varieties adapted to different growing locations or the same growing location, but having improved yield to moisture ratio at harvest. Expression of genes that are involved in regulation of plant development can be especially useful.

IGenes can be introduced into plants that can improve standability and other plant growth characteristics. Expression of exogenous genes in plants that confer stronger stems, improved root systems, or prevent or reduce boll loss or shattering are especially attractive to farmers. Iintroduction and expression of genes that increase the total amount of photoassimilate available by, for example, increasing light distribution and/or interception is advantageous. In addition, the expression of genes that increase the efficiency of photosynthesis and/or the leaf canopy can further increase gains in productivity. Expression of a phytochrome gene in crop plants can be advantageous. Expression of such a gene can reduce apical dominance, confer semidwarfism on a plant, or increase shade tolerance (U.S. Pat. No. 5,268,526). Such approaches would allow for increased plant populations in the field.

Nutrient Utilization

The ability to utilize available nutrients can be a limiting factor in growth of crop plants. Nutrient uptake, ability to tolerate pH extremes, nutrient mobilization through the plant, nutrient storage pools, and availability for metabolic activities can be altered by the introduction of exogenous genes. An increase in the activity of, for example, an enzyme that is normally present in the plant and involved in nutrient utilization can increase the availability of a nutrient or decrease the availability of an antinutritive factor. An example of such an enzyme is phytase. Enhanced nitrogen utilization by a plant is desirable. Expression of a glutamatc dehydrogenase gene in plants, e.g., E. coli gdhA genes, can lead to increased fixation of nitrogen in organic compounds. Furthermore, expression of gdhA in plants can lead to enhanced resistance to the herbicide glufosinate by incorporation of excess ammonia into glutamate, thereby detoxifying the ammonia. Expression of a novel gene can make a nutrient source available that was previously not accessible, e.g., an enzyme that releases a component of nutrient value from a more complex molecule.

Genes encoding polypeptides for improving nitrogen flow, sensing, uptake, storage and/or transport include those involved in aspartate, glutamine or glutamate biosynthesis, polypeptides involved in aspartate, glutamine or glutamate transport, polypeptides associated with the TOR (Target of Rapamycin) pathway, nitrate transporters, nitrate reductascs, amino transferases, ammonium transporters, chlorate transporters or polypeptides involved in tetrapyrrole biosynthesis are useful to include on MCs.

Genes encoding polypeptides for increasing the rate of photosynthesis include phytochrome, ribulose bisphosphate carboxylase-oxygenase, Rubisco activase, photosystem I and II proteins, electron carriers, ATP synthasc, NADH dehydrogenase or cytochrome oxidase are useful to deliver via MCs.

Genes encoding polypeptides for increasing phosphorus uptake, transport or utilization include phosphatases or phosphate transporters are also useful to include on MCs.

Male Sterility

Male sterility is useful in the production of hybrid seed. Male sterility can be produced through expression of exogenous genes. For example, expression of genes that encode proteins, RNAs, or peptides that interfere with development of the male inflorescence and/or gametophyte result in male sterility. Chimeric ribonuclease genes that express in the anthers of transgenic tobacco and oilseed rape lead to male sterility (Mariani, Beuckeleer et al. 1990).

A number of maize mutations confer cytoplasmic male sterility. One mutation, T cytoplasm, also correlates with sensitivity to Southern corn leaf blight. A DNA sequence, designated TURF 13 (Levings 1990), correlates with T cytoplasm. By delivery TURF 13 on a MC, to separate male sterility from disease sensitivity. As it is necessary to be able to restore male fertility for breeding purposes and for grain production, it is proposed that genes encoding restoration of male fertility also can be introduced. Similar cytoplasmic male sterility systems are know in cotton (Liu, Guo et al. 2003).

Altered Nutritional Content

Genes can be introduced into plants to improve or alter the nutrient quality or content of a particular crop. Introduction of genes that alter the nutrient composition of a crop can greatly enhance the feed or food value. For example, the protein of many crops is suboptimal for feed and food purposes, especially when fed to pigs, poultry, and humans. The protein is deficient in several amino acids that are essential in the diet of these species, requiring the addition of supplements to the grain. Limiting essential amino acids can include lysine, methionine, tryptophan, threonine, valine, arginine, and histidine. Some amino acids become limiting only after the crop is supplemented with other inputs for feed formulations. The levels of these essential amino acids in seeds and grain can be elevated by mechanisms that include the introduction of genes to increase the biosynthesis of the amino acids, decrease the degradation of the amino acids, increase the storage of the amino acids in proteins, or increase transport of the amino acids to the seeds or grain.

Polypeptides useful for providing increased seed protein quantity and/or quality include those involved in the metabolism of amino acids in plants, particularly polypeptides involved in biosynthesis of methionine/cysteine and lysine, amino acid transporters, amino acid efflux carriers, seed storage proteins, proteases, or polypeptides involved in phytic acid metabolism.

The protein composition of a crop can be altered to improve the balance of amino acids in a variety of ways including elevating expression of native proteins, decreasing expression of those with poor composition, changing the composition of native proteins, or introducing genes encoding entirely new proteins possessing superior composition.

The introduction of genes that alter the oil content of a crop plant can also be of value. Increases in oil content can result in increases in metabolizable-energy-content and density of the seeds for use in feed and food. The introduced genes can encode enzymes that remove or reduce rate-limitations or regulated steps in fatty acid or lipid biosynthesis. Such genes can include those that encode acetyl-CoA carboxylase, ACP-acyltransferase, alpha-ketoacyl-ACP synthase, or other well known fatty acid biosynthetic activities. Other possibilities are genes that encode proteins that do not possess enzymatic activity such as acyl carrier protein. Genes can be introduced that alter the balance of fatty acids present in the oil providing a more healthful or nutritive feedstuff. The introduced DNA also can encode sequences that block expression of enzymes involved in fatty acid biosynthesis, altering the proportions of fatty acids present in crops.

Genes can be introduced that enhance the nutritive value of crops, or of foods derived from crops by increasing the level of naturally occurring phytosterols, or by encoding for proteins to enable the synthesis of phytosterols in crops. The phytosterols from these crops can be processed directly into foods, or extracted and used to manufacture food products.

Genes can be introduced that enhance the nutritive value of the starch component of crops, for example by increasing the degree of branching, resulting in improved utilization of the starch in livestock by delaying its metabolism. Additionally, other major constituents of a crop can be altered, including genes that affect a variety of other nutritive, processing, or other quality aspects. For example, pigmentation can be increased or decreased.

Carbohydrate metabolism can be altered, for example by increased sucrose production and/or transport. Polypeptides useful for affecting carbohydrate metabolism include polypeptides involved in sucrose or starch metabolism, carbon assimilation or carbohydrate transport, including, for example sucrose transporters or glucose/hexose transporters, enzymes involved in glycolysis/gluconeogenesis, the pentose phosphate cycle, or raffinose biosynthesis, or polypeptides involved in glucose signaling, such as SNF1 complex proteins.

Feed or food crops can also possess sub-optimal quantities of vitamins, antioxidants or other nutraceuticals, requiring supplementation to provide adequate nutritive value and ideal health value. Introduction of genes that enhance vitamin biosynthesis can be envisioned including, for example, vitamins A, E, B12, choline, or the like. Mineral content can also be sub-optimal. Thus genes that affect the accumulation or availability of compounds containing phosphorus, sulfur, calcium, manganese, zinc, or iron among others would be valuable.

Numerous other examples of improvements of crops can be used with the invention. The improvements do not necessarily involve fiber, but can, for example, improve the value of a crop for silage. Introduction of DNA to accomplish this include sequences that alter lignin production such as those that result in the “brown midrib” phenotype associated with superior feed value for cattle. Other genes can encode for enzymes that alter the structure of extracellular carbohydrates in the stover, or that facilitate the degradation of the carbohydrates in the silage portion of the crop so that it can be efficiently fermented into ethanol or other useful carbohydrates.

Modifying the nutritional content of plants can also be useful, such as reducing undesirable components, including fats, starches, etc. This can be done, for example, by the use of exogenous nucleic acids that encode enzymes that increase plant use or metabolism of such components so that they are present at lower quantities. Alternatively, it can be done by use of exogenous nucleic acids that reduce expression levels or activity of native plant enzymes that synthesize such components.

Likewise the elimination of certain undesirable traits can improve the food or feed value of the crop. Many undesirable traits must currently be eliminated by special post-harvest processing steps and the degree to that these can be engineered into the plant prior lo harvest and processing would provide significant value. Examples of such traits are the elimination of anti-nutritionals such as phytates, gossypol and other phenolic compounds that are commonly found in many crop species. Also, the reduction of fats, carbohydrates and certain phyto-hormones can be valuable for the food and feed industries as they can allow a more efficient mechanism to meet specific dietary requirements.

In addition to direct improvements in feed or food value, genes also can be introduced that improve the processing of crops and improve the value of the products resulting from the processing. Processing steps can include mechanical harvesting, stripping, ginning, milling and wet-milling. Thus novel genes that increase the efficiency and reduce the cost of such processing can also find use. Improving the value of processing products can include altering the quantity or quality of starch, oil, gluten meal, or the components of gluten feed. Elevation of starch can be achieved through the identification and elimination of rate limiting steps in starch biosynthesis by expressing increased amounts of enzymes involved in biosynthesis or by decreasing levels of the other components of crops resulting in proportional increases in starch.

Oil, such as cottonseed oil, can be improved by introduction and expression of genes. Oil properties can be altered to improve its performance in the production and use of cooking oil, shortenings, lubricants or other oil-derived products or improvement of its health attributes when used in the food-related applications. Novel fatty acids also can be synthesized that upon extraction can serve as starting materials for chemical syntheses. The changes in oil properties can be achieved by altering the type, level, or lipid arrangement of the fatty acids present in the oil. This in turn can be accomplished by the addition of genes that encode enzymes that catalyze the synthesis of novel fatty acids (e.g., fatty acid clongascs, desaturases) and the lipids possessing them or by increasing levels of native fatty acids while reducing levels of precursors or breakdown products. Alternatively, DNA sequences can be introduced that slow or block steps in fatty acid biosynthesis resulting in the increase in precursor fatty acid intermediates. Genes that might be added include desaturases, epoxidases, hydratases, dehydratases, or other enzymes that catalyze reactions involving fatty acid intermediates. Representative examples of catalytic steps that might be blocked include the desaturations from stearic to oleic acid or oleic to linolenic acid resulting in the respective accumulations of stearic and oleic acids. Another example is the blockage of elongation steps resulting in the accumulation of C8 to C12 saturated fatty acids.

Genes encoding polypeptides useful for providing increased seed oil quantity and/or quality include polypeptides involved in fatty acid and glycerolipid biosynthesis, beta-oxidation enzymes, enzymes involved in biosynthesis of nutritional compounds, such as carotenoids and tocopherols, or polypeptides that increase embryo size or number or thickness of aleurone are useful to include on MCs.

Genes encoding polypeptides involved in production of galactomannans or arabinogalactans are of interest for providing plants that have increased and/or modified reserve polysaccharides for use in food, pharmaceutical, cosmetic, paper and paint industries can also be delivered on MCs.

Genes encoding polypeptides involved in modification of flavonoid/isoflavonoid metabolism in plants include cinnamate-4-hydroxylase, chalcone synthase or flavones synthase are also useful to include on MCs. Enhanced or reduced activity of such polypeptides in modified plants will provide changes in the quantity and/or speed of flavonoid metabolism in plants and can improve disease resistance by enhancing synthesis of protective secondary metabolites or improving signaling pathways governing disease resistance.

Genes encoding polypeptides involved in lignin biosynthesis are of interest for increasing plants' resistance to lodging and for increasing the usefulness of plant materials as biofuels can be delivered on MCs.

Production or Assimilation of Chemicals or Biological Compounds

Modified plants can be made that can be used for the production or manufacturing of useful biological compounds that were either not produced at all, or not produced at the same level, in the plant previously. Alternatively, plants produced according to the invention can be engineered to metabolize or absorb and concentrate certain compounds, such as hazardous wastes, thereby allowing bioremediation of these compounds.

The vast array of possibilities include any biological compound that is presently produced by any organism, such as proteins, nucleic acids, primary and intermediary metabolites, carbohydrate polymers, enzymes for uses in bioremediation, enzymes for modifying pathways that produce secondary plant metabolites such as falconoid or vitamins, enzymes that could produce pharmaceuticals, and for introducing enzymes that could produce compounds of interest to the manufacturing industry such as specialty chemicals and plastics. The compounds can be produced by the plant, extracted upon harvest and/or processing, and used for any presently recognized useful purpose such as pharmaceuticals, fragrances, and industrial enzymes.

Other Characteristics

Cell cycle modification: Polypeptides encoding cell cycle enzymes and regulators of the cell cycle pathway are useful for manipulating growth rate in plants to provide early vigor and accelerated maturation. Improvements in quality traits, such as seed oil content, can also be obtained by expression of cell cycle enzymes and cell cycle regulators. Polypeptides of interest for modification of cell cycle pathway include cyclin and EF5 alpha pathway proteins, polypeptides involved in polyamine metabolism, polypeptides that act as regulators of the cell cycle pathway, including cyclin-dependent kinases (CDKs), CDK-activating kinases, cell cycle-dependent phosphatases, CDIC-inhibitors, Rb and Rb-binding proteins, or transcription factors that activate genes involved in cell proliferation and division, such as the E2F family of transcription factors, proteins involved in degradation of cyclins and plant homologs of tumor suppressor polypeptides.

Plant growth regulators: Polypeptides involved in production of substances that regulate the growth of various plant tissues are of interest in the present invention and can be used to provide modified plants having altered morphologies and improved plant growth and development profiles leading to improvements in yield and stress response. Of particular interest are polypeptides involved in the biosynthesis, or degradation of plant growth hormones, such as gibberellins, brassinosteroids, cytokinins, auxins, ethylene or abscisic acid, and other proteins involved in the activity, uptake and/or transport of such polypeptides, including for example, cytokinin oxidase, cytokinin/purine permeases, F-box proteins, G-proteins or phytosulfokines.

Transcription factors in plants: Transcription factors play a key role in plant growth and development by controlling the expression of one or more genes in temporal, spatial and physiological specific patterns. Enhanced or reduced activity of such polypeptides in modified plants can provide significant changes in gene transcription patterns and provide a variety of beneficial effects in plant growth, development and response to environmental conditions. Transcription factors of interest include myb transcription factors, including hefix-turn-helix proteins, homeodomain transcription factors, leucine zipper transcription factors, MADS transcription factors, transcription factors having AP2 domains, zinc finger transcription factors, CCAAT binding transcription factors, ethylene responsive transcription factors, transcription initiation factors or UV damaged DNA binding proteins.

Homologous recombination: Increasing the rate of homologous recombination in plants is useful for accelerating the introgression of transgenes into breeding varieties by backcrossing, and to enhance the conventional breeding process by allowing rare recombinants between closely linked genes in phase repulsion to be identified more easily. Polypeptides useful for expression in plants to provide increased homologous recombination include polypeptides involved in mitosis and/or meiosis, DNA replication, nucleic acid metabolism, DNA repair pathways or homologous recombination pathways including for example, recombinases, nucleases, proteins binding to DNA double-strand breaks, single-strand DNA binding proteins, strand-exchange proteins, resolvases, ligases, helicases and polypeptide members of the RAD52 epistasis group.

Non-Protein-Expressing Exogenous Nucleic Acids

Plants with decreased expression of a gene of interest can also be achieved, for example, by expression of antisense nucleic acids, dsRNAmiRNAs, siRNAs, or RNAi, catalytic RNA such as ribozymes, sense expression constructs that exhibit cosuppression effects, or aptamers.

Antisense RNA reduces production of the polypeptide product of the target messenger RNA, for example by blocking translation through formation of RNA:RNA duplexes or by inducing degradation of the target mRNA. Antisense approaches are a way of preventing or reducing gene function by targeting the genetic material as disclosed in U.S. Pat. Nos. 4,801,540; 5,107,065; 5,759,829; 5,910,444; 6,184,439; and 6,198,026. In one approach, an antisense gene sequence is introduced that is transcribed into antisense RNA that is complementary to the target mRNA. For example, part or all of the normal gene sequences are placed under a promoter in inverted orientation so that the complementary strand is transcribed into a non-protein expressing antisense RNA. The promoter used for the antisense gene can influence the level, timing, tissue, specificity, or inducibility of the antisense inhibition.

Autonomous MCs can contain exogenous DNA bounded by recombination sites, for example lox-P sites, that can be recognized by a recombinase, e.g., Cre, and removed from the MC. In cases where there is a homologous recombination site or sites in the host genomic DNA, the exogenous DNA excised the MC can be integrated into the genome at one of the specific recombination sites and the DNA bounded by the recombination sites will become integrated into the host DNA. The use of a MC as a platform for DNA excision or for launching such DNA integration into the host genome can include in vivo induction of the expression of a recombinase encoded in the genomic DNA of a transgenic host, or in a MC or other episome.

RNAi gene suppression in plants by transcription of a dsRNA is described in U.S. Pat. No. 6,506,559, U.S. patent application Publication No. 2002/0168707, WO 98/53083, WO 99/53050 and WO 99/61631. The double-stranded RNA or RNAi constructs can trigger the sequence-specific degradation of the target messenger RNA. Suppression of a gene by RNAi can be achieved using a recombinant DNA construct having a promoter operably linked to a DNA element comprising a sense and anti-sense element of a segment of genomic DNA of the gene, e.g., a segment of at least about 23 nucleotides, more preferably about 50 to 200 nucleotides where the sense and anti-sense DNA components can be directly linked or joined by an intron or artificial DNA segment that can form a loop when the transcribed RNA hybridizes to form a hairpin structure.

miRNA (microRNAs) are short RNA sequences, approximately 22 nucleotides long, that are capable of binding to complementary sequences in the 3′ UTR of target mRNAs. Binding of the miRNA to the target mRNAs can result in their silencing. Genes encoding artificial miRNAS (amiRNAs) can be engineered to express amiRNAs that silence specific mRNAs (Schwab, Ossowski et al. 2006). Genes encoding miRNAs or amiRNAs can be included on MCs.

Small interfering RNA (siRNA) are short double-stranded RNA molecules (20-25 nucleotides long) that can mediate the RNAi pathway and silence the expression of specific genes. These RNAs are also thought to play a role in protection from viruses, and modulating chromatin structure. Like miRNA, it is also possible to engineer genes to encode artificial siRNA for the purpose of gene silencing (de la Luz Gutierrez-Nava, Aukerman et al. 2008). Genes encoding siRNAs can be included on MCs.

Catalytic RNA molecules or ribozymes can also be used to inhibit expression of the target gene or genes or facilitate molecular reactions. Ribozymes are targeted to a given sequence by hybridization of sequences within the ribozyme to the target mRNA. Two stretches of homology are required for this targeting, and these stretches of homologous sequences flank the catalytic ribozyme structure. It is possible to design ribozymes that specifically pair with virtually any target mRNA and cleave the target mRNA at a specific location, thereby inactivating it. A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include Tobacco Ringspot Virus, Avocado Sunblotch Viroid, and Lucerne Transient Streak Virus, and the satellite RNAs from velvet tobacco mottle virus, Solanum nodiflorum mottle virus and subterranean clover mottle virus. The design and use of target RNA-specific ribozymes has been described (Haseloff and Gerlach 1988). Several different ribozyme motifs have been described with RNA cleavage activity (Symons 1992). Other suitable ribozymes include sequences from RNase P with RNA cleavage activity (U.S. Pat. Nos. 5,168,053 and 5,624,824), hairpin ribozyme structures (Chowrira, Pavco et al. 1994) and Hepatitis Delta virus based ribozymes (U.S. Pat. No. 5,625,047).

Another method of reducing protein expression utilizes the phenomenon of co-suppression or gene silencing (for example, U.S. Pat. No. 6,063,947; 5,686,649; or 5,283,184). Co-suppression of an endogenous gene can be accomplished using a full-length cDNA sequence as well as a partial cDNA sequence. The phenomenon of co-suppression has also been used to inhibit plant target genes in a tissue-specific manner.

In some embodiments, nucleic acids from one species of plant are expressed in another species of plant to effect co-suppression of a homologous gene. The introduced sequence generally will be substantially identical to the endogenous sequence intended to be repressed, for example, about 65%, 80%, 85%, 90%, or preferably 95% or greater identical. Higher identity can result in a more effective repression of expression of the endogenous sequence. A higher identity in a shorter than full length sequence compensates for a longer, less identical sequence. Furthermore, the introduced sequence need not have the same intron or exon pattern, and identity of non-coding segments will be equally effective. Generally, where inhibition of expression is desired, some transcription of the introduced sequence occurs. The effect can occur where the introduced sequence contains no coding sequence per se, but only intron or untranslated sequences homologous to sequences present in the primary transcript of the endogenous sequence.

Yet another method of reducing protein activity is by expressing nucleic acid ligands, aptamers, that specifically bind to the protein. Aptamers can be obtained by the SELEX (Systematic Evolution of Ligands by Exponential Enrichment) method (U.S. Pat. No. 5,270,163).

Other examples of non-protein expressing sequences specifically envisioned for use with the invention include tRNA sequences, for example, to alter codon usage, and rRNA variants, for example, that can confer resistance to various agents such as antibiotics.

Unexpressed DNA sequences, including novel synthetic sequences, can be introduced into cells as proprietary “labels” of those cells and plants and seeds thereof. It would not be necessary for a label DNA element to disrupt the function of a gene endogenous to the host organism, as the sole function of this DNA would be to identify the origin of the organism. For example, one could introduce a unique DNA sequence into a plant and this DNA element would identify all cells, plants, and progeny of these cells as having arisen from that labeled source. It is proposed that inclusion of label DNAs would enable one to distinguish proprietary germplasm or germplasm derived from such, from unlabelled germplasm.

### E. Exemplary Plant Promoters, Regulatory Sequences and Targeting Sequences

Exemplary classes of plant promoters are described below.

Constitutive Expression promoters: Exemplary constitutive expression promoters include the ubiquitin promoter, the CaMV 35S promoter (U.S. Pat. Nos. 5,858,742 and 5,322,938); and the actin promoter (e.g., rice—U.S. Pat. No. 5,641,876).

Inducible Expression promoters: Exemplary inducible expression promoters include the chemically regulatable tobacco PR-1 promoter (e.g., tobacco—U.S. Pat. No. 5,614,395; maize—U.S. Pat. No. 6,429,362). Various chemical regulators can be used to induce expression, including the benzothiadiazole, isonicotinic acid, and salicylic acid compounds disclosed in U.S. Pat. Nos. 5,523,311 and 5,614,395. Other promoters inducible by certain alcohols or ketones, such as ethanol, include the alcA gene promoter from Aspergillus nidulans (Caddick et al., 1998). Glucocorticoid-mediated induction systems can also be used (Aoyama and Chua 1997). Another class of useful promoters are water-deficit-inducible promoters, e.g., promoters that are derived from the 5′ regulatory region of genes identified as a heat shock protein 17.5 gene (HSP 17.5), an HVA22 gene (HVA22), and a cinnamic acid 4-hydroxylasc gene (CA4H) of Zea mays. Another water-deficit-inducible promoter is derived from the rab-17 promoter. U.S. Pat. No. 6,084,089 discloses cold inducible promoters, U.S. Pat. No. 6,294,714 discloses light inducible promoters, U.S. Pat. No. 6,140,078 discloses salt inducible promoters, U.S. Pat. No. 6,252,138 discloses pathogen inducible promoters, and U.S. Pat. No. 6,175,060 discloses phosphorus deficiency inducible promoters.

Wound-inducible promoters can also be used, e.g., (Mialhe and Miller, 1994; Warner et al., 1993; Xu et al., 1993).

Tissue-Specific Promoters: Exemplary promoters that express genes only in certain tissues are useful. For example, root-specific expression can be attained using the promoter of the maize metallothionein-like (MTL) gene (U.S. Pat. No. 5,466,785). U.S. Pat. No. 5,837,848 discloses a root-specific promoter. Another exemplary promoter confers pith-preferred expression (maize trpA gene and promoter; WO 93/07278). Leaf-specific expression can be attained, for example, by using the promoter for a maize gene encoding phosphoenol carboxylase. Pollen-specific expression can be conferred by the promoter for the maize calcium-dependent protein kinase (CDPK) gene that is expressed in pollen cells (WO 93/07278). U.S. Pat. Appl. Pub. No. 20040016025 describes tissue-specific promoters. Pollen-specific expression can also be conferred by the tomato LAT52 pollen-specific promoter. U.S. Pat. No. 6,437,217 discloses a root-specific maize RS81 promoter, U.S. Pat. No. 6,426,446 discloses a root specific maize RS324 promoter, U.S. Pat. No. 6,232,526 discloses a constitutive maize A3 promoter, U.S. Pat. No. 6,177,611 that discloses constitutive maize promoters, U.S. Pat. No. 6,433,252 discloses a maize L3 oleosin promoter that are aleurone and seed coat-specific promoters, U.S. Pat. No. 6,429,357 discloses a constitutive rice actin 2 promoter and intron, U.S. patent application Pub. No. 20040216189 discloses an inducible constitutive leaf-specific maize chloroplast aldolase promoter.

Optionally a plant transcriptional terminator can be used in place of the plant-expressed gene native transcriptional terminator. Exemplary transcriptional terminators are those that are known to function in plants and include the CaMV 35S terminator, the tml terminator, the nopaline synthase terminator and the pea rbcS E9 terminator. These can be used in both monocotyledons and dicotyledons.

Various intron sequences have been shown to enhance expression. For example, the introns of the maize Adh1 gene can significantly enhance expression, especially intron 1 (Callis, Fromm et al. 1987). The intron from the maize bronze) gene also enhances expression. Intron sequences have been routinely incorporated into plant transformation vectors, typically within the non-translated leader. U.S. Patent Application Publication 2002/0192813 discloses 5′, 3′ and intron elements useful in the design of effective plant expression vectors.

A number of non-translated leader sequences derived from viruses are also known to enhance expression, and these are particularly effective in dicotyledonous cells. Specifically, leader sequences from Tobacco Mosaic Virus (TMV, the “omega-sequence”), Maize Chlorotic Mottle Virus (MCMV), and Alfalfa Mosaic Virus (AMV) can enhance expression. Other leader sequences known and include: picornavirus leaders, for example, EMCV leader (Encephalomyocarditis 5′ noncoding region); potyvirus leaders, for example, TEV leader (Tobacco Etch Virus); MDMV leader (Maize Dwarf Mosaic Virus); human immunoglobulin heavy-chain binding protein (BiP) leader; untranslated leader from the coat protein mRNA of alfalfa mosaic virus (AMV RNA 4); tobacco mosaic virus leader (TMV); or Maize Chlorotic Mottle Virus leader (MCMV).

A minimal promoter can also be incorporated. Such a promoter has low background activity in plants when there is no transactivator present or when enhancer or response element binding sites are absent. An example is the BzI minimal promoter, obtained from the bronze) gene of maize. A minimal promoter can also be created by use of a synthetic TATA element. The TATA element allows recognition of the promoter by RNA polymerase factors and confers a basal level of gene expression in the absence of activation.

Sequences controlling the targeting of gene products also can be included. For example, the targeting of gene products to the chloroplast is controlled by a signal sequence found at the amino terminal end of various proteins that is cleaved during chloroplast import to yield the mature protein (e.g., (Comai et al., 1988)). These signal sequences can be fused to heterologous gene products to import heterologous products into the chloroplast. DNA encoding for appropriate signal sequences can be isolated from the 5′ end of the cDNAs encoding the RUBISCO protein, the CAB protein, the EPSP synthasc enzyme, the GS2 protein or many other proteins that are known to be chloroplast localized. Other gene products are localized to other organelles, such as the mitochondrion and the peroxisome (e.g., (Unger, Hand et al. 1989)). Examples of sequences that target to such organelles are the nuclear-encoded ATPases or specific aspartate amino transferase isoforms for mitochondria. Amino terminal and carboxy-terminal sequences are responsible for targeting to the ER, the apoplast, and extracellular secretion from aleurone cells. Amino terminal sequences in conjunction with carboxy terminal sequences can target to the vacuole.

Another element that can be introduced is a matrix attachment region element (MAR), such as the chicken lysozyme A element that can be positioned around an expressible gene of interest to effect an increase in overall expression of the gene and diminish position dependent effects upon incorporation into the plant genome.

Use of Non-Plant Promoter Regions Isolated from Drosophila melanogaster and Saccharomyces cerevisiae to Express Genes in Plants

The promoter in the MC of the present invention can be derived from plant or non-plant species. For example, the nucleotide sequence of the promoter is derived from non-plant species for the expression of genes in plant cells, such as dicotyledon plant cells, such as cotton. Non-plant promoters can be constitutive or inducible promoters derived from insects, e.g., Drosophila melanogaster, or from yeast, e.g., Saccharomyces cerevisiae. Table 2 lists some of the promoters from Drosophila melanogaster and Saccharomyces cerevisiae that can be used as non-plant promoters; this list is not exhaustive. Promoters derived from any animal, protist, or fungi can also be used. SEQ ID NOS:1-20 are examples of promoter sequences derived from Drosophila melanogaster or Saccharomyces cerevisiae. These non-plant promoters can be operably linked to nucleic acid sequences encoding polypeptides or non-protein-expressing sequences including antisense RNA, miRNA, siRNA, and ribozymes, to form nucleic acid constructs, vectors, and host cells (prokaryotic or eukaryotic), comprising the promoters.

TABLE 2
Examples of non-plant promoters from Drosophila melanogaster
and Sacchromyces cerevisiae

| Drosophila melanogaster | | | Sacchromyces cerevisiae | | |
|---|---|---|---|---|---|
| SEQ | | Promoter gene | SEQ | | |
| ID NO: | Symbol | name | ID NO: | Symbol | Promoter gene |
| 1 | gd | Phosphogluconate | 7 | ef-2 | TEF2 |
| | | dehydrogenase | 8 | eu-1 | LEU1 |
| 2 | rim | grim | 9 | Et16 | METhionine |
| 3 | ro | Urate oxidase | | | requiring |
| 4 | na | snail | 10 | eu-2 | LEU2 |
| 5 | h3 | Rhodopsin 3 | 11 | is-4 | HIS4 |
| 6 | sp-1γ | Larval serum | 12 | et-2 | MET2 |
| | | protein 1 γ | 13 | tc-3 | STE3 |
| | | | 14 | rg-1 | ARG1 |
| | | | 15 | gk-1 | PGK1 |
| | | | 16 | PD-1 | GPD1 |
| | | | 17 | DH1 | ADH1 |
| | | | 18 | PD-2 | GPD2 |
| | | | 19 | rg-4 | ARGinine |
| | | | | | requiring |
| | | | 20 | at-1 | YAT-1 |

The present invention relates to methods for producing a polypeptide, comprising cultivating plant material for the production of the polypeptide at any level, wherein the plant host cells comprises a first nucleic acid sequence encoding the polypeptide operably linked to a second nucleic acid sequence comprising a heterologous promoter foreign to the nucleic acid sequence, wherein the promoter comprises a sequence selected from the group consisting of SEQ ID NOS:1-20 or subsequences thereof; and mutant, hybrid, or tandem promoters thereof that retain promoter activity.

The present invention also relates to methods for producing non-protein expressed sequences, comprising cultivating plant material for the production of the non-protein expressed sequence, wherein the plant host cell comprises a first nucleic acid sequence encoding the non-protein expressed sequences operably linked to a second nucleic acid sequence comprising a heterologous promoter foreign to the nucleic acid sequence, wherein the promoter comprises a sequence selected from the group consisting of SEQ ID NOS: 1 to 20 or subsequences thereof; and mutant, hybrid, or tandem promoters thereof.

The present invention also relates to isolated promoter sequences and to constructs, vectors, or plant host cells comprising one or more of the promoters operably linked to a nucleic acid sequence encoding a polypeptide or non-protein expressing sequence.

In the methods of the present invention, the promoter can also be a mutant of the promoters having a substitution, deletion, and/or insertion of one or more nucleotides in the nucleic acid sequence of SEQ ID NOS:1-20.

The present invention also relates to methods for obtaining derivative promoters of SEQ ID NOS:1-20.

The techniques used to isolate or clone a nucleic acid sequence comprising a promoter of interest are known in the art and include isolation from genomic DNA. The cloning procedures can involve excision or amplification, for example by polymerase chain reaction (PCR), and isolation of a desired nucleic acid fragment comprising the nucleic acid sequence encoding the promoter, insertion of the fragment into a vector molecule, and incorporation of the recombinant vector into the plant cell.

### F. Constructing MCs by Site-Specific Recombination

Plant MCs can be constructed using site-specific recombination sequences (for example those recognized by the bacteriophage P1 Cre recombinase, or the bacteriophage lambda integrase, or similar recombination enzymes). A compatible recombination site, or a pair of such sites, is present on both the centromere containing DNA clones and the donor DNA clones. Incubation of the donor clone and the centromere clone in the presence of the recombinase enzyme causes strand exchange to occur between the recombination sites in the two plasmids; the resulting MCs contain centromere sequences as well as MC vector sequences. The DNA molecules formed in such recombination reactions is introduced into E. coli, other bacteria, yeast or plant cells by common methods in the field including, heat shock, chemical transformation, electroporation, particle bombardment, whiskers, or other transformation methods followed by selection for marker genes, including chemical, enzymatic, or color markers present on either parental plasmid, allowing for the selection of transformants harboring MCs.

### G. Methods of Detecting and Characterizing MCs in Plant Cells or of Scoring MC Performance in Plant Cells

Identification of Candidate Centromere Fragments by Probing BAC Libraries

The invention also provides for a novel method for identifying centromere sequences that are neither highly methylated nor comprising of tandem repeats. In this method, all available genomic nucleic acid sequences from an organism are assembled into low-stringency contigs. Those contigs having the largest assemblies (i.e., many sequences aligned, “deep read”) are then further examined. The pool of “largest” assemblies can be the top 1%, 2%, 3%, 4%, 5%, 6%, 7%, or 10% or more. This pool of contigs is then examined first for contigs containing tandem repeats using commonly available software. These contigs are eliminated from the pool. A consensus sequence determined for the remaining contigs with the deepest reads. Probes are designed and synthesized based on the consensus squence, and used in an assay that allows for the detection of centromere sequences, such as fluorescence in situ hybridization (FISH) of mitotic or meiotic metaphase chromosomes. Of course, any suitable assay can be used. When using FISH, for example, a good candidate for a centromere sequence is a probe that labels every primary constriction of every chromosome (though genomes of allopolyploids may contain distinct sub-genomes with distinct centromeres). If desired, the candidate sequence can be further tested with other morphological or functional assays.

Methods for determining consensus sequence are well known in the art, e.g., U.S. Pat. App. Pub. No. 20030124561; (Hall, Fiebig et al. 2002). These methods, including DNA sequencing, assembly, and analysis, are well known and there are many possible variations known to those skilled in the art. Other alignment parameters can also be useful such as using more or less stringent definitions of consensus.

Non-Selective MC Mitotic Inheritance Assays

The following assays can distinguish autonomous events from integrated events.

Assay #1: Transient Assay

MCs are tested for their ability to become established as chromosomes and their ability to be inherited in mitotic cell divisions. MCs are delivered to plant cells. The cells used can be at various stages of growth. In this example, a population in that some cells were undergoing division can be used. The MC is then assessed over the course of several cell divisions, by tracking the presence of a screenable marker, e.g., a visible marker gene such as one encoding a fluorescent protein. Following initial delivery into many single cells and several cell divisions, single transformed cells divide to form clusters of MC-containing cells if the MC is inherited well. Other exemplary embodiments of this method include delivering MCs to other mitotic cell types, including roots and shoot meristems.

Assay #2: Non-Lineage Based Inheritance Assays on Modified Transformed Cells and Plants

MC inheritance is assessed on modified cell lines and plants by following the presence of the MC over the course of multiple cell divisions. An initial population of MC containing cells is assayed for the presence of the MC, by the presence of a marker gene, such as a gene encoding a fluorescent protein, a colored protein, a protein assayable by histochemical assay, or a gene affecting cell morphology. All nuclei are stained with a DNA-specific dye including but not limited to DAPI, Hoechst 33258, OliGreen, Giemsa YOYO, or TOTO, allowing a determination of the number of cells that do not contain the MC. After the initial determination of the percent of cells carrying the MC, the cells are allowed to divide over the course of several cell divisions. The number of cell divisions, n, is determined by an appropriate method, such as monitoring the change in total weight of cells, monitoring the change in volume of the cells, or directly counting cells in an aliquot of the culture. After a number of cell divisions, the population of cells is again assayed for the presence of the MC. The loss rate per generation is calculated by the equation (I):

Loss rate per generation=1−(F/1)^{1/n} (I)

The population of MC-containing cells can include suspension cells, callus, roots, leaves, meristems, flowers, or any other tissue of modified plants, or any other cell type containing a MC.

Assay #3: Lineage-Based Inheritance Assays on Modified Cells and Plants

MC inheritance is assessed on modified cell lines and plants by following the presence of the MC over the course of multiple cell divisions. In cell types that allow for tracking of cell lineage, such as root cell files, trichomes, and leaf stomata guard cells, MC loss per generation does not need to be determined statistically over a population, it can be discerned directly through successive cell divisions. In other manifestations of this method, cell lineage can be discerned from cell position, or methods including but not limited to the use of histological lineage tracing dyes, and the induction of genetic mosaics in dividing cells.

In one example, the two guard cells of the stomata are daughters of a single precursor cell. To assay MC inheritance in this cell type, the epidermis of the leaf of a plant containing a MC is examined for the presence of the MC by the presence of a marker gene, including one encoding a fluorescent protein, a colored protein, a protein assayable by histochemical assay, or a gene affecting cell morphology. The number of loss events in which one guard cell contains the MC (L) and the number of cell divisions in which both guard cells contain the MC (B) are counted. The loss rate per cell division is determined as L/(L+B). Other lineage-based cell types are assayed in similar fashion. Similar assays have been used in yeast.

Lineal MC inheritance can also be assessed by examining root files or clustered cells in callus over time. Changes in the percent of cells carrying the MC indicate the mitotic inheritance.

Assay #4: Inheritance Assays on Modified Cells and Plants in the Presence of Chromosome Loss Agents

Assays #1-3 can be done in the presence of chromosome loss agents (e.g., colchicine, colcemid, caffeine, etopocide, nocodazole, oryzalin, and trifluran). It is likely that autonomous MCs are more susceptible to loss induced by chromosome loss agents; therefore, autonomous MCs show a lower rate of inheritance in the presence of chromosome loss agents. These methods have been used to study chromosome loss in fruit flies and yeast.

### H. Transformation of Plant Cells and Plant Regeneration

Various methods can be used to deliver DNA into plant cells. These include biological methods, such as Agrobacterium, E. coli, and viruses; physical methods, such as biolistic particle bombardment, nanocopiea device, the Stein beam gun, silicon carbide whiskers and microinjection; electrical methods, such as electroporation; and chemical methods, such as the use of polyethylene glycol and other compounds that stimulate DNA uptake into cells (Dunwell 1999) and U.S. Pat. No. 5,464,765.

Agrobacterium-Mediated Delivery

Several Agrobacterium species mediate the transfer of “T-DNA” that can be genetically engineered to carry a desired piece of DNA into many plant species. Plasmids used for delivery contain the T-DNA flanking the nucleic acid to be inserted into the plant. The major events marking the process of T-DNA mediated pathogenesis are induction of virulence genes, processing and transfer of T-DNA.

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be modified by Agrobacterium and (b) that the modified cells or tissues can be induced to regenerate into whole plants. The third method is transformation of seeds, apices or meristems with Agrobacterium. This method requires exposure of the meristematic cells of these tissues to Agrobacterium and micropropagation of the shoots or plant organs arising from these meristematic cells.

Those of skill in the art are familiar with procedures for growth and suitable culture conditions for Agrobacterium, as well as subsequent inoculation procedures. Liquid or semi-solid culture media can be used. The density of the Agrobacterium culture used for inoculation and the ratio of Agrobacterium cells to explant can vary from one system to the next, as can media, growth procedures, timing and lighting conditions.

Transformation of dicotyledons using Agrobacterium has long been known in the art, and transformation of monocotyledons using Agrobacterium has also been described (WO 94/00977; U.S. Pat. No. 5,591,616; US20040244075).

A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes that cause tumorigenesis or rhizogenesis. Exemplary strains include Agrobaclerium tumefaciens strain CSS, a nopaline-type strain that is used to mediate the transfer of DNA into a plant cell, octopine-type strains such as LBA4404 or succinamopine-type strains, e.g., EHA101 or EHA105.

The efficiency of transformation by Agrobacterium can be enhanced by using a number of methods known in the art. For example, the inclusion of a natural wound response molecule such as acetosyringone (AS) to the Agrobaclerium culture can enhance transformation efficiency with Agrobaclerium tumefaciens. Alternatively, transformation efficiency can be enhanced by wounding the target tissue to be modified or transformed. Wounding of plant tissue can be achieved, for example, by punching, maceration, bombardment with microprojectiles, etc.

In addition, transfer of a disarmed Ti plasmid without T-DNA and another vector with T-DNA containing the marker enzyme beta-glucuronidase can be accomplished into three different bacteria other than Agrobacteria (Broothaerts et al., 2005), which adds to the transformation vector arsenal.

Micro Projectile Bombardment Delivery

In this process, the desired nucleic acid is deposited on or in small dense particles, e.g., tungsten, platinum, or preferably 1 micron gold particles, that are then delivered at a high velocity into the plant tissue or plant cells using a specialized biolistics device, such as are available from Bio-Rad Laboratories (Hercules, Calif.). The advantage of this method is that no specialized sequences need to be present on the nucleic acid molecule to be delivered into plant cells.

For bombardment, cells in suspension are concentrated on filters or solid culture medium. Alternatively, immature embryos, seedling explants, or any plant tissue or target cells can be arranged on solid culture medium. The cells to be bombarded are positioned at an appropriate distance below the microprojectile stopping plate.

Various biolistics protocols have been described that differ in the type of particle or the manner in that DNA is coated onto the particle. Any technique for coating microprojectiles that allows for delivery of transforming DNA to the target cells can be used. For example, particles can be prepared by functionalizing the surface of a gold oxide particle by providing free amine groups. DNA, having a strong negative charge, binds to the functionalized particles.

Parameters such as the concentration of DNA used to coat microprojectiles can influence the recovery of transformants containing a single copy of the transgene. For example, a lower concentration of DNA may not necessarily change the efficiency of the transformation but can instead increase the proportion of single copy insertion events. Ranges of approximately 1 ng to approximately 10 pg, approximately 5 ng to 8 μg or approximately 20 ng, 50 ng, 100 ng, 200 ng, 500 ng, 1 pg, 2 μg, 5 μg, or 7 μg of transforming DNA can be used per each 1.0-2.0 mg of starting 1.0 micron gold particles.

Other physical and biological parameters can be varied, such as manipulation of the DNA/microprojectile precipitate, factors that affect the flight and velocity of the projectiles, manipulation of the cells before and immediately after bombardment (including osmotic state, tissue hydration and the subculture stage or cell cycle of the recipient cells), the orientation of an immature embryo or other target tissue relative to the particle trajectory, and also the nature of the transforming DNA, such as linearized DNA or intact supercoiled plasmids. Physical parameters such as DNA concentration, gap distance, flight distance, tissue distance, and helium pressure, can be optimized.

The particles delivered via biolistics can be “dry” or “wet.” In the “dry” method, the MC DNA-coated particles such as gold are applied onto a macrocarrier (such as a metal plate, or a carrier sheet made of a fragile material, such as mylar) and dried. The gas discharge then accelerates the macrocarrier into a stopping screen that halts the macrocarrier but allows the particles to pass through. The particles are accelerated at, and enter, the plant tissue arrayed below on growth media. The media surrports plant tissue growth and development and are suitable for plant transformation and regeneration. These tissue culture media can either be purchased as a commercial preparation, or custom prepared and modified. Examples of such media include Murashige and Skoog (MS), N6, Linsmaier and Skoog, Uchimiya and Murashige, Gamborg's B5 media, D medium, MCCown's Woody plant media, Nitsch and Nitsch, and Schenk and Hildebrandt. Those of skill in the art are aware that media and media supplements such as nutrients and growth regulators for use in transformation and regeneration and other culture conditions such as light intensity during incubation, pH, and incubation temperatures can be optimized.

Those of skill in the art can use, devise, and modify selective regimes, media, and growth conditions depending on the plant system and the selective agent. Typical selective agents include antibiotics, such as geneticin (G418), kanamycin, paromomycin; or other chemicals, such as glyphosate or other herbicides.

MC Delivery without Selection

The MC is delivered to plant cells or tissues, e.g., plant cells in suspension to obtain stably modified callus clones for inheritance assays. Suspension cells are maintained in a growth media, for example Murashige and Skoog (MS) liquid medium containing an auxin such as 2,4-dichlorophenoxyacetic acid (2,4-D). Cells are bombarded using a particle bombardment process and propagated in the same liquid medium to permit the growth of modified and unmodified cells. Portions of each bombardment are monitored for formation of fluorescent clusters, which are then isolated by micromanipulation and cultured on solid medium. Clones modified with the MC are expanded, and homogenous clones are used in inheritance assays, or assays measuring MC structure or autonomy.

MC Transformation with Selectable Marker Gene

MC-modified cells in bombarded calluses or explants can be isolated using a selectable marker gene. The bombarded tissues are transferred to a medium containing an appropriate selective agent. Tissues are transferred into selection between 0 and about 7 days or more after bombardment. Selection of MC-modified cells can be further monitored by tracking fluorescent marker genes or by the appearance of modified explants (modified cells on explants can be green under light in selection medium, while surrounding non-modified cells are weakly pigmented). In plants that develop through shoot organogenesis (e.g., Brassica, tomato or tobacco), the modified cells can form shoots directly, or alternatively, can be isolated and expanded for regeneration of multiple shoots transgenic for the MC. In plants that develop through embryogenesis (e.g., corn or soybean), additional culturing steps may be necessary to induce the modified cells to form an embryo and to regenerate in the appropriate media.

For selection to be effective, the plant cells or tissue need to be grown on selective medium containing the appropriate concentration of antibiotic or killing agent, and the cells need to be plated at a defined and constant density. The concentration of selective agent and cell density are generally chosen to cause complete growth inhibition of wild type plant tissue that does not express the selectable marker gene; but allowing cells containing the introduced DNA to grow and expand into adchromosomal clones. This critical concentration of selective agent typically is the lowest concentration at that there is complete growth inhibition of wild type cells, at the cell density used in the experiments. However, in some cases, sub-killing concentrations of the selective agent can be equally or more effective for the isolation of plant cells containing MC DNA, especially in cases where the identification of such cells is assisted by a visible marker gene (e.g., fluorescent protein gene) present on the MC.

In some species (e.g., tobacco or tomato), a homogenous clone of modified cells can also arise spontaneously when bombarded cells are placed under the appropriate selection. An exemplary selective agent is the neomycin phosphotransferase II (NptII) marker gene that confers resistance to the antibiotics kanamycin, G418 (geneticin) and paramomycin. In other species, or in certain plant tissues or when using particular selectable markers, homogeneous clones may not arise spontaneously under selection; in this case the clusters of modified cells can be manipulated to homogeneity using the visible marker genes present on the MCs as an indication of that cells contain MC DNA.

Regeneration of Adchromosomal Plants from Explants to Mature, Rooted Plants

For plants that develop through shoot organogenesis (e.g., Brassica, tomato and tobacco), regeneration of a whole plant involves culturing of regenerable explant tissues taken from sterile organogenic callus tissue, seedlings or mature plants on a shoot regeneration medium for shoot organogenesis, and rooting of the regenerated shoots in a rooting medium to obtain intact whole plants with a fully developed root system.

For plant species, such cotton, corn and soybean, regeneration of a whole plant occurs via an embryogenic step that is not necessary for plant species where shoot organogenesis is efficient. In these plants, the explant tissue is cultured on an appropriate media for embryogenesis, and the embryo is cultured until shoots form. The regenerated shoots are cultured in a rooting medium to obtain intact whole plants with a fully developed root system.

Explants are obtained from any tissues of a plant suitable for regeneration. Exemplary tissues include hypocotyls, internodes, roots, cotyledons, petioles, cotyledonary petioles, leaves and peduncles, prepared from sterile seedlings or mature plants.

Explants are wounded (for example with a scalpel or razor blade) and cultured on a shoot regeneration medium (SRM) containing Murashige and Skoog (MS) medium as well as a cytokinin, e.g., 6-benzylaminopurinc (BA), and an auxin, e.g., a-naphthaleneacetic acid (NAA), and an anti-ethylene agent, e.g., silver nitrate (AgNO₃). For example, 2 mg/L of BA, 0.05 mg/L of NAA, and 2 mg/L of AgNO₃ can be added to MS medium for shoot organogenesis. The most efficient shoot regeneration is obtained from longitudinal sections of internode explants.

Shoots regenerated via organogenesis are rooted in a MS medium containing low concentrations of an auxin such as NAA.

To regenerate a whole plant with a MC, explants are pre-incubated for 1 to 7 days (or longer) on the shoot regeneration medium prior to bombardment with MC (see below). Following bombardment, explants are incubated on the same shoot regeneration medium for a recovery period up to 7 days (or longer), followed by selection for transformed shoots or clusters on the same medium but with a selective agent appropriate for a particular selectable marker gene (see below).

Method of Co-Delivering Growth Inducing Genes to Facilitate Isolation of Ad Chromosomal Plant Cell Clones

Another method used in the generation of cell clones containing MCs involves the co-delivery of DNA containing genes that are capable of activating growth of plant cells, or that promote the formation of a specific organ, embryo or plant structure that is capable of self-sustaining growth. In one embodiment, the recipient cell receives simultaneously the MC, and a separate DNA molecule encoding one or more growth promoting, organogenesis-promoting, embryo genesis-promoting or regeneration-promoting genes. Following DNA delivery, expression of the plant growth regulator genes stimulates the plant cells to divide, or to initiate differentiation into a specific organ, embryo, or other cell types or tissues capable of regeneration. Multiple plant growth regulator genes can be combined on the same molecule, or co-bombarded on separate molecules. Use of these genes can also be combined with application of plant growth regulator molecules into the medium used to culture the plant cells, or of precursors to such molecules that are converted to functional plant growth regulators by the plant cell's biosynthetic machinery, or by the genes delivered into the plant cell.

The co-bombardment strategy of MCs with separate DNA molecules encoding plant growth regulators transiently supplies the plant growth regulator genes for several generations of plant cells following DNA delivery. During this time, the MC can be stabilized by virtue of its centromere, but the DNA molecules encoding plant growth regulator genes, or organogenesis-promoting, embryogenesis-promoting or re generation-promoting genes tend to be lost. The transient expression of these genes, prior to their loss, can give the cells containing MC DNA a sufficient growth advantage, or sufficient tendency to develop into plant organs, embryos or a regenerable cell cluster, to outgrow the non-modified cells in their vicinity, or to form a readily identifiable structure that is not formed by non-modified cells. Loss of the DNA molecule encoding these genes prevents phenotypes from manifesting themselves that can be caused by these genes if present through the remainder of plant regeneration. In rare cases, the DNA molecules encoding plant growth regulator genes integrate into the host plant's genome or into the MC.

Alternatively, the genes promoting plant cell growth can be genes promoting shoot formation or embryogenesis, or giving rise to any identifiable organ, tissue or structure that can be regenerated into a plant. In this case, embryos or shoots harboring MCs directly after DNA delivery are obtained without the need to induce shoot formation with growth activators, or lowering the growth activator treatment necessary to regenerate plants. The advantages of this method are more rapid regeneration, higher transformation efficiency, lower background growth of non-modified tissue, and lower rates of morphologic abnormalities in the regenerated plants.

Determination of MC Structure and Autonomy in Adchromosomal Plants and Tissues

The structure and autonomy of the MC in adchromosomal plants and tissues can be determined by: conventional and pulsed-field Southern blot hybridization to genomic DNA from modified tissue subjected or not subjected to restriction endonuclease digestion, dot blot hybridization of genomic DNA from modified tissue hybridized with different MC specific sequences, MC rescue, exonuclease activity, PCR on DNA from modified tissues with probes specific to the MC, or FISH to nuclei of modified cells. Table 3 below summarizes these methods.

TABLE 3
Autonomous MC assays

| Assay | Details | Potential outcome | Interpretation |
|---|---|---|---|
| Southern blot | Restriction digest of | 1. Native sizes and pattern of | 1. Autonomous or integrated |
| | genomic DNA | bands | via CEN fragment |
| | compared to | 2. Altered sizes or pattern of | 2. Integrated or rearranged |
| | purified MC | bands | |
| CHEF gel | Restriction digest of | 1. Native sizes and pattern of | 1. Autonomous or integrated |
| Southern blot | genomic DNA | bands | via CEN fragment |
| | | 2. Altered sizes or pattern of | 2. Integrated or rearranged |
| | | bands | |
| | Native genomic DNA | 1. MC band migrating ahead | 1. Autonomous circles or |
| | (no digest) | of genomic DNA | linears present |
| | | 2. MC band co-migrating with | 2. Integrated |
| | | genomic DNA | |
| | | 3. >1 MC bands observed | 3. Various possibilities |
| Exonuclease | Exonuclease | 1. Signal strength close to | 1. Autonomous circles present |
| | digestion of | that w/o exonuclease | |
| | genomic DNA with | 2. No sgnal or signal strength | 2. Integrated |
| | detection of circular | lower than w/o exonucldease | |
| | MC by PCR, dot blot, | | |
| | or restriction digest | | |
| | (optional), | | |
| | electrophoresis and | | |
| | southern blot | | |
| | (useful for circular | | |
| | MCs) | | |
| MC rescue | Transformation of | 1. Colonies isolated only from | 1. Autonomous circles |
| | plant genomic DNA | MC plants wit MC, not from | present, native MC structure |
| | into E. coli followed | controls; MC structure | |
| | by selection for | matches that of the paretal MC | |
| | antibiotic resistance | 2. Colonies isolated only fo | 2. Atuonomouse circles |
| | genes on MC | MC plants with MCs, not | present, rearranged MC |
| | | from controls; MC | structure OR MCs integrated |
| | | strctureerent from parental MC | via centromere fragment. |
| | | 3. Colonies in MC modified | 3. Various possibilities |
| | | plants and and in controls | |
| PCR | PCR amplification of | 1. All MC parts detected | 1. Complete MC sequences |
| | various parts of MC | | present |
| | | 2. Subset of MC parts | 2. Partial MC sequences |
| | | detected | present |
| FISH | Detection of MC | 1. MC seqeuences detected, | 1. Autonomous |
| | sequences in mitotic | free of genome | |
| | or meiotic nuclei by | 2. MC sequences detected, | 2. Integrated |
| | fluorescence in situ | associated with genome | |
| | hybridization | 3. MC sequences detected, | 3. Both autonomous and |
| | | free and associated with | integrated MC sequences |
| | | genome | present |
| | | 4. No MC sequences | 4. MC DNA not visible by |
| | | detected | FISH |

Furthermore, MC structure can be examined by characterizing MCs rescued from adchromosomal cells. Circular MCs that contain bacterial sequences for their selection and propagation in bacteria can be rescued from an adchromosomal plant or plant cell and re-introduced into bacteria. If no loss of sequences has occurred during replication of the MC in plant cells, the MC is able to replicate in bacteria and confer antibiotic resistance. Total genomic DNA is isolated from the adchromosomal plant cells. The purified genomic DNA is introduced into bacteria (e.g., E. coli), and the transformed bacteria are plated on solid medium containing antibiotics to select bacterial clones modified with MC DNA. Modified bacterial clones are grown, the plasmid DNA purified (by alkaline lysis for example), and DNA analyzed, such as by restriction enzyme digestion and gel elcctrophoresis or by sequencing. Because plant-methylated DNA containing methylcytosine residues is degraded by wild-type strains of E. coli, bacterial strains (e.g., DH10B) deficient in the genes encoding methylation restriction nucleases (e.g., the mcr and mrr gene loci in E. coli) are best suited for this type of analysis. MC rescue can be performed on any plant tissue or clone of plant cells modified with a MC.

### I. Analyses of Transformed Plants

MC Autonomy Demonstration by In Situ Hybridization

To assess whether the MC is autonomous from the native plant chromosomes, or has integrated into the plant genome, in situ hybridizations can be used, such as FISH. In this assay, mitotic or meiotic tissue, such as root tips or meiocytes from the anther, possibly treated with metaphase arrest agents such as colchicines is obtained, and standard FISH methods are used to label both the centromere and sequences specific to the MC. For example, a Gossypium centromere is labeled using a probe from a sequence that labels all Gossypium centromeres, attached to one fluorescent tag, such as one that emits the red visible spectrum (ALEXA FLUOR® 568, for example (Invitrogen; Carlsbad, Calif.)), and sequences specific to the MC are labeled with another fluorescent tag, such as one emitting in the green visible spectrum (ALEXA FLUOR® 488, for example). All centromere sequences are detected with the first tag; only MCs are detected with both the first and second tag. Chromosomes are stained with a DNA-specific dye including but not limited to DAP1, Hocchst 33258, OliGreen, Giemsa YOYO, and TOTO. An autonomous MC is visualized as a body that shows hybridization signal with both centromere probes and MC specific probes and is separate from the native chromosomes.

Determination of Gene Expression Levels

The expression level of any gene present on the MC can be determined by several methods, such as for RNA, Northern Blot hybridization, Reverse Transcriptase-PCR, binding levels of a specific RNA-binding protein, in situ hybridization, or dot blot hybridization; or for proteins, Western blot hybridization, Enzyme-Linked Immunosorbant Assay (ELISA), fluorescent quantitation of a fluorescent gene product, enzymatic quantitation of an enzymatic gene product, immunohistochemical quantitation, or spectroscopic quantitation of a gene product that absorbs a specific wavelength of light.

Use of Exonuclease to Isolate Circular MC DNA from Genomic DNA

Exonucleases can be used to obtain pure MC DNA, suitable for isolation of MCs from E. coli or from plant cells. The method assumes a circular structure of the MC. A DNA preparation containing MC DNA and genomic DNA from the source organism is treated with exonuclease, for example lambda exonuclease combined with E. coli exonuclease I, or the ATP-dependent exonuclease (Qiagen, Inc.; Germantown, Md.). Because the exonuclease is only active on DNA ends, it specifically degrades the linear genomic DNA fragments, but does not degrade circular MC DNA. The result is MC DNA in pure form. The resultant MC DNA can be detected by a number of methods for DNA detection, such as PCR, dot blot, and Southern blot. Exonuclease treatment followed by detection of resultant circular MC can be used to determine MC autonomy.

Structural Analysis of MCs by BAC-End Sequencing

BAC-end sequencing procedures can be used to characterize MC clones for a variety of purposes, such as structural characterization, determination of sequence content, and determination of the precise sequence at a unique site on the chromosome (for example the specific sequence signature found at the junction between a centromere fragment and the vector sequences). In particular, this method is useful to prove the relationship between a parental MC and the MCs descended from it and isolated from plant cells by MC rescue, described above.

Methods for Scoring Meiotic MC Inheritance

A variety of methods can be used to assess the efficiency of meiotic MC transmission. In one embodiment of the method, gene expression of genes on the MC (marker genes or non-marker genes) can be scored by any method for detection of gene expression known to those skilled in the art, including visible scoring methods (e.g., fluorescence of fluorescent protein markers, scoring of visible phenotypes of the plant), scoring resistance of the plant or plant tissues to antibiotics, herbicides or other selective agents, measuring enzyme activity of proteins encoded by genes on the MC, measuring non-visible plant phenotypes, or directly measuring the RNA and protein products of gene expression using, for example, microarrays, northern blots, in situ hybridizations, dot blots, RT-PCR, western blots, immunoprecipitations, ELISAs, immunofluorescence and radio-immunoassays (RIAs). Gene expression can be scored in the post-meiotic stages of microspore, pollen, pollen tube or female gametophyte, or the post-zygotic stages such as embryo, seed, or progeny seedlings and plants. In another embodiment, the MC can de directly detected or visualized in post-meiotic, zygotic, embryonal or other cells in by detecting DNA (e.g., by FISH) or by MC rescue described above.

FISH Analysis of MC Copy Number in Meiocytes, Roots or Other Tissues of Adchromosomal Plants

The copy number of the MC can be assessed in any cell or plant tissue by in situ hybridization, such as FISH. For example, FISH methods are used to label the centromere, using a probe that labels all chromosomes with one fluorescent tag, and to label sequences specific to the MC with another fluorescent tag. All centromere sequences are detected with the first tag; only MCs are detected with both the first and second tag. Nuclei are counter-stained with a DNA-specific dye, such as DAPI, Hoechst 33258, OliGreen, Giemsa YOYO, and TOTO. MC copy number is determined by counting the number of fluorescent foci that label with both tags.

Induction of Callus and Roots from Ad Chromosomal Plants Tissues for Inheritance Assays

MC inheritance is assessed using callus and roots induced from transformed plants. To induce roots and callus, tissues such as leaf pieces are prepared from adchromosomal plants and cultured on a MS medium containing a cytokinin, e.g., 6-benzylaminopurinc (BA), and an auxin, e.g., α-naphthaleneacctic acid (NAA). Any tissue of an adchromosomal plant can be used for callus and root induction, and the medium recipe for tissue culture can be optimized using procedures known in the art.

Clonal Propagation of Adchromosomal Plants

To produce multiple clones of plants from a MC-transformed plant, any tissue of the plant can be tissue-cultured for shoot organogenesis using regeneration procedures already described. Alternatively, multiple auxiliary buds can be induced from a MC-modified plant by excising the shoot tip, rooting the tip, and subsequently growing the tip into plant; each auxiliary bud can be rooted and produce a whole plant.

Scoring of Antibiotic- or Herbicide-Resistance in Seedlings and Plants (Progeny of Self- and Out-Crossed Transformants

Progeny seeds harvested from MC-modified plants can be scored for antibiotic- or herbicide resistance by seed germination under sterile conditions on a growth media (for example, MS medium) containing an appropriate selective agent for a particular selectable marker gene. Only seeds containing the MC can germinate on the medium and further grow and develop into whole plants. Alternatively, seeds can be germinated in soil, and the germinating seedlings can then be sprayed with a selective agent appropriate for a selectable marker gene. Seedlings that do not contain MC do not survive; only seedlings containing MC can survive and develop into mature plants.

Genetic Methods for Analyzing MC Performance

In addition to direct transformation of a plant with a MC, plants containing a MC can be prepared by crossing a first plant containing the functional, stable, autonomous MC with a second plant lacking the MC.

For example, pollen from an adchromosomal plant can be used to fertilize the stigma of a non-adchromosomal plant. MC presence is scored in the progeny of this cross using the methods outlined above. In the second embodiment, the reciprocal cross is performed by using pollen from a non-adchromosomal plant to fertilize the flowers of an adchromosomal plant. The rate of MC inheritance in both crosses can be used to establish the frequencies of meiotic inheritance in male and female meiosis. In the third embodiment, the progeny of one of the crosses just described are back-crossed to the non-adchromosomal parental line, and the progeny of this second cross are scored for the presence of genetic markers in the plant's natural chromosomes as well as the MC. Scoring of a sufficient marker set against a sufficiently large set of progeny allows the determination oflinkage or co-segregation of the MC (or lack thereof) to specific chromosomes or chromosomal loci in the plant's genome. Genetic crosses performed for testing genetic linkage can be done with a variety of combinations of parental lines as are known to those skilled in the art.

### IV. EXAMPLES

The following examples are for illustrative purposes only and should not be interpreted as limitations of the claimed invention. There are a variety of alternative techniques and procedures available to those of skill in the art which would similarly permit one to successfully perform the intended invention.

In summary, Examples 1-7 illustrate the identification of cotton centromere-localized sequences. In a first approach (illustrated in Example 1) cotton repetitive sequences were identified using methods that enrich for the highly repetitive and extensively methylated centromere DNA. First, the investigators digested G. hirsutum ™-1 genomic DNA with a methylation-sensitive restriction enzyme, then isolated fragments larger than 8 kb, to enrich for sequences that were extensively methylated and thus would not be cut by the methylation-sensitive endonucleases (Luo, Hall et al. 2004). The investigators then used these large fragments as probes to isolate clones from a G. hirsutum Bacterial Artificial Chromosome (BAC) library. To determine whether any of these clones derived from the centromere, the investigators used Fluorescence In Situ Hybridization (FISH) on mitotic spreads from cotton roots to look for localization to the cytological centromere, as defined by the primary constriction on each chromosome. The investigators tested 16 BACs representing highly methylated genomic DNA and found none that hybridized to the region of the cytological centromere.

In a second approach (Example 2), the investigators used tandem repeats as FISH probes to query the G. hirsutum genome because tandem repeats have been associated with the centromere in many plants. The tandem repeats were selected from those previously described in the literature, or by bioinformatic analysis of cotton genomic sequences to find simple tandem repeats. None of the repeats examined displayed obvious localization to the cytological centromere. These results compelled the investigators to use a different strategy to find centromere-associated sequences in the highly repetitive and methylated cotton genome.

Thus, as a third and different approach, the investigators reasoned that if, like other plants, the cotton centromere comprises mainly repetitive DNA, then it should be over-represented in randomly generated cotton genomic survey sequence. To test this theory (Example 3), the investigators downloaded G. hirsutum genomic sequences from the public databases and assembled them into contigs using low stringency parameters. The contigs containing the most overlapping sequences (the “deepest reads”) were selected for further analysis. The deep read sequences were grouped into classes by homology and a representative sample from each class was used as a FISH probe to determine possible localization at the cytological centromere. This approach identified one contig containing an LTR retrotransposon that specifically hybridizes to the primary constriction of all 52 chromosomes in G. hirsutum ™-1. Following the nomenclature of other centromere-associated elements, the investigators have designated this element Centromere Retroelement Gossypium, (CRG).

### Example 1

### Bioinformatics Identification of Tandem Repeats in G. hirsutum

In a first approach, the investigators compiled Gossypium repetitive genomic DNA as candidate probes for hybridization with the BAC libraries (Table 4). Gossypium sequence was extracted from GenBank and tandem repeats identified using publicly available repetitive DNA sequence finder software. The identified Gossypium repeat elements were PCR-amplified and used as fluorescence in situ hybridization (FISH) probes to identify repeat elements that localized to Gossypium chromosomes.

The investigators initially identified the sequence elements listed in Table 4 (SEQ ID NOs:21-27). Each sequence represents a different repetitive DNA sequence from G. hirsutum.

TABLE 4
Cotton repetitive genomic sequence and BAC library probes

| | | Repeat | |
|---|---|---|---|
| Probe | GenBank | length (bp) | PCR Primer Sequences (5′ to 3′) |
| ChrGh-1 | BH023123, | 195 | CRJM105: agggtcataa gacaagcttc |
| (SEQ ID NO: 21) | BH023124, | | agtcttggg (SEQ ID NO: 28) |
| | BH023255, | | CRJM106: caataataag ttggctaaag |
| | BH023337, | | attttcatag gc (SEQ ID NO: 29) |
| | BH023338, | | |
| | BH021849 | | |
| ChrGh-2 | BH022338 | 96 | CRJM107: atatataca ctttcacat tcatcacat |
| (SEQID NO: 22) | | | cgg (SEQ ID NO: 30) |
| | | | CRJM108: gtgataggg ccgaatgg ccgatgtga |
| | | | tg (SEQ ID NO: 31) |
| ChrGh-3 | BH022651 | 133 | CRJM109: attttggtat ttagatgtt cttttggt |
| (SEQ ID NO: 23) | | | c (SEQ ID NO: 32) |
| | | | CRJM110: ataacaatt ttattatat aaaaccgtg |
| | | | agc (SEQ ID NO: 33) |
| ChrGh-4 | BH022299 | 148 | CRJM111: tggaagtttaaagtagcttatgccaaac |
| (SEQ ID NO: 24) | | | (SEQ ID NO: 24) |
| | | | CRJM112: caagctttat gcaataaatt |
| | | | gtaaaaagg ag (SEQIDNO: 25) |
| ChrGh-5 | CL863894 | 225 | CRJM113: tcttatctcc ctgaagttg cagtggag |
| (SEQ ID NO: 25) | | | (SEQ ID NO: 36) |
| | | | CRJM114: ctttaacttc aacctgctc cactgctac |
| | | | (SEQ ID NO: 37) |
| ChrGh-6 | CLS640S9 | 108 | CRJM115: catgtaagac catgccaagg catggc |
| (SEQ ID NO: 26) | | | (SEQ ID NO: 28) |
| | | | CRJM116: gaaccttttc cttatagtaa |
| | | | ctcatcaat gcc (SEQIDNO: 29) |
| ChrGh-7 | BH0216-8 | 97 | CRJM117: cttacatct cttaagcta aatagtatg |
| (SEQ ID NO: 27) | | | tgatgaa (SEQ ID NO: 40) |
| | | | CRJM118: cagaacata ccaagcaac ccataaaac |
| | | | (SEQ ID NO: 41) |

In a variation of the tandem repeat approach that allowed the investigators to more quickly identify candidate centromeric tandem repeats, the rationale was used that tandem repeats that are specific to the centromere will be over-represented in sequenced genomes. G. hirsutum, an AD tetraploid, is the result of interspecific hybridization of “A”—African/Asian and “D”—American genomes. The genome comprises 52 chromosomes, and the haploid genome size is about 2.5 Gb. The investigators estimated that centromeric tandem repeats would constitute about 5% of the genome.

All cotton sequences available in the public databases were downloaded and assembled into contiguous arrays (“contigs”) using low stringency assembly algorithms. Those contigs having the most alignments (“deepest reads”), and presumably harboring centromeric repeats, are then selected for further analysis.

The investigators downloaded all nuclear genome cotton sequence data available from the public dataset “NCBI dbGSS” as of March 2009 (National Center for Biotechnology Information; National Library of Medicine; Bethesda, Md.) and used Tandem Repeat Finder software (Benson 1999), PHRAP (version 0.990329) (Bonfield and Staden 1995) and the NCBI Align Tool (Altschul, Gish et al. 1990).

The parameters for running PHRAP were:

Custom:
a. Maxgap 30
b. Revise_greedy
c. Shatter_greedy
d. Penalty −8

Manual ((−) more stringent)
e. Penalty −9 or −6
f. Penalty −2
g. Maxgap 30
h. Bypasslevel 1
i. Node_seg 8->optionals
j. Node_space 4->optionals
   i. Repeat_stringency 0.95->stringency
k. Forcelevel 0->non-stringency
I. Minmatch 14->stringency
m. Minscore 30 or 40->stringency
n. Bypasslevel 0

The contigs were visually inspected for any collapsed contigs. For all collapsed contigs, the PHRAP parameters were adjusted, and the assembly rerun. Non-collapsed contigs were then aligned manually or by using Align. The Tandem Repeat Finder was run on the deepest-read contigs using default parameters. After identifying those contigs with tandem repeats, the contigs were inspected to identify consensus sequences that were repeated more than two times and their coverage exceeded 100 bp. Those contigs that had deep reads and low complexity were analyzed for repetitive structure.

The identified contigs were then analyzed for alignment using BLASTN algorithms in GenBank to identify homology with other plants, discarding repeats that were identified in mRNAs.

Using this procedure, one previously identified (194 bp) and two novel tandem repeats (210 bp and 100 bp) were found; their consensus sequences are presented in Table 5.

TABLE 5
Tandem repeats identified in G. hirsutum

SEQ ID NO: 90 (CrGh1)
tttacacccc tagaagactg cctaagaaaa ttattagcaa cttattattg aaggtcataa 60
gacaagtttg agtcttgggg cattgaaacc atgttatatg cacatgtatc atcaccatca 120
aagaagctta attcatttta caaacagtga ctccactata gcgagcccaa ctagctcaca 180
tgtaacttga cttg 194
SEQ ID NO: 91
ccttccatca aagtggaaat gccagacttc gatttgttct ctgacaatac agagacgaca 60
aatcatctta gatttgcttc atcgtaaaca catgaaagcc aaatcagcta tatcttcgat 120
ctgctcccta ccgaatacag agagataaga tctgtcatct tggatctgct tcagcataaa 180
catctgaagg taagatctgc tatgtgtctg 210
SEQ ID NO: 92
ggcgatgcag tggaacagat taaagccaca tcggtgaatc ttgcttcccc gacattgcag 60
ttaaaaagat taaagctaca acagcgaatc ttactcccca 100

SEQ ID NOs:90-92 were then subjected to further analysis to determine if these sequences represented centromeric sequences.

Library Interrogation and Data Analysis

The BAC clones from the libraries (see Example 3 for library details) were spotted onto filters, and the filters were hybridized with a subset of the probes in Tables 4 and 5 to identify specific BAC clones that contained DNA from the group of sequences represented by the probes. The specific probes used were: CrGh1 (SEQ ID NO:90), and pXP1-80 (a Gossypium barbadense repeat sequence, Genbank accession number AF060649.1,

(SEQ ID NO: 99)
cccttggccg tgcgggagcc tctagcacca tcggcacctt ggtgcttgga tgtccgagaa 60
cctcgcgcac catcgacaac ctagg, 85

which had been purported to be a centromeric sequence (Zhao, Wing et al. 1995; Zhao, Si et al. 1998)). The probes were hybridized to the BAC filters using standard molecular biology techniques to identify BACs that containing these tandem repeats.

### Example 2

### Analysis of Tandem Repeats Identified Using Bioinformatics

The investigators designed primers to amplify the tandem repeats represented by the consensus sequences of SEQ ID NOs:90-92, shown in Table 6.

TABLE 6
Primers for amplifying tandem repeats of
SEQ ID NOS: 90-92

| SEQ | | |
|---|---|---|
| ID NO | Sequence | Target |
| 100 | gagtcttggg gcattgaaac | SEQ ID NO: 90 Forward |
| 101 | gagctagttg ggctcgctat | SEQ ID NO: 90 Reverse |
| 102 | agtggaaatg ccagacttcg | SEQ ID NO: 91 Forward |
| 103 | gctgatttgg ctttcatgtg | SEQ ID NO: 91 Reverse |
| 104 | ccccgacatt gcagttaaaa | SEQ ID NO: 92 Forward |
| 105 | tggggagtaa gattcgctgt | SEQ ID NO: 92 Reverse |

Amplified fragments were then prepared as probes for FISH analysis of G. hirsutum chromosomes. If the sequences represented centromeric sequences, then the probes would localize to cytological constrictions that correlate to centromere structures in condensed chromosomes at metaphase on every chromosome (or at least to the A or D sub-genomes).

FISH was performed essentially as described (Kato, Lamb et al. 2004) using probes labeled with ALEXA FLUOR® 488 (green) or ALEXA FLUOR® 568 (red) dyes (Molecular Probes). Probes were hybridized to chromosome spreads prepared from fixed cotton root tips. To prepare the spreads, cotton root tips were fixed in Carnoy's fixative (3:1 ethanol:glacial acetic acid) and stored at −20° C. Stored root tips were washed in distilled water, then the root caps were rinsed in distilled water, and the meristematic regions dissected out. Protoplasts were prepared enzymatically, and protoplasts spread by dropping onto slides. Following hybridization, slides were counterstained with DAPI (0.04 mg/ml) and the slides examined under a microscope using a Zeiss Axio-Imager equipped with rhodamine, FITC, and DAPI filter sets (excitation BP 550/24, emission BP 605/70; excitation BP 470/40, emission: BP525/50; and excitation G 365, emission BP 445/50, respectively). Grayscale images were captured in each panel, merged, and adjusted with pseudo-color using Zeiss AxioVision (Version 4.5) software.

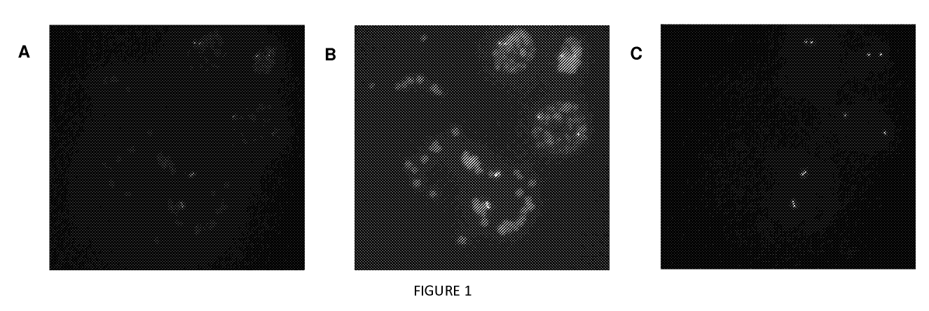

Surprisingly, the investigators did not observe localization at centromeric constrictions at every centromere. FIG. 1 shows the results of one of these experiments and shows a co-localization of the tandem repeat of SEQ ID NO:90 with a probe prepared to identify CrGh1. FIG. 1A shows the signal from the probe used to identify the localization of SEQ ID NO:90. FIG. 1C shows the staining that is observed when a probe to CrGh1 (also SEQ ID NO:90). When the images are overlaid, the signals perfectly merge (FIG. 1B), and only few chromosomes are stained at the centromeric constriction.

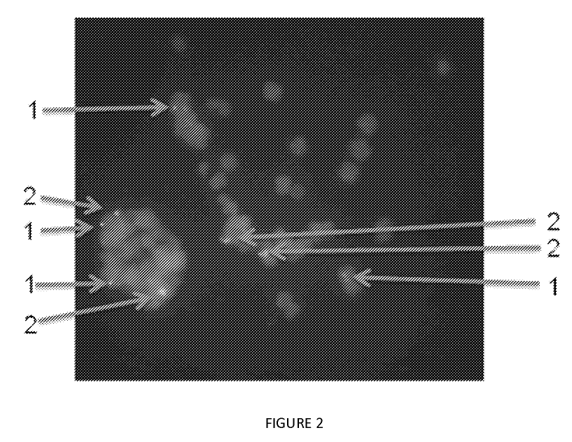

While the results for the 210 bp repeat (SEQ ID NO:91) were not the same as those for SEQ ID NO:90, the result was equally surprising. FIG. 2 shows the results of a co-localization experiment. The probe for SEQ ID NO:91 1 only localized to few centromeric constrictions, although closer observation showed that the probe stained throughout the genome. As expected in view of the results shown in FIG. 1, the probe for the 194 bp repeat (SEQ ID NO:90) 2 also localized to one centromeric constriction. However, there was no overlap between the two probes in their staining pattern as shown in FIG. 2. Only diffuse, non-centromeric staining was observed when sample were probed with the 100 bp repeat (SEQ ID NO: 92) (data not shown).

While it would be expected that tandem repeat sequences commonly present in higher plant functional centromeres, it is possible that the repeats of SEQ ID NOs:90, 91 and 92 can function as centromeres, conferring autonomous segregation in mitosis and meiosis.

### Example 3

### G. hirsutum Centromere Identification Using a Methylation Approach

In another approach, methylation sensitive technique (e.g., as described in U.S. Pat. No. 7,132,240 and by (Luo, Hall et al. 2004)), was tried to identify cotton centromeres. G. hirsutum L. TM-1 genomic DNA was prepared and digested with the methylation-sensitive endonuclease, BfuCl (New England Biolabs; Ipswich, Mass.). Fragments greater than 10 kb (representing methylated fragments) were purified from agarose gels after electrophoresis, as were unmethylated, smaller (less than 3 kb) fragments. These fragments were then used to probe the BAC library that had been constructed from G. hirsutum L. TM-1 genomic DNA to identify BACs containing methylated, methylated and unmethylated, and unmethylated repetitive DNA. Positive clones included those BACs that hybridized with methylated, but not unmethylated probes. The BACs identified by this method were then used as probes in FISH analysis to determine chromosomal localization.

The BAC library used in these experiments was constructed from G. hirsutum L. TM-1 genomic DNA was isolated and was digested with the methylation-insensitive Mbol restriction enzyme. The resulting BAC library had 43,008 BACs, representing about 1.8-fold haploid genome coverage.

To interrogate the libraries, two probes were prepared by digesting cotton genomic DNA. Probes were prepared by digesting cotton genomic DNA with the methylation-sensitive restriction endonuclease BfuC1 and the resulting fragments separated by agarose gel electrophoresis. Fragments were purified from two size ranges: (1) high (>10 kb) and (2) low (<3 kb). The high fragment pool contained sequences that were cut less frequently by the methylation-sensitive restriction endonuclease; therefore, this pool was enriched for highly methylated regions of the genome. The low fragment pool contained sequences that were cut by the methylation-sensitive restriction endonuclease; therefore, this pool was enriched for less-methylated regions of the genome. Two duplicate copies of the cotton genomic BAC library were probed in parallel with the high- and low-methylation fragment pools. Probes were labeled to incorporate ³²P dCTP and were hybridized to filters at 65° C. in 5×SSC, 0.5% SDS, 25 mM sodium phosphate, 5×Denhardt's, overnight, then washed in 0.5×SSC, 0.5% SDS. The filters were washed at 65° C., first for 15 minutes, and then two washes of 1-2 hours each. Signal was detected by exposing the filters to a phosphoimager screen overnight. BAC clones were visually scored for hybridization to each probe, and clones were selected based on strong hybridization signals to high methylation probes and little or no hybridization to the low methalyzlation probes.

When the investigators observed samples prepared using FISH and probed with BACs 7908, 68C15, 20L23 and 61P1, which had been identified as candidates containing centromeric sequences, they were surprised to see that all probes failed to give characteristic centromeric staining. Instead of strong, centromeric constriction staining, signal was observed to be diffuse over the entire chromosomes. One BAC, BAC_{—}9E14, harboring pXP1-80 (SEQ ID NO:99) (Zhao, Wing et al. 1995; Zhao, Si et al. 1998), did show a more localized staining pattern, but further FISH analysis indicated that the sequence co-localized with NOR 185 rDNA and was therefore determined not to be centromere DNA (data not shown). This result was also surprising: the pXP-180 repetitive element (SEQ ID NO:99) shares many of the characteristics of a centromere repeat in that it is a tandem repeat observed to be present in all tested cotton species and is similar in size to other known centromere repeats (Hawkins, Kim et al. 2006).

### Example 4

### Identification of Cotton Centromeric Sequences Using an Alternative Approach

While the bioinformatics tandem repeat approach and the methylation approach had both surprisingly failed to identify centromeric sequences, the investigators reasoned that centromeric sequences would be highly represented in the genome, as they had reasoned in the bioinformatics tandem repeat approach (Example 1). However, in this case, the investigators looked for sequence contigs that did not have tandem repeats as previously determined in Example 1, but did have deep reads. Such contigs should have high-copy sequences that are not tandemly arrayed. Using the same conditions to assemble contigs as outlined in Example 1, the investigators examined contigs having deep reads lacking tandem repeats, designed PCR primers to amplify fragments within the contigs, and used these fragments as probes in FISH analysis of G. hirsutum mitotic metaphase chromosomes.

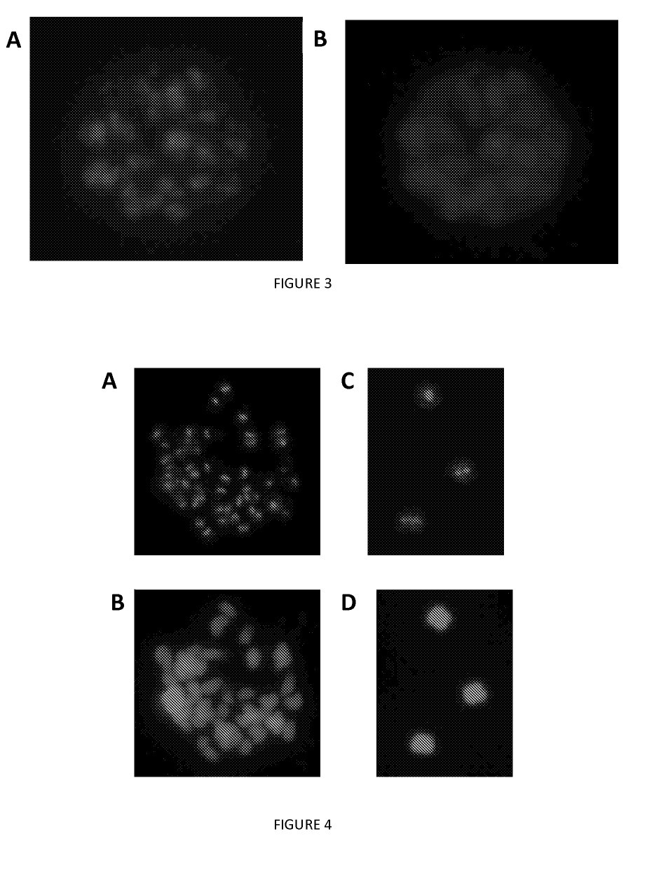

Two contigs were initially analyzed, 4198 (consensus sequence: SEQ ID NO: 93) and 4199 (consensus sequence: SEQ ID NO:94). SEQ ID NOs:93 and 94 are shown in Table 7, as well as the probes used in FISH analyses. When analyzed by FISH (as described in Example 2) a probe (SEQ ID NO:106) specific for contig 4198 (SEQ ID NO:93) gave only diffuse staining (FIG. 3A). FIG. 3B shows the same chromosome preparation counterstained with DAPI, which stains whole chromosomes.

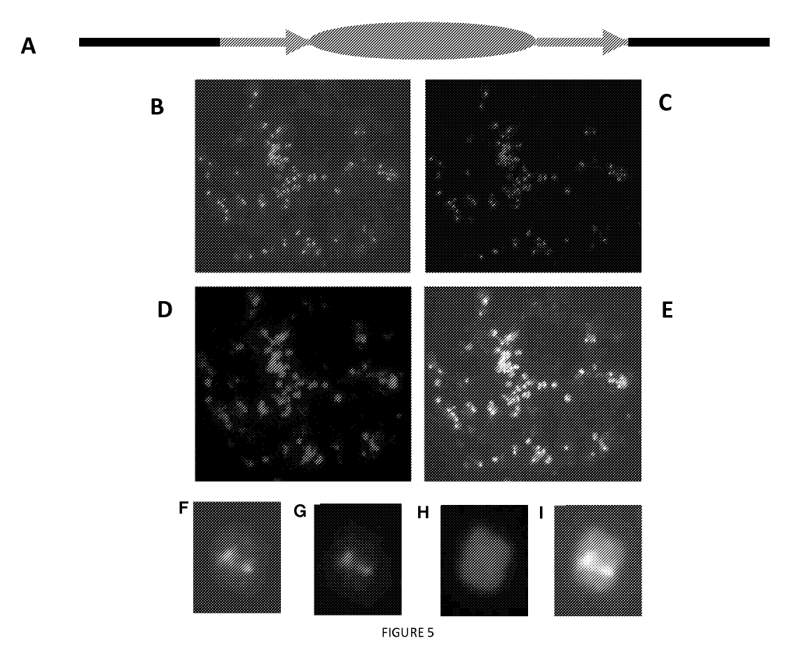

However, as shown in FIG. 4A, when a probe (SEQ ID NO:107) specific to contig 4199 (SEQ ID NO:94) was used for FISH analysis, every chromosome was stained in the centromeric region as demonstrated by punctuate staining at the centromeric constriction; FIG. 4B shows the same preparation as FIG. 4A counterstained with DAPI to show the entire chromosomes. FIGS. 4C and 4D show magnified images of fields of FIGS. 4A and 4B, respectively.

Because every centromeric constriction is labeled by the contig 4199 probe, this observation suggests that contig 4199 comprises centromere DNA.

To understand the variability within the contig, the investigators determined that the sequences in the contig 4199 were 79% to 99% identical to the consensus sequence of SEQ ID NO:94.

TABLE 7
Consensus sequences for contigs 4198 (SEQ ID NO: 93) and 4199
(SEQ ID NO: 94) of G. hirsutum and FISH probe nucleic acid sequences

SEQ ID NO: 93 (contig 4198)
tttttttttt ttatgtgagt atttgtgaga aaaaagaatt ttccacaatc aaaaaaaaaa 60
aatttacccc ttttttgtgg agaaagggta ttttttttaa aagagacatt ttcggatttt 120
aaatcttttt ttcaaacact ttacaccccc cttgagatta accaaaaaaa ttaattgggc 180
caaggcgcgt tggttttttt tccaatgggg ggaaactttt tttggaaggg gttgattaaa 240
tttaaacaca taggaaacaa ttccccattt ggggattttt tgagtatttg ctggatttta 300
cagcaagctc attcattacc aaatcagtac cctcgggatt taaccgaata tagctctcgt 360
tcaaatgctt cgggacaata gcccggtttt atnaactcac acaatgcctt tcggacttaa 420
cccggattta acaatcgcac gaatgccttc gggacttaac ccggatttaa cactcgcacg 480
aatgccttcg ggacttaacc cggatttaac aactcgcacg aatgcttcgg ggacttaacc 540
cggatttagt atctagcaca aaggccttcg gatcttaatc cggatatatt cacttagcac 600
aaagccttcg ggacttagcc cggacagcat tcaattaatc atgcacatct aacaataatt 660
catagcacat tcatatttca ttttcgttta cgaaactcaa acacaaggca catattgtcc 720
ttgcacattc ggctcaatag ccacacatag agcatgattt aatcacatcg aaatttaaga 780
tctcttactc aagaacttac ctcgggtgtt gtcgaacgat tccgctagct attcaaccac 840
tttttccttc cctttatcgg ttttatttcc cctttgctct tgagcttaat caaacaaata 900
aattgatttc atcattttag gcatcaaaag atgaacacaa ggcacttagc ccatatttat 960
acattagaca ttaaagtctc atacatgcaa aaatcatgca tcaacacaac atattagcta 1020
atttctttcc ccttggccga atatgcatgt ccatttttgg ggtcgatttc aacacttaat 1080
acacacatat acacactagt aaagcatcct cccccttttc atcgatttaa cacatgcatt 1140
gctcatcaac atgcaaagtt acattcggcc ttagcacaca tcttgctagc cgattcttct 1200
ccctttagca accaatgcac atatgtgctc acacaaaaat gctaaaaagg aggttcaaga 1260
atcatcaagc catcatcaca tgcatcatta acaagcttca tattttgcat gcaatggcat 1320
taacacaacc tccacctagg ccgaatctta actcatcctc atgcctcatc accacaacat 1380
caaacatcaa ccaagaatga ttcatccatg gtcaagtgcc atttccatca catagcaaga 1440
tttagaccat gggttaggta gaactcaagc taacaactaa aacatgcatg cctctcatgg 1500
aacatcatca aacatacctt agcagcctag ctacatgcat ggccgaatct cttcaccttt 1560
cttcttcttt cctccttaaa atttttggcc aaggatgaac caaaggatga gaaatttttt 1620
tctttgtttt tctttctagt ttttggcaag catgaagatg agaaaaggat gaacacaaat 1680
ttttctcctt tcttctcttt agctcacggc aatgggggga caaacaccac acacacattt 1740
ttttttcttt tgttttccat ttctttatta cccatactcc ttattttatt cttccactaa 1800
caaaacatgt ttcatgacat gttttgccca tccttccttg tcatggccgg ccactactca 1860
ttaggggggg gaatttgaca tgcaagtccc ccctttgtcc acatgcacta gtaggtcctc 1920
acacattgac ctatcacatt ttagaatttt ctcacataag tcctattgac taaattcaca 1980
tgaaatcaac caaattgaag cttgaaattt tcacacattc ataattacat attctacaca 2040
ataagtatca cattcaaaca tttcggtgac tcggtttagc ggtcccgaaa ccacttcccg 2100
actagggtca actttgggct gtcacaactc tcccccactt aagaaaattt tcgtccccga 2160
aaatcttacc ggtaaatagg tttgggtatc gttctttcat cgagctctcg gtctcccaag 2220
tagcttcctc gatcccgtgt ttgagccata acacctttac tagcggaact cttttgtttc 2280
gcaactcctt cacttcacga gctaggatac gcatcggttc ttcttcatag ctcatgtcgt 2340
cttgaatttc aacctctgat gggctaatta tatgcgatgg atcagatcga tagcgtcgaa 2400
gcatcgagac atgaaagacg tcatgaatct tttcaagctc cgggggcaaa atcaatctat 2460
acgcaaccga gccaactcgc tcggagattt cgtacggccc aatgaatctt gggctcaact 2520
tgcccttacg gccgaacctg agtatctttt tccaaggtga aaccttaagg aacactttgt 2580
ctcccacatg atactcgatg tcttttcgtt tcaaatctgc gtacgatttc tgacgatccg 2640
tggctacctt cagactttca cggattacct ttaccttctg ttcagcatct ctaatcaaat 2700
caactccgaa aattttactt tcaccgagct cggtccaaaa caatggtgta cggcatttac 2760
gaccgtacaa agcctcgtaa ggtgccatct taatacttga ttgaaaacta ttgttgtaag 2820
cgaattcaat caaaggtaaa taccgttccc atgaactact gaactcgagg atgcaacatc 2880
tcaacatatc ctcaagtatc tgaattatcc gctcggattg accatcggtt tgggggtgaa 2940
aagcagtgct aaaatgcaac ttggtaccca gagcttcttg caatttcttc caaaatcgtg 3000
aggtgaatct cggatctcta tccgacacga tagaaacagg taccccgtgt aatctcacaa 3060
tttgataaac gtacaattca gctagtttat ccaatgaaaa atccgtacgt acggggatga 3120
aatgagccga cttagtcagt ctatcaacaa taacccatat cgcatccttc ttacttgctg 3180
acaaaggcag tccggacaca aagtccattg tgactcgatc ccatttccac tcgggtatca 3240
tgatcggctg gagtaatcct gaagacactt gatgttccgc gtttcacttg ttgacatatt 3300
aaacatctcg aaacaaagtc agagatgtcc cgtttcatac catgccacca aaattgacgt 3360
ttcaaatcgt tgtacatttt cgtacttccc cgggtgaatt gacattcggc tacaatgggc 3420
ttcgttcaga atcatcaaaa tgagttccga attccttgga acacacaaac gacttctgaa 3480
cctcaaacaa tcatcatcac caatttgaaa ctccgattcc ttattcggaa cacactcagc 3540
ccgttttgca accaattcat catcaacttt ctgagcttca cgaatttgat gaatcaataa 3600
tggtttggct tttaattcag ctactaacac attgtcaagt agaacagacc agtgcacatt 3660
catcgctcgt aaagcaaaca atgatttccg gcttaaggcg tccgcaacca cattagcctt 3720
tcccgggtgg taatcaatga caagctcgta atctttcaac aactcaagcc aacgtctttg 3780
tcgcagattt aagtctcttt gagtcatcaa atatttgaga cttttgtgat ccgaaaatac 3840
atggcacttt tcaccaaata agtaatgtcg ccatattttt aaagcaaata cgatggcagc 3900
tagttcaaga tcatgggtcg gataattttt ctcatgtggc tttaattgtc tcgacgcata 3960
ggccacaact cgaccttctt gcatcaatac gcaacccaac ccaagtaggg atgcgtcact 4020
ataaatgaca aactcttttc ctgattcggg ttgcactaaa aatggaagct ccatccaaaa 4080
aaatttttca gttgaatcta aaccttttct gacatttctc cgtccattcg aacttaacat 4140
ccttttgaag tagcttcgtc attggtgtgg ctatcatcga gaaacctttt acaaatcgtc 4200
ggtaataacc ggtgagcccc aggaagctcc gaacttcggt aatatttctt ggaggcttcc 4260
agttaagtat ggctgaaatt ttgctcgggt caactcgaat acccgatgcg gatcccacat 4320
gacccaaaga tgctaacctc ttttaaccag aactcacact tactgaactt agcatataac 4380
tgcttatccc gtaaaattta caacactaat ctcaggtgct cagcatgttc agtctcatct 4440
cttgaataga ccaagatgtc atcaatgaac acaactacga accgatccaa atatgggtct 4500
gaggatccga ttcatcaaat ccataaatac cgcaggggca ttagtgagcc aaaacggcat 4560
cactaagaac tcgtagtaac agtatctagt tctaaaagca gttttgggta tatccgaatc 4620
tcgaattcgc aattggtaat aactcgatct caaatctatc tttcaaaaca ctgaggctcc 4680
ctttagttga tcaaacaaat catcaatacg cggtaacgga tatttattct ttatcgtcac 4740
tttattcagc taacgatagt caatgcacaa cctcatggtt ccgtccttct ttttcacaaa 4800
caatactggt gcaccccaag gtgagaaact tggtcgagtg aaacctctat ccgtcaactc 4860
ttgcaactga gctttcaaat gttttaactc ggttggttcc atacgatacg gagctatcga 4920
aattggcata gttccaggta caagctcaat accaaactct atctcccgaa caggtggcaa 4980
acccggtaac tcttcaagaa acacatccgg gtattcacag atcactcggc accgattcgg 5040
gtttcttttc taactccttg tcatcaagta catacgcgag gtatgcttcg cacccttttc 5100
ttacatattt ctgggccaac attgccaata ttacagctgg caatcccctt aagtccgtag 5160
actcaattcg aattatctcg ttatttgcgc acctcaaatc aatagtcttt tcttttgcaa 5220
ttcacaactg catcatgaac ggtcaaccaa tctttttcga gaataacatc aaattcatca 5280
aacggaaaaa gcatcaagtc cgccggaaaa cgggaacctc gaattactag ggggcatttc 5340
ttacacactt tgtcgacaag cacgtaacga cccaagggat ttgacaccag aattacgaac 5400
tcagtagact caataggtaa agtcttactg gatgctaagg tttcgcatat ataagaatga 5460
gtagaaccag ggtcaatcaa agcaatcaca ttagtatcaa agagagtaaa agtaccggta 5520
ataacatctg gcgaggaagc atcctcgcgt gcgcgtatag cataagcctt agcaggagcg 5580
cgagcctcgg atctagtcgt agcatctata gatcctctct gaccacccct agcattgccc 5640
gtatttctag atggtctacc tcgaacagtg gtagcacccg gtttctcgct ttgatttata 5700
ttctgttcag acaacctcgg gcaatccttt attgaagtgg tcaactgatc cgcacttgta 5760
acagggagcg atcatggaat ctacaactcc ccgaatgcca tttaccacaa tactggcact 5820
ctgctctgtc tcgacgatca tttccaccac tggcgaccga agtgactcgt gcactcacag 5880
ggggtcgatc gcgatctcgt ctagaaaagc cagaaagtgt ttctagatcg gcctaaatca 5940
tctctaaaat ttcttcgatg actgtttgaa agggctttcc cgaagatctc ttacgaaact 6000
cttttgctcc tacatcagct ttccttttat ccttactgag ctcctcggct ttgcaagctc 6060
gctcgacaag taccacaaat tctcggattt ctaaaatgcc aacacacagc ttttatatca 6120
tcattcagtc catcctagaa ccatttacac atcacggcct ctgaagaaat gcattctcgc 6180
gcgtatcggc taagcctcac aaattttcgt tcaatagtcg gtaaccgaca tggaaccttg 6240
tttgagttca agaaattcct ttcgtttttg gtcgatgaat ctatgactga tatacttctt 6300
tcaaaactca aaaaaaagaa ctcccaagtt acttactctc tgggcacaac agaagtcaac 6360
gtattccacc aatagtaggc agaatcatgt agcaaggaga taatacactt taggcactca 6420
tcgggtgtgc aagatagctc atcgagtacc cgaatagtgt tgtccaacca aaattcagct 6480
tgctcggcat catcgctatc cgtagcctta aattcagtag ccccgtgttt tcgaattctg 6540
tcaattgggg gcttacttga ccttatttgg tcagttgccg gaggtattgt aggtgcgggg 6600
gtggcattag ttcggaatgg aggttgctgc gaacagccgt attagttcga atgtattggt 6660
tgaaacaatc attcatcatg ctataaaaag cttgtctagc ttcatcattt ggattactag 6720
caataggttg agagtccgcc ggagctgtcc cttgtgcggg agcaggcgct acactctcaa 6780
gatcatcagc taccgctcgg tcgggattgg gatccattac tataaataaa cacatttgca 6840
aatgtcagaa atcaccacac tatcaattaa tcacataaaa tggcatgtat agttagaccc 6900
caacacatta cggtagtcct agaaacgact accaccgtag ctctgatacc aatcaaatgt 6960
aacaccccga acccgagatc gtcaccggag tcggacacga gatgttaaca aactttgaaa 7020
aaaatttttt ccagacactg cccagtctga gtactagtcg cttcaaaaat catatcttga 7080
gtttcacaac tcaaaaatca gttttgtatt tttccctgaa acaagactca tgtccccacc 7140
tatggatttt tttctggaat ttttggttgg gccaactagt acagtttatt agtcaaagtc 7200
tcccatgtta caggggtcga ctacactgac cttttcccat tacgactggg atatctctct 7260
gcacagagct tcaatactga tgccgtttgt ttctatggaa actagactca gagaggaatc 7320
catacatata tggtatgacc cctaattatc tctggtcaat ttatagtgaa tttccaaagg 7380
cggaacagtg aatcccagaa actgttctgg ccctgttcca caagaaccgg aatatctctt 7440
tctgtactgt tcctataatt gtttcgttac ttccatatga aagtagattc atcaaggttt 7500
gattacataa tttattcact atttaattcc actcctacga atttatgtga tttttcaatt 7560
ctacaccact gttgctgtca aaatctgttt tcaagataaa ctttacctat tttgtggtca 7620
ccatggacca actaggattt tgccctacat aggtccacat gtgatcatat ttagccattc 7680
caatggctga tcattagccc aacacttcca tttcaaacca tagtcacatc atgaaaccat 7740
atatatacat acatacacac aaatggtcta atgccatact ccacttttac aagccatttt 7800
cgcatggctg tacacttata catttcataa agtactcgaa agacaacaat gggtagtcct 7860
atacatgcca tatcaaaatt caaccaaaat agtacccaaa agagcctttg aaagtggggg 7920
gcgacttcca ctttcaaatt cccaagtccg aatagctgga aaaccaaaaa tcttttaaaa 7980
aaaagaacca atgtaaacag taagccattt tttgcttata aagtttgagc aaagaaatcc 8040
cgccttaaaa aaaaaattcc aacaatttag ctaaagggaa aatttttata aaatcccatt 8100
ttttcgatat ttaaattttg tttccaaaaa ttaaaaaacc ctttgggtaa ataacccaaa 8160
aaaaataaat tataccaaaa gggcgggtac cctgttttat ctcctggggg ggaaaaaatt 8220
ttttttttta gagggcgtgc gatatatatt tttcaaacaa acacctatta taaaattctc 8280
cccccctaat tttgggtgga tggttttt 8308
SEQ ID NO: 106 (probe for contig 4198)
gacatgtttt gcccatcctt ccttgtcatg gccggccact actcattagg ggggggaatt 60
tgacatgcaa gtcccccctt tgtccacatg cactagtagg tcctcacaca ttgacctatc 120
acattttaga attttctcac ataagtccta ttgactaaat tcacatgaaa tcaaccaaat 180
tgaagcttga aattttcaca cattcataat tacatattct acacaataag tatcacattc 240
aaacatttcg gtgactcggt ttagcggtcc cgaaaccact tcccgactag ggtcaacttt 300
gggctgtcac aactctcccc cacttaagaa aattttcgtc cccgaaaatc ttaccggtaa 360
ataggtttgg gtatcgttct ttcatcgagc tctcggtctc ccaagtagct tcctcgatcc 420
cgtgtttgag ccataacacc tttactagcg gaactctttt gtttcgcaac tccttcactt 480
cacgagctag gatacgcatc ggttcttctt catagctcat gtcgtcttga atttcaacct 540
ctgatgggct aattatatgc gatggatcag atcgatagcg tcgaagcatc gagacatgaa 600
agacgtcatg aatcttttca agctccgggg gcaaaatcaa tctatacgca accgagccaa 660
ctcgctcgga gatttcgta 679
SEQ ID NO: 94 (contig 4199)
tgaaggcaga gagaaagaaa aaaaagagga ataaggctat agaggagaat atgaggtagg 60
agaagagggt aatggggttg agaaaaatag aaggggacac agagagatta gagaggcaaa 120
gagaagatag agctatggtg tgtagataag agttagaaaa taatattcga tgcatagcta 180
gttaaggata aagagattgg tactgagtgt taagttgagt gttttctcgg agagagttga 240
aaggaaaagc ggacctgcaa ctcttcgcac gcggccgcag ggaagaagag tataacagag 300
gtaaaataga aaataaaaaa tataaggggt aaaataaaat gatatcgaag ggatagcata 360
aaaaaggtct ccaaggtaaa gcgttaaaag ttatttttta atcgtcataa attactaggg 420
gcattagaag aggcgatagt cttaaatacc cattcaaaca attacaaatt taggtataaa 480
gtataatttt cggttcatcc cttttcccgt attggaattt gatgataatg gctttttaaa 540
acaattttag ggaatataat tgaaccatgt aattcatcaa gtctaggaat tggatgtcta 600
tactttactg ttattttgtt gattgggcgg caaacaacac acattctata cgtaccatct 660
ttcttcggta ccaataagac aggaacggca caatggctta ggctctcacg aatgtaccct 720
ttgtctagta actcctcaac ttgcctttgc aattcctttg tctcctcggg attgcttcta 780
taggctggtc gatttggcat tgaagctcct ggtactaagt caatctagtg ttctatccca 840
cgtaaaggtg gtaaaccttt aggggcctca ctaaaaacat cctcataatc ctgcaaaaga 900
cattgaacca cactaggcaa attttcatta atgttagata gagataaata gttttgccta 960
aacctcacaa ggatacaagg ctgtttattg agtagggctc tccgcacatc tttttttgtt 1020
gcaattaaaa ttttcactct agttttatca cttgcggatt cactcacctt tgattcattt 1080
aaaatttgac ttttttcact actagcatct tttctctctg tcttttttat ttgtcctttc 1140
ttcctcaccc cctcacaaaa tttcatcatt tttaattgat ctttatatac atcattggga 1200
tttaaaagag caaaagtgaa ttttcggcct ttaaacacaa gagagcttac ctttgtgtgt 1260
aacatcacga tcaaattgcc aaggccgtcc aaggagaaag tgtgccgcat gcattgatga 1320
ccacattaca ccatagatca tcctcgtaat tcttaagcta tatagtgagt gaggtactat 1380
tttgtaaact ttaactttcg agcaattcat aagccactga aggtgatacg gcttagggtg 1440
ctcgatacaa ggtaacttta aggaatccac caaatagctg ctaactacat tcgaacaact 1500
cccactatca ataataagtg aacataagtt accttttata aaacacccta gaatggaaaa 1560
tattggtcct ttggttctca aaatcgtctt tcatttggat gttaagggtg cggtttacta 1620
ccatcttatt ttttttcctc tttttttttg aaatacctgt aaaacaaata ctcaacactc 1680
aaaagaaaaa ttagcaaacc tcaccgttag tcactcaaaa agaaaaatca aattctcaat 1740
gaggtagaat tcagtcttgt gagttcttta gtagagtcta tatcaaccaa ttagaaagtg 1800
tatgaaccga aactaccaaa gagtcctaat ttgcactaag atgccaaaaa ctgacgagac 1860
accaattaat ttgtgttgca ccgaggaaga attgtgcaca ccttaaaatc ctactaacac 1920
aagaaacaag aaggtgttag atagtgtaaa ggaagaataa aggcaaacaa atgaaaacct 1980
acagctaaga atcaataaaa attgctgaaa tcgaaaaacc caaaaaactg cggagtaact 2040
tgacaacttc gttcgaggtg ttcccgatct ccaaaaatca caaaattaaa tctagaatgt 2100
ccttaaatat tgaatttttg atctggaaag tttgggcacc aaactcaacc cgctgaatct 2160
tttttaaaat ttttatttga tttcgtcttt ttcaattttt ttgacttttt cgaatgacgt 2220
ttttgcggga aatatttttt tatattcaat aacagtgcca aaaatatgta tgtaaaattt 2280
tagatcaatc agacaacgtt tacccactca aatgaatttt ttttgaacaa tttttctggg 2340
taaaactgct gtttgtgctt taaaaaatag agatcagttt agaaatcaac caggaacacc 2400
caaaacgccc aaaatctgat accaaatgat acgggggttg cgcgcggacc aagatcgagt 2460
tgccaagtca cgagaaactc ctaattattt taatatggaa attcggtcaa aggtcatttt 2520
atctgggact cttggactga atattgacat aaaaatttaa actaagtaaa taaataaata 2580
aataaagaaa aataaaaata aaactttaca acttgggcca ctttgacaat ttggcctgat 2640
ttttttaaca atttggcctg attttcaact aagtatggat ggatttcttg attgggctgg 2700
gaatttgctt attgggcctc gccttcaaga atttgggctt ttgtgactcg tatcattccc 2760
cccttcctca aaaagattcg tcctcgaatc tgaaaaatca aatgaactcg actttcctct 2820
atcgaacggg actaatggca attgagtcca ctgaaaagaa tagccatgag atagtaaata 2880
gcaacggaaa caagcttgta gtccaatcat aagcataaca gaacaggtat aacgcatgtg 2940
aatggacaga ttcagattac catgcaaaca atctaattta ctcaccactg cctcgtcaaa 3000
tatttgaaac aaagatggac atgttttaag gcattcagtc gtctcacatt gttcccacaa 3060
accatgaata aattgcgagt aataaatcaa atggtaaaca taatcgtcac aagtaggtat 3120
ttgaaaagat tcacatggtt cacagagttg cataatcata ccatgcatta atcgacggtc 3180
tatagattca ctcacacatg cattatcaaa aacaagaatc tttttatcaa ccatcaaaac 3240
agatagatca tcaataaata aaataggaca gtcaagcacg tttcgatcta agtgttcatc 3300
aacacgatct ttatcactat ccgaggagta caaaagtgtt tcaaaggttt caagcatctt 3360
acctcgataa gcagaagaat aagggtcgat tttacctttt tcatttaaaa caaaccttcg 3420
ctcatcgggg gcatagtgta gtcgaatctc cgttgttgag ttaacctttg tcaaatggaa 3480
ctcaaacttc atgtacaacc acataaactg tttaaggcac gaatataaat tgaaaacaaa 3540
tttggaaaat ttcgttacct tattctccaa aacagatttg gacaaattcg aggattttac 3600
acaaggtaaa tcaagagaat aacaaatcac gcttcttttc gtaatgactt tatcaaccac 3660
aatcgaagag taacaattaa tcggatcaaa atgaggggat cttttaagaa ctaagtcacc 3720
aaaagtttta tcaataattt tcaaatctgc actggactcg gtttctaaaa tttccttacc 3780
tcgcttacct tcgggatcat gtagtgtgat ttcatctttg cctaagttga tcgtatgaaa 3840
gcgtctttca tttgagaggt attgaagttg aaagttatct ttcgatcttt cgggaacctg 3900
aaaagtagca acacaagaaa aattcatatc ttggttagtg gaaacattcc tcactaccct 3960
ttccacgtct ttttcttttg tttctttttc taattcattt tctcttcttt cttcaatttc 4020
tttttcaatt ttcttttctg tttcacattc cttttcactc tcaatctctt tttgctcttt 4080
ttcattctct ttttcttttt cctcacttgt tttaacagaa tttttcagtt tgatttggtc 4140
ctcatggact tgttccggag tgagcggcac taaggtgagt ttctttcctt gatgcttgaa 4200
agaataccta ttggtacgac catcgtgagt gacttttcga tccagttgcc atggttctcc 4260
tagcaacaaa tggcaggctt gaataggcat tacgtcgcac acaacttcgt cttgatactt 4320
accgatagaa aaggcgatgc gcacttgttg agtgacccta aattggcctt cgttgctaag 4380
cccttgtagt tgataaggtt gtggatgctt ggtagtggtt aagcaaagct tttccaccat 4440
cgtcgtgctg gctatgttcg agcaactttc accatcaata ataactctac aaagcttacc 4500
ttgtacatgg cagcgagtat gaaagagatg ttctcgttgc tgctcgctct ttggttttgc 4560
caaagatgat tgtccacgat catgaaaatc atcatccatt ctagggacac gttgagggct 4620
gcacctatag ggctggttat ccctatcttc cttaattgga tctcgatatt gatgtttttg 4680
attcactcgt acctcattca aagtagtatt caatgcttga agtgtagcat ttatttgtgt 4740
gattgcagca gtatgttcgt ctaactgttg ttggactttc ataagtgcat cattattatc 4800
acctttagac atcttcaaaa cctgtaaaaa gtaaacactc aacactcaaa aataaaagtt 4860
agcaaacctc accattaatc actcaaaaag aaaaatcaaa ttctcaatga ggtcgaattc 4920
aatcttgtga gttctttatc agagtttata tcaaccaatt agagagtgtg aaccaaacta 4980
ccaaagaatc ctaatttgca ctaggatgcc aaaaactgac gagacaccaa gtgattcgtg 5040
ttgcaccgag gaaggagttg tgcacacgtg taaatccaac taacacaaga aacaagaagg 5100
tgttagataa cgtaacggaa gaataaaagc aaacaaatga aaacctacag ctaagaatca 5160
ataaaaattg ctgaaaatag aaaacccgaa aaactgcgga gtaacttgac aacttcgttc 5220
gaggtgttcc tgatctcaaa aaatcacgaa attaaatctt tgaatgttct tatatataga 5280
attttttatc tggaaatttt gggcaccaaa ttcaaccccg ctgaatcttt ttttaaattt 5340
ttaattgaat ttcatctttt ttttattttt ttggactttt ttgactaatt tttttggggg 5400
caataatttt tttttatttt caatccccag gggcccaaaa actgtgttgg taaaatattt 5460
tcaaaataaa tcgaaaaaac ttttaacccc cctccaaatg gaattttttt tttaaaaaaa 5520
tttttttttg gggaaaaaac tgtgctggtt ttttggtttt taaaaaaaaa ataaaaaatt 5580
tcgtttttaa aaaaatctaa ccccagaaaa aaccccccaa aaaacccccc aaaaaaattt 5640
taatacccca agggggtata acgggggacg ccccccgcgg gacaccccaa aaataacgca 5700
agttttttcc ccaagaatct ccccccggaa aaaaaaactc cccccttctc taaaaaaaaa 5760
aaaaaatttt tcttataaaa aaaaaaaa 5788
SEQ ID NO: 107 (probe for contig 4199)
tcaaatctgc actggactcg gtttctaaaa tttccttacc tcgcttacct tcgggatcat 60
gtagtgtgat ttcatctttg cctaagttga tcgtatgaaa gcgtctttca tttgagaggt 120
attgaagttg aaagttatct ttcgatcttt cgggaacctg aaaagtagca acacaagaaa 180
aattcatatc ttggttagtg gaaacattcc tcactaccct ttccacgtct ttttcttttg 240
tttctttttc taattcattt tctcttcttt cttcaatttc tttttcaatt ttcttttctg 300
tttcacattc cttttcactc tcaatctctt tttgctcttt ttcattctct ttttcttttt 360
cctcacttgt tttaacagaa tttttcagtt tgatttggtc ctcatggact tgttccggag 420
tgagcggcac taaggtgagt ttctttcctt gatgcttgaa agaataccta ttggtacgac 480
catcgtgagt gacttttcga tccagttgcc atggttctcc tagcaacaaa tggcaggctt 540
gaataggcat tacgtcgcac acaacttcgt cttgatactt accgatagaa aaggcgatgc 600
gcacttgttg agtgacccta aattggcc 628

### Example 5

### Characterization of G. hirsutum Centromeric DNA Structure

In these experiments, the investigators examined contig 4199 (SEQ ID NO:94) to discern structure t characteristic of the G. hirsutum centromere.

The investigators examined the sequence of the 4199 contig and found a sequenced BAC (GeneBank #EF457753) that contained sequence similar to contig 4199. By analysis of the BAC sequence, the investigators discovered the following elements, which are represented diagrammatically in FIG. 5A. As shown in FIG. 5A, the centromeric sequence comprises a CenCORE (represented by an oval) of approximately 2836 bp and two substantially identical flanking sequences, CenFR, each about 982 bp (represented by the arrows) (sequence analysis showed that each of CenFR (long terminal repeats (LTRs) was flanked by a TG/CA inverted repeat at each end). The CenCORE sequence is represented by SEQ ID NO:95; and the CenFR sequence by SEQ ID NO:96. SEQ ID NOs:95, 96 and 97 (the latter representing the sequence of the whole construct, designated “CRG1” and sometimes referred to as “CEN”) are shown in Table 8.

TABLE 8
Consensus sequences for CenCORE (SEQ ID NO: 95), CenFR (SEQ ID NO: 96)
and CRG1 (SEQ ID NO: 97) of G. hirsutum

SEQ ID NO: 95 (CenCORE)
ttcccccctt cctcaaaaag attcgtcctc gaatctgaaa aatcaaatga actcgacttt 60
cctctatcga acgggactaa tggcaattga gtccactgaa aagaatagcc atgagatagt 120
aaatagcaac ggaaacaagc ttgtagtcca atcataagca taacagaaca ggtataacgc 180
atgtgaatgg acagattcag attaccatgc aaacaatcta atttactcac cactgcctcg 240
tcaaatattt gaaacaaaga tggacatgtt ttaaggcatt cagtcgtctc acattgttcc 300
cacaaaccat gaataaattg cgagtaataa atcaaatggt aaacataatc gtcacaagta 360
ggtatttgaa aagatttaca tggttcacag agttgcataa tcataccatg cattaatcga 420
cggtctatag attcactcac acatgcatta tcaaaaacaa gaatcttttt atcaaccatc 480
aaaacagata gatcatcaat aaataaaata ggacagtcaa gcatgtttcg atctaagtgt 540
tcatcaacac gatctttatc actatccgag gagtacaaaa gtgtttcaaa ggtttcaagc 600
atcttacctc gataagcaaa agaataaggg tcgattttac ctttttcatt tgaaacaaac 660
cttcgttcat cgggggcata gtgtagtcga atctccgttg ttgagttaac ctttgtcaaa 720
tggaactcaa acttcatgta caaccacata aactgtttaa ggcaagaata taaattgaaa 780
acaaatttgg caaatttcgt taccttattc tccaaaacag atttggacaa attcaaggat 840
tttacacaag gtaaatcaag agaataacaa atcacgcttc ttttcgtaat gactttatca 900
accacaatcg aagagtaaca attaatcgga tcaaaatgat gggatctttt aagaactaag 960
tcaccaaaag ttttatcaat aattttcaaa tctgcactgg actcggtttc taaaatttcc 1020
ttacctcgct taccttcggg atcatgtagt gtgatttcat ctttgcttaa gttgatcgta 1080
tgaaagcgtc tttcatttga gaggtattga agttgaaagt tatctttcga tctttcggga 1140
acctgaaaag tagcaacaca agaaaaattc atatcttggt tagtggaaac attcctcact 1200
agcctttcct cgtctttttc ttttgtttct ttttctaact cattttctct tctttcttca 1260
acttcttttt caattttctt ttctgtttca cattcctttt cactctcaat ctctttttgc 1320
tctttttcat tctctttttc tttttcctca cttgttttaa cagaatttct cagtttgatt 1380
tggtcctcat ggacttgttc cggagtgagc ggcactaagg taagtttctt tccttgatgc 1440
ttgaaagaat acctattggt acgaccatcg tgagtgactt ttcgatccag ttgccatggt 1500
tctcctagca acaaatggca ggcttgaata ggcattacgt cgcacacaac ttcgtcttga 1560
tacttaccga tagaaaaggc aatgcgcact tgttgagtga ccctaaattg gccttcgttg 1620
ctaagccctt gtagttgata aggttgtgga tgcttggtag tggttaagca aagcttttcc 1680
accatcgtcg tgctggctat gttcgagcaa ctttcaccat caataataac tctacaaagc 1740
ttaccttgta catggcagcg agtatgaaag agatgttctc gttgctgctc gctccttggt 1800
tttgccaaag atgattgtcc acgatcatga aaatcatcat ccattctagt gacacgtcga 1860
gggccgcgcc tttggggttg gttatcccta tcttccttaa ttggatctcg atattgatgt 1920
ttttgattca ctcgtatctc attcaaagta gtattcaatg cttgaagtgt agcatttatt 1980
tgtgtgattg cagcagtatg tccatctaac tgttgttgga ctttcataag tgcatcatta 2040
ttatcacctt tagacatctt caaaacctgt aaaaaataaa cactcaacac tcaaaaataa 2100
aagttagcaa acctcaccat taatcactca aaaagaaaat tcaaattctc aatgaggtcg 2160
aattcaatct tgtgagttct ttatcagagt ttatatcaac caattagaga gtgtgaacca 2220
aactaccaaa gaatcctaat ttgcactatg atgccaaaaa ctgacgagac accaattgat 2280
tcgtgttgca ccgaggaaga agttgtgcac acgtttaaat cctactaaca caagaaacaa 2340
gaaggtgtta gataacgtaa aggaagaata aaggtaaaca aatgaaaacc tacagctaag 2400
aatcaataaa aattgctgaa aacagaaaac ccgaaaaact gcggagtaac ttgacaactt 2460
cgttcgaggt gttcccgatc tccaaaaatc acgaaattaa atctggagtg tccttatata 2520
tttaattttt gatctggaaa atttgggcac caaattcaac ccgctgaata tttttttaat 2580
tttttattgg atttcgtctt ttttcaattt tttgactttt tcgacttatt tttttgcggg 2640
aaatattttt gtatattcaa taacagtgcc aaaaatatgt atgtaaaatt tcagatcaat 2700
cagaaaacgt ttacccactc aaataaattt ttttcgaaaa tttttctggg taaaactgct 2760
gtttgtaatt taaaaaatag agatcagttt agaaatcaac caagaacacc caaaacgccc 2820
aaaatctgat accaaa 2836
SEQ ID NO: 96 (CenFR)
tgatacgggg ttgcgcgcgg accaagatcg agttgccaag tcacgggaaa ctcctacgaa 60
aacccttaaa caattagatc tgaaacgaaa cagaaaatta aaagattaga tttttgaatt 120
ttcagatctg aaataaaatc cccaaatcag caaagaatca aattgagaat agaaataagt 180
gttagggttc ttgaaaccct caaggagatt gtgattctgc ccaattgaac actaagatag 240
ttttccccaa atttcgacaa tctaatttca cccaaaaaga gtatggaaaa accctagaaa 300
ttggggattt ttgggctgat tccttaagat agaaaaaggc tgaaaacaca ataagaacag 360
aaaatagatt agataatgat tcagcacaag tagaaataaa gaaagaattg acagtaagaa 420
attaaaagat aagtcctaag aagccttgaa atctcgaaag atctcacaac tcccttcaaa 480
cggctctaat ctcccctcca aagaatatca atggcaagaa gaaggttgaa gatggctccc 540
acaatcaaaa agattgttaa aacaacttct aaagaaaact caagagaaaa tccttgaaga 600
actcaaagag aattttcact caaatcaaat ctgaaatttt caatgtaatt gtaatgtaat 660
gtagggtggc tggccaagcc atataaatag gcctttcaaa tgttttccta atttaattag 720
aacactaaaa ctaaaactaa aaaaaactcc taattatttt aatatggaaa ttcggccaag 780
ggtcttttat ttgggactct tggactgaat ataaaaattt aaactaaata aaataaataa 840
ataaaaataa aactttacaa cttgggccac tttgataatt tggcctgatt ttcaactaag 900
tatgggtgga tttcttgatt gggctgagaa tttgcttatt gggcctcgcc ttcaagaatt 960
tgggcttttg tgactcgtat ca 982
SEQ ID NO: 97 (CRL1)
tgaaggcaga gagaaagaaa aaaaagagga ataaggctat agaggagaat atgaggtagg 60
agaagagggt aatggggttg agaaaaatag aaggggacac agagagatta gagaggcaaa 120
gagaagatag agctatggtg tgtagataag agttagaaaa taatattcga tgcatagcta 180
gttaaggata aagagattgg tactgagtgt taagttgagt gttttctcgg agagagttga 240
aaggaaaagc ggacctgcaa ctcttcgcac gcggccgcag ggaagaagag tataacagag 300
gtaaaataga aaataaaaaa tataaggggt aaaataaaat gatatcgaag ggatagcata 360
aaaaaggtct ccaaggtaaa gcgttaaaag ttatttttta atcgtcataa attactaggg 420
gcattagaag aggcgatagt cttaaatacc cattcaaaca attacaaatt taggtataaa 480
gtataatttt cggttcatcc cttttcccgt attggaattt gatgataatg gctttttaaa 540
acaattttag ggaatataat tgaaccatgt aattcatcaa gtctaggaat tggatgtcta 600
tactttactg ttattttgtt gattgggcgg caaacaacac acattctata cgtaccatct 660
ttcttcggta ccaataagac aggaacggca caatggctta ggctctcacg aatgtaccct 720
ttgtctagta actcctcaac ttgcctttgc aattcctttg tctcctcggg attgcttcta 780
taggctggtc gatttggcat tgaagctcct ggtactaagt caatctagtg ttctatccca 840
cgtaaaggtg gtaaaccttt aggggcctca ctaaaaacat cctcataatc ctgcaaaaga 900
cattgaacca cactaggcaa attttcatta atgttagata gagataaata gttttgccta 960
aacctcacaa ggatacaagg ctgtttattg agtagggctc tccgcacatc tttttttgtt 1020
gcaattaaaa ttttcactct agttttatca cttgcggatt cactcacctt tgattcattt 1080
aaaatttgac ttttttcact actagcatct tttctctctg tcttttttat ttgtcctttc 1140
ttcctcaccc cctcacaaaa tttcatcatt tttaattgat ctttatatac atcattggga 1200
tttaaaagag caaaagtgaa ttttcggcct ttaaacacaa gagagcttac ctttgtgtgt 1260
aacatcacga tcaaattgcc aaggccgtcc aaggagaaag tgtgccgcat gcattgatga 1320
ccacattaca ccatagatca tcctcgtaat tcttaagcta tatagtgagt gaggtactat 1380
tttgtaaact ttaactttcg agcaattcat aagccactga aggtgatacg gcttagggtg 1440
ctcgatacaa ggtaacttta aggaatccac caaatagctg ctaactacat tcgaacaact 1500
cccactatca ataataagtg aacataagtt accttttata aaacacccta gaatggaaaa 1560
tattggtcct ttggttctca aaatcgtctt tcatttggat gttaagggtg cggtttacta 1620
ccatcttatt ttttttcctc tttttttttg aaatacctgt aaaacaaata ctcaacactc 1680
aaaagaaaaa ttagcaaacc tcaccgttag tcactcaaaa agaaaaatca aattctcaat 1740
gaggtagaat tcagtcttgt gagttcttta gtagagtcta tatcaaccaa ttagaaagtg 1800
tatgaaccga aactaccaaa gagtcctaat ttgcactaag atgccaaaaa ctgacgagac 1860
accaattaat ttgtgttgca ccgaggaaga attgtgcaca ccttaaaatc ctactaacac 1920
aagaaacaag aaggtgttag atagtgtaaa ggaagaataa aggcaaacaa atgaaaacct 1980
acagctaaga atcaataaaa attgctgaaa tcgaaaaacc caaaaaactg cggagtaact 2040
tgacaacttc gttcgaggtg ttcccgatct ccaaaaatca caaaattaaa tctagaatgt 2100
ccttaaatat tgaatttttg atctggaaag tttgggcacc aaactcaacc cgctgaatct 2160
tttttaaaat ttttatttga tttcgtcttt ttcaattttt ttgacttttt cgaatgacgt 2220
ttttgcggga aatatttttt tatattcaat aacagtgcca aaaatatgta tgtaaaattt 2280
tagatcaatc agacaacgtt tacccactca aatgaatttt ttttgaacaa tttttctggg 2340
taaaactgct gtttgtgctt taaaaaatag agatcagttt agaaatcaac caggaacacc 2400
caaaacgccc aaaatctgat accaaatgat acgggggttg cgcgcggacc aagatcgagt 2460
tgccaagtca cgagaaactc ctaattattt taatatggaa attcggtcaa aggtcatttt 2520
atctgggact cttggactga atattgacat aaaaatttaa actaagtaaa taaataaata 2580
aataaagaaa aataaaaata aaactttaca acttgggcca ctttgacaat ttggcctgat 2640
ttttttaaca atttggcctg attttcaact aagtatggat ggatttcttg attgggctgg 2700
gaatttgctt attgggcctc gccttcaaga atttgggctt ttgtgactcg tatcattccc 2760
cccttcctca aaaagattcg tcctcgaatc tgaaaaatca aatgaactcg actttcctct 2820
atcgaacggg actaatggca attgagtcca ctgaaaagaa tagccatgag atagtaaata 2880
gcaacggaaa caagcttgta gtccaatcat aagcataaca gaacaggtat aacgcatgtg 2940
aatggacaga ttcagattac catgcaaaca atctaattta ctcaccactg cctcgtcaaa 3000
tatttgaaac aaagatggac atgttttaag gcattcagtc gtctcacatt gttcccacaa 3060
accatgaata aattgcgagt aataaatcaa atggtaaaca taatcgtcac aagtaggtat 3120
ttgaaaagat tcacatggtt cacagagttg cataatcata ccatgcatta atcgacggtc 3180
tatagattca ctcacacatg cattatcaaa aacaagaatc tttttatcaa ccatcaaaac 3240
agatagatca tcaataaata aaataggaca gtcaagcacg tttcgatcta agtgttcatc 3300
aacacgatct ttatcactat ccgaggagta caaaagtgtt tcaaaggttt caagcatctt 3360
acctcgataa gcagaagaat aagggtcgat tttacctttt tcatttaaaa caaaccttcg 3420
ctcatcgggg gcatagtgta gtcgaatctc cgttgttgag ttaacctttg tcaaatggaa 3480
ctcaaacttc atgtacaacc acataaactg tttaaggcac gaatataaat tgaaaacaaa 3540
tttggaaaat ttcgttacct tattctccaa aacagatttg gacaaattcg aggattttac 3600
acaaggtaaa tcaagagaat aacaaatcac gcttcttttc gtaatgactt tatcaaccac 3660
aatcgaagag taacaattaa tcggatcaaa atgaggggat cttttaagaa ctaagtcacc 3720
aaaagtttta tcaataattt tcaaatctgc actggactcg gtttctaaaa tttccttacc 3780
tcgcttacct tcgggatcat gtagtgtgat ttcatctttg cctaagttga tcgtatgaaa 3840
gcgtctttca tttgagaggt attgaagttg aaagttatct ttcgatcttt cgggaacctg 3900
aaaagtagca acacaagaaa aattcatatc ttggttagtg gaaacattcc tcactaccct 3960
ttccacgtct ttttcttttg tttctttttc taattcattt tctcttcttt cttcaatttc 4020
tttttcaatt ttcttttctg tttcacattc cttttcactc tcaatctctt tttgctcttt 4080
ttcattctct ttttcttttt cctcacttgt tttaacagaa tttttcagtt tgatttggtc 4140
ctcatggact tgttccggag tgagcggcac taaggtgagt ttctttcctt gatgcttgaa 4200
agaataccta ttggtacgac catcgtgagt gacttttcga tccagttgcc atggttctcc 4260
tagcaacaaa tggcaggctt gaataggcat tacgtcgcac acaacttcgt cttgatactt 4320
accgatagaa aaggcgatgc gcacttgttg agtgacccta aattggcctt cgttgctaag 4380
cccttgtagt tgataaggtt gtggatgctt ggtagtggtt aagcaaagct tttccaccat 4440
cgtcgtgctg gctatgttcg agcaactttc accatcaata ataactctac aaagcttacc 4500
ttgtacatgg cagcgagtat gaaagagatg ttctcgttgc tgctcgctct ttggttttgc 4560
caaagatgat tgtccacgat catgaaaatc atcatccatt ctagggacac gttgagggct 4620
gcacctatag ggctggttat ccctatcttc cttaattgga tctcgatatt gatgtttttg 4680
attcactcgt acctcattca aagtagtatt caatgcttga agtgtagcat ttatttgtgt 4740
gattgcagca gtatgttcgt ctaactgttg ttggactttc ataagtgcat cattattatc 4800
acctttagac atcttcaaaa cctgtaaaaa gtaaacactc aacactcaaa aataaaagtt 4860
agcaaacctc accattaatc actcaaaaag aaaaatcaaa ttctcaatga ggtcgaattc 4920
aatcttgtga gttctttatc agagtttata tcaaccaatt agagagtgtg aaccaaacta 4980
ccaaagaatc ctaatttgca ctaggatgcc aaaaactgac gagacaccaa gtgattcgtg 5040
ttgcaccgag gaaggagttg tgcacacgtg taaatccaac taacacaaga aacaagaagg 5100
tgttagataa cgtaacggaa gaataaaagc aaacaaatga aaacctacag ctaagaatca 5160
ataaaaattg ctgaaaatag aaaacccgaa aaactgcgga gtaacttgac aacttcgttc 5220
gaggtgttcc tgatctcaaa aaatcacgaa attaaatctt tgaatgttct tatatataga 5280
attttttatc tggaaatttt gggcaccaaa ttcaaccccg ctgaatcttt ttttaaattt 5340
ttaattgaat ttcatctttt ttttattttt ttggactttt ttgactaatt tttttggggg 5400
caataatttt tttttatttt caatccccag gggcccaaaa actgtgttgg taaaatattt 5460
tcaaaataaa tcgaaaaaac ttttaacccc cctccaaatg gaattttttt tttaaaaaaa 5520
tttttttttg gggaaaaaac tgtgctggtt ttttggtttt taaaaaaaaa ataaaaaatt 5580
tcgtttttaa aaaaatctaa ccccagaaaa aaccccccaa aaaacccccc aaaaaaattt 5640
taatacccca agggggtata acgggggacg ccccccgcgg gacaccccaa aaataacgca 5700
agttttttcc ccaagaatct ccccccggaa aaaaaaactc cccccttctc taaaaaaaaa 5760
aaaaaatttt tcttataaaa aaaaaaaa 5788

To determine if this structure correlated with centromeric localization, the investigators prepared these sequences (SEQ ID NOs:95 and 96) as probes for FISH analysis. Cotton mitotic metaphase chromosomal preparations were probed. Chromosomes were visualized by counter-staining with DAPI.

FIGS. 5B-51 shows the results of these experiments. When metaphase chromosomes are probed with SEQ ID NO:95, specific to the CenCORE, signal is observed on every chromosome and localizes to the centromeric constrictions (FIG. 5B; a magnified view of one of the labeled chromosomes is shown in FIG. 5F). Similar results are observed when the same preparations are probed with SEQ ID NO:96, specific to the CenFR sequences (FIG. 5C; a magnified view, FIG. 5G). When the signal from the two probes, SEQ ID NOs:95 and 96, are overlain, they co-localize (FIG. 5E; a magnified view, FIG. 5I), indicating that the two sequences are centromeric. FIG. 5D shows the same preparation stained with DAPI (magnified view, FIG. 5H).

The investigators observed that each CenFR was 100% identical (SEQ ID NO:96), indicating that this element inserted recently (SanMiguel, Gaut et al. 1998). The investigators search Gossypium genomic sequence using CenFR (SEQ ID NO:96), identifying a 10,911 nucleotide element was designated CRG2, with high sequence similarity to CRG1 (95% identity of the LTRs). Also, the two LTRs of CRG2 were observed to be only 92% identical. The investigators also queried the publicly avaialbe cotton expression sequence databases to determine if any other sequence aligned with the CenFR and CenCORE region. Interestingly, they observed several expressed sequence tags (ESTs), and an internal region of ATGP5, a gypsy-like LTR retrotransposon. Functional centromere-specific retrotransposon elements have been described in monocots (e.g., maize and rice), but not in dictots. The investigators next analyzed the relationship between CRG and other known retroelements, including centromere retroelements from other species. BLAST searches against the nonredundant nucleotide collection showed that the polyproteins of both CRG1 and CRG2 show the highest sequence similarity to Ty3-gypsy elements.

The identification of a higher plant centromere that does not comprise an array of tandem repeat sequences is unexpected and thus surprising and novel.

### Example 6

### Genome Specificity of Centromeric DNA

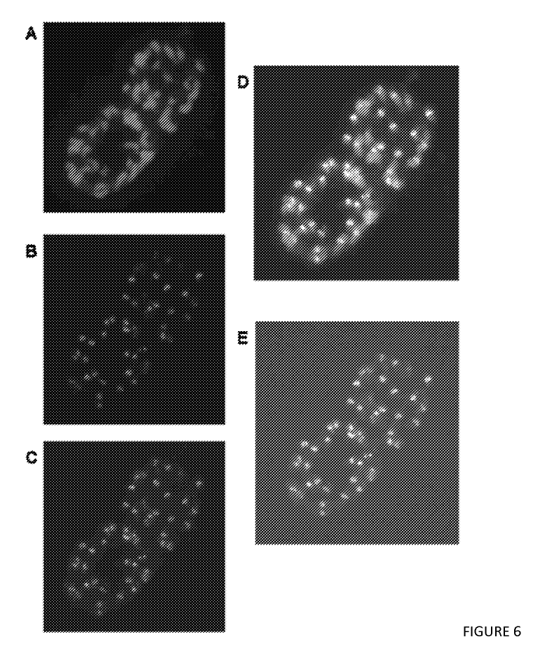

By probing the BAC library with contig4199 (SEQ ID NO:94) using standard molecular biology techniques, BACs containing centromeric sequences were identified. Fifty-four BACs were chosen for further characterization. The investigators used primers to amplify CenCORE (SEQ ID NO:95) and CRG1 (SEQ ID NO:97), having both a CenCORE (SEQ ID NO:95) and the CenFL (SEQ ID NO:96) sequences, from individual BACs, shown in Table 9. During analysis of the amplified sequences, the investigators observed that instead of the expected approximately 3 kb CenCORE fragment, an about 6 kb fragment was amplified from 3 of the 54 BACs. Using this 6 kb PCR fragment as a probe for FISH analysis as described in Example 2, half of the centromeres showed stronger signal than others, as shown in FIG. 6. FIG. 6A shows the sample counterstained with DAPI. FIG. 6B shows staining with 6 kb CenCORE probes (amplified by the primers of SEQ ID NOs:109 and 110), showing (1) every chromosome being labeled at the centromeric constriction, as expected, and (2) a sub-population of chromosomes showing qualitatively significantly stronger signal than other chromosomes. FIG. 6C shows the same sample probed for the CRG1 (SEQ ID NO:96) sequence, showing a more even distribution of signal. When the images are overlaid (FIG. 6D, showing the merged images of FIGS. 6A-C; and FIG. 6E, showing the merged images of FIGS. 6B and 6C), the signals are observed to overlap, and the difference in staining intensity of a subpopulation of chromosomes is again observed. These results indicate that the 6 kb fragment probe amplified by SEQ ID NOS:109 and 110 binds to a sub-genome specific centromere sequence.

G. hirsutum is a tetraploid derived from a recent allopolyloidization event, which brought together a New World D genome and an African-Asian A genome approximately a million years ago (Wendel and Cronn 2002). To determine whether the CRG element was present in the centromere regions of the progenitor diploid Gossypium A and D genomes, the investigators used dot blot hybridization and FISH to verify CRG presence and localization in existing A and D diploid species. The investigators found that the CRG element was observed in the three D genome species examined (G. davidsonii D3-3, G. klotzschianum D3-K, G. raimondii D5-2; FIG. 4). Sequences highly similar to the CRG element were also observed after analyzing BLAST searches using the CRG element as a basis for comparison in the G. raimondii D genome genomic survey sequences, confirming the dot blot results. In these species, the CRG element also localizes to the centromere region. However, no hybridization was detected, either by dot blot or by FISH in the A genome species tested (G. herbaceum A1-5, G. arboreum, A2).

To determine how widespread the occurrence of the CRG1 or CRG2 element is in other Gossypium genomes, the investigators examined other diploid cotton species by the same methods. Although the CRG element was not present in the A genome species tested, the investigators observed it in the centromere regions of two other African-Asian species, the B genome G. anomalum and the E2 genome G. somalense, but not in the E1 genome G. stocksii. The F genome African-Asian species, G. longicalyx, was also negative, as were three Australian species tested, G. nandewarense, Cl, G. puichellum C8-1, and G. nelsonii, G. These results are summarized in Table 10. Thus, the CRG element appears to be present in both New World and Old World lineages of diploid cottons, but absent in Australian and some Old World lineages.

TABLE 9
Primers for amplifying CenCORE (SEQ ID NO: 95)
and CRG1 (SEQ ID NO: 97) sequences

| SEQ | | |
|---|---|---|
| ID NO | Sequence | Target |
| 109 | cacgggaaac tcctacgaaa | SEQ ID NO: 95 Forward |
| 110 | cgagtcacaa aagcccaaat | SEQ ID NO: 95 Reverse |
| 111 | cccccttcct caaaaagatt | SEQ ID NO: 96 Forward |
| 112 | atcagatttt gggcgttttg | SEQ ID NO: 96 Reverse |

TABLE 10
Summary of CRG element analyses in different cotton species

| | | CRG1 | CRG2 | CRG | CRG BAC | Gh1 | pXP | 5S | 18S |
|---|---|---|---|---|---|---|---|---|---|
| Species | | internal | internal | LTR | 53H10 | (194bp) | 1-80 | rDNA | rDNA |
| G. herbaceum | A1-5 | − | − | − | − | − | + | + | + |
| G. arboreum | A2 | − | − | − | − | − | + | + | + |
| G. anomalum | B1 | + | + | + | +/− | − | ++ | + | + |
| G. pulchellum | C8-1 | − | − | − | − | − | + | + | + |
| G. nandewarense | C1 | − | − | − | − | − | + | +/− | + |
| G. davidsonii | D3-3 | + | + | + | + | + | + | + | + |
| G. klotzschianum | D3-K | + | + | + | + | + | + | + | + |
| G. ramondii | D5-2 | +/− | +/− | +/− | +/− | +/− | ++ | +/− | ++ |
| G. hirsutum | AD1 | + | + | + | + | + | + | + | + |
| G. barbadense | AD2 | + | + | + | +/− | + | + | + | + |
| G. mustelinum | AD4 | + | + | + | + | +/− | + | + | + |
| G. hirsutum | AD1 | + | + | + | + | + | ++ | + | ++ |
| TM-1 | | | | | | | | | |
| G. stocksii | E1 | − | − | − | − | − | + | + | + |
| G. somalense | E2 | + | + | +/− | +/− | + | ++ | + | ++ |
| G. longicalyx | F1 | − | − | − | − | − | + | + | + |
| G. nelsonii | G | − | − | − | − | − | + | + | + |

### Example 7

### Additional Exemplary G. hirsutum Centromere Sequences

To identify additional centromere sequences from G. hirsutum, the investigators used two approaches: a bioinformatics approach, and a BAC library probing approach. In the first approach, BLAST searches were run, using CRG1 (SEQ ID NO:97) [is this correct?] as a basis for comparison. The BLAST parameters were NCBI BLAST default parameters From these studies, 32 additional GenBank accessions were identified having similar structures (CenFR-CenCORE-CenFR); from these, four were chosen for further study and there corresponding CEN sequences are shown in Table 11.

TABLE 11
Exemplary CEN sequences (SEQ ID NOs: 113, 116, 119, and 122)

SEQ ID NO: 113 (from GenBank Ac187405)
gcccatagtg ggggtttaat gggacatttt ggagtggcca aaacactaga tatcttgcaa 60
gaacactttc attggccaca tatgaagaag gatgtggaaa aggtatgttc caagtgcatt 120
acatgcaaac aagcaaaatc taaggtaatg cctcatggcc tttacactcc tctaccgatt 180
cctacttcac cttgggtaga tttatccatg gattttattt taggttttcc tcgaactaag 240
aaaggaagag atagtatatt tgttgttgtt gacaggtttt caaagatgac acattttgtt 300
tcttgtcaca aaacagatga tgctacacat gtggcagact tattctttaa agaggtggta 360
agacttcatg gcatccctaa aacaattgtt tctgatacag atgtcaaatt ccttagccac 420
ttttggaaga tattgtgggg taagcttggt actaaattac tttattctac tacatgtcac 480
ccccaaactg atggtcaaac tgaagtagtt aatcgagtct tagggacttt attacgagct 540
gttgtgggaa aggacattag aaattgggaa gaatgcctac catttgttga atttgcatat 600
aatagatcta ttcattctac aactggttat tctccatttg aacttgttta tggctttaac 660
ccactaacag tacttgatct tgcgccactc cctcttgaac acattataaa tttagatggc 720
gaatagaaag ctgagttggt gaaatcccta catgagaagg ctaggcaacg aatagccaaa 780
acaaatgatg taaacaccaa caaagcaaac aaggg 815
SEQ ID NO: 116 (from GenBank Ac188141)
ttcgacatgg gatgagttga aacagattat gcgtaaaagg tttattccac ctcactacta 60
tagagaaatt aaaacaaagt tgagaagact tatccaaggt agtaggacgg tggatgaata 120
ttttaaagag atggagatgc tcattcagag agctaatgtg gaggaagatg aagaaacaac 180
tatggttcga ttcattgatg gtttgaacag gccgatagct aatacattaa ggctacaaac 240
ttacattgat ttggaagaag cagttcacaa agccattgag atcgagcaac aattaaagga 300
acaaaggttt gggtccttct ctacttcaca atattatcga ggtaacaatt ccaattctga 360
ttttaagagt tcaaaaccac cttttgttac taacaagtct tctttgagta atggaagtaa 420
acaatcagat tggaagaaag gggctcctgc taaaatgcaa acaccttcta agcagcccaa 480
tttaggtgat tcgagtgcga agaggactcg tgagattgaa tgctttaagt gcaaagggcg 540
tggtcattat agtagagaat gccctaacac gagattactt cttctaaaag ataacggtga 600
gtacacgtcc gactctgata atacagatcc agatatgcca gaacttgtgg atgacagtga 660
taatggcgac gagttggtca aaccaccaaa ggaaggggac tttgctaact ttcaatgcct 720
tgtagttcgt cgaaccctta acattcagat gaaagatgat gatagccaaa gggccaatat 780
tttccattct cggtgtttta ttaaaggtaa cttatgttca ctcattattg atagtggtag 840
ttgctcaaat gtggtgagta gctatttggt tgattcttta aagttacctt gcaccaaaca 900
ccctaagcca tatcaccttc agtggcttaa tgaatgttcc gaagttaaag ttacgaaaca 960
gtccttggtc acttttaaac tcggcaatta tgaggatgaa gtatggtgtg atgtagtccc 1020
aatgcatgcg gcacacttgc tccttggacg accttggcaa tttgat 1066
SEQ ID NO: 119 (from GenBank Ac193955)
tggttggtca catcaaggaa gcttggacca actcaaaaag caatccgtgc actttaatac 60
gagctgaatt aacttccatt tcagctcaat gagctcgaat tagctcaact tcagctcaaa 120
ccttttcaat tcagcttaat atcaactaga cattaattta atagctgaat atttatttaa 180
gtcgatttaa ttagtttatt tcagcttatt aatatgtttg ttgcatgttt ttaatatgtc 240
aagttttatt acatgatttt aagttgcttt aaagctgccg aatttaatga gctgatttta 300
gtgatatagt tgaccgaatt aatgtttcat ggttttaata ttaattttgt taagctaaat 360
atcatattaa ttttagttct tcttatttat ataggttggc cgaatgtgtg ggatcattta 420
acaaggttag ccgaatttga agattcattt tagcaaggtg catgcatgtt agatacattg 480
cacctggaga tacatgcatc aaattggtga gctgaattca acattcctag gtgactattc 540
ggccaaggga gctaatgttt ccaactccaa aactgcccat taattccttt cacatttgct 600
ggatgcatct tggcttttcg gttccatgtg ttcacactcc atgactcttc aacgttgagt 660
tttcacactt gaattggctg ctcattatcc tcttaattgc tggtcacctt tcagccaaaa 720
gttacaacat aaatgagcta atttatgccc attggccgaa cctagctatc catgcatcat 780
ctaagttttt ctaagtccaa tgcttcatct agaaggtgtc taatggagct ccatgttcag 840
ccgaatctag ctagttcatt taagtgattc atttgcttaa tttttaagga atgtaagtga 900
ccattcggct agcttaggtg ttgcttataa atagctgaaa ttttactttg taaagaactt 960
ttgacatttt taattagcga attgctgcca aaattgtgag gcattttctc tcaaatttcg 1020
ttcgagaccc gtaagactta tctaagcttt tagtggcatc tttccgagtc ttttccgaac 1080
ttatcactcc ttaaacccga gtgtggcgtt cacctttgat acctttggtt catactttcc 1140
taagtatcgg gtcaaggtcc ttttcaacca tttccaattc atttaggtcc tataagtttc 1200
gggtcgacac ttttctttag tgttggttca ttcttgacct ctttgaacca ttaccaattc 1260
gtaccaaata ccataccatt tcataccaat tccataccat ttcgtaccaa ttcccaaata 1320
ccaaaacgta gcaaaaccaa ataccatttc ttaattatct taaaccgaac tcttccattc 1380
tattcatacc atttcgactt aaaccaaatc cacatccaac tttgcacaaa cttatcttac 1440
ttctttgtac acaaatatat tctaactcct cgtctaatct acttcttcgt ttacgatcgg 1500
gaaccaaacg actcggactc 1520
SEQ ID NO: 122 (from GenBank Ac197186)
gtaaaccgcc atcggaatta tgaaggtgag atttcgacat gggatgagtt gaaacagatt 60
atgtgtaaaa ggtttattcc acctcactat tatagagaaa ttaaaacaaa gctgagaaga 120
cttatccaag gtagtaggac ggtggatgaa tattttaaag agatggagat gctcattcag 180
agagctaaca tggaggagga tgaagaaaca actatggttc gattcattga tggtttgaac 240
aggccgatag ctaatacatt gaggctacaa acttatattg atttggaaga agcagttcac 300
aaagccattg agatcgagca acaattaaag gaacaaaggt ttgggtcatt ctctacttca 360
caatattacc gaggtaacaa ttccaattct gattttaaga gttcaaaacc accttttgtt 420
actaataagt cttctttgag taacaagcag ttagattgga aaaaaggggc tcctgctaaa 480
acgcaaacac cttctaagta gcccaattta ggtgattcga gtgtgaagag gactcgtgag 540
gttgaatgtt ttaagtgcaa agggcgtggt cattatagta gggaatgccc taacacgaga 600
ttccttcttc taaaagataa tggtgagtat acttccgact ctgataaaac agatccagat 660
atgccagaac ttgttgatga cagtgataat ggcgaagagt tggttgaacc actcagagaa 720
ggagactttg ctaattttca atgccttgta gttcgtcgaa cccttaacat tcagatgaaa 780
gatgatgata gccaaagggc caatattttc cattctcggt gttttattaa aggtaactta 840
ttgatgtgat ccggctcggt tgattgagtc gattcacttg attgcccgat cgtcgacgag 900
aagtttgttc gattttgtag atagatggag ttagatgatt tttttatagc ggaagctgag 960
aaaatgggtt caaaagatgg tttggataga tgtttgatag gttcggtgaa ataaggaaat 1020
attgtttata atttaatagt taaaatggaa tggaaaaata gaactcaagc gaagaacgat 1080
tcaatactat atgaagtatt gccccgggac ttatattgct caaggtggat tatatggatt 1140
gtcgaagatg gaccttgacc cgttacctat agagaggtag gaatcaaagg tttaaaaagg 1200
tggatgaacg ccacaccagg atggtgataa gttcaagaag tcgggttgac gccactagcg 1260
agctagataa gtcagacgag actcaaacga aatcaggttg gaggtaacct cacaataagt 1320
ttaataatca gaaatgtctg taacctttta caataaacaa gtaagctatt tataagccta 1380
aaatgcctat tgatattcgg catctatgtt gttcacatta acttaaatcc tgttcacata 1440
ggtatttaac atgttcacac aaaaagcatt aaaattcggt tcatgcttga aaatggtaca 1500
tgaattggcc gaaccatcat gatgcttcac ttgatgcatt catgcatcct tagccttgaa 1560
ggatctccat aattccgtga atgtgcagcc ctttaatgat cctacatctc ccctggctga 1620
atgaggcatt aatctgccta aaatacttga atggtcatct tgaaaatgca tgaatcatgc 1680
atgaaacgtc ccatgtgttt tcccccataa atatgatcca tgaatcttca aatacatgaa 1740
ctttcagcaa ctccctcttg catgaacgtc ccaaatgcat gtgctccttt aaatgccatc 1800
cattgaacct caattgtgca tgcacactcc catacattgc accaatccat tcatgaacct 1860
gaagacaaat taaatggttt atttcagtaa aataaatgca agctaaagaa agctaaaaat 1920
aagctcattg ggttgaggtt gagctgaatt caactcgaat gtgctgactc gagctcgagt 1980
gagctggaaa cgagctaaat ggagctcaat gagaaggaat tgaactaatt cagctcgcat 2040
gggtttgcaa gcaatggtta gggagctggt caaaaagtgt gcccgaggcg tggtcgtatc 2100
acttatgttc actcattatt gatagtggta gttgctcaaa tgtggtgagt agctatttgg 2160
ttgattcttt aaagttacct tgcaccaaac accctaagcc atatcacctt cagtggctta 2220
atgaatgttc cgaagttaaa gttacgaaac agtccttggt cacttttaaa ctcgataatt 2280
atgaagatga agtatggtgt gacgtagtcc caatgcatgc ggcacatttg ctccttggac 2340
gaccttggca atttgatcgc gatgttacac accaaggtaa acttaatcga tactcccttg 2400
tatttaaagg taagaaattc acttttgctc ctttaaatcc tactgatgta tataaagatc 2460
aattgaggat gataaaattt tgtgaggggg taagggagaa agagcaagta caaaagacag 2520
agagaaaaga ggctaatagt agtggaaaaa gtcaaaattt aaaaagccca aaggtgagtg 2580
aatcctctag tggtaaaatg agcgaaaaaa atcttttggc aacaaaaaaa gatgtgaaga 2640
gagccctact taataaacag ccttgtatcc ttgtgaggtt taggcaaaat tatttatctt 2700
tatctaacat taacgaaaat tttcctagtg tgtttcaatc tcttttgcag gattatgagg 2760
atgtttttag tgaggcccct aaaggtttac cacctttacg agggatagaa catcaaattg 2820
acttaatacc cggagcttca atcccaaata gaccagccta tagatgcaat cctgaggaga 2880
caaaggaatt gcaaaggcaa gttgaggact tactagacaa agggtacatt cgtgagagcc 2940
taagtctttg cgctgttcct gtcttattgg tgccaaagaa agatggtacg ttttgtatgt 3000
gtgttgattg tcgcccaatc aacaagataa cggtaaagta tcgacaccca atccctagac 3060
ttgatgatat gcttgatgaa ttacatggtt caataatatt tacaaaaatt gatttaaaaa 3120
gcggttatca tcaaatttga atgagggaag gggatgaatg gaaaacagct tttaaaacaa 3180
aatttggatt gtatgaatgg ttagttatgc ctttcggcct 3220

To confirm that these sequences localize to cotton centromeres, PCR primers were generated for each of the CEN sequences shown in Table 10 (SEQ ID NOs:113, 116, 119, and 122), the PCR-amplified polynucleotides labeled with fluorescent dyes (such as ALEXA FLUOR® 488 (green) or ALEXA FLUOR® 568 (red)), and used in FISH to probe cotton root cells to ascertain the genomic localization of the probes. The PCR primers are shown in Table 12.

TABLE 12
Primers for amplifying exemplary CEN sequences
of SEQ ID NOs: 113, 116, 119, and 122

| SEQ | | |
|---|---|---|
| ID | | |
| NO | Sequence | Target |
| 114 | gcccatagtg ggggtttaat | SEQ ID NO: 113 Forward |
| 115 | cccttgtttg ctttgttggt | SEQ ID NO: 113 Reverse |
| 117 | ttcgacatgg gatgagttga | SEQ ID NO: 116 Forward |
| 118 | atcaaattgc caaggtcgtc | SEQ ID NO: 116 Reverse |
| 120 | tggttggtca catcaaggaa | SEQ ID NO: 119 Forward |
| 121 | gagtccgagt cgtttggttc | SEQ ID NO: 119 Reverse |
| 123 | gtaaaccgcc atcggaatta | SEQ ID NO: 122 Forward |
| | tgaa | |
| 124 | aggccgaaag gcataactaa | SEQ ID NO: 122 Reverse |
| | ccat | |

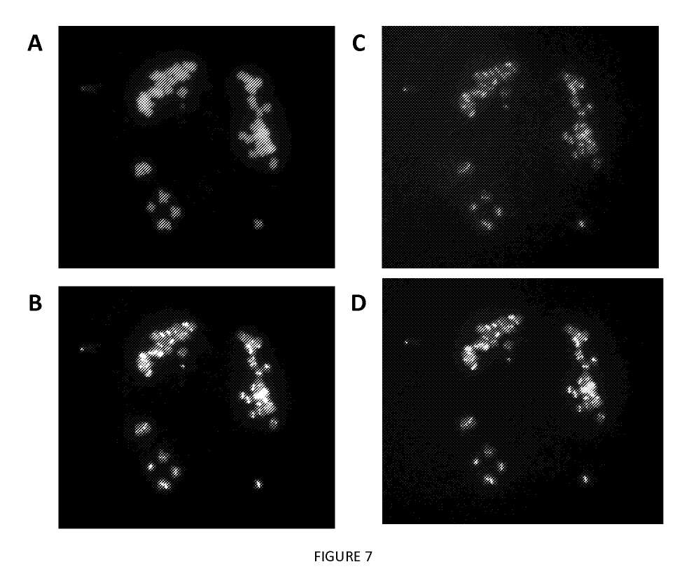

In a second approach, the investigators used PCR product of SEQ ID NO:97 to probe the BAC library described in Example 3. The investigators identified 46 BAC clones for further study, including characterizing these BACs for their genomic localization by FISH and BAC-end sequencing. Of these 46 BACs, 21 showed good centromere localization, localizing to centromeric constrictions in every chromosome in FISH preparations. FIG. 7 demonstrates this localization. In FIG. 7, the chosen BAC (“53H10”) was labeled with ALEXA FLUOR® 488 (green); the metaphase chromosomes were counter-stained with DAPI, and SEQ ID NO:96 was used as a probe dentify the centromeric constriction and was labeled with ALEXA FLUOR® 568 (red). As shown in FIG. 7A, all of the chromosomes counterstained with DAPI. FIG. 7B shows that BAC 53H10 appears to have labeled every chromosome in the preparation at centromeric constrictions. Confirming this observation that the BAC labeled centromeric constrictions, FIG. 7C shows the same preparation probed with SEQ ID NO:96, which stains centromeric constrictions, which gave an identical staining pattern as BAC 53H10. When the images are overlaid, there was no difference in staining, confirming that BAC 53H10 and SEQ ID NO:96 co-localize to centromeric constrictions (FIG. 7D).

### Example 8

### Assembly and Components of Cotton MCs (Prophetic)

Two methods have been developed to construct plant MCs. The first method relies on cre/lox recombination in that a BAC vector containing plant centromeric DNA and a loxP recombination site is recombined, by the action of cre re-combinase, with a donor vector carrying plant gene expression cassettes to generate a plant MC. The second method uses restriction enzyme digestion and ligation to produce (1) a vector fragment containing plant gene expression cassettes and (2) a centromere fragment with compatible cohesive ends. The two fragments are ligated into a circular structure to form a plant MC.

The components of the cotton MCs include a donor vector with a fluorescent (or other) reporter gene(s), a selectable maker gene, a cloning vector containing a cotton centromere sequence (selected from the group, for example, of SEQ ID NOs:90-92, 94-97), and can also contain telomere sequence. These components are described in detail below.

MC Construction by Cre-Lox Recombination

Cre recombinase-mediated exchange is used to construct MCs by combining the plant centromere fragments cloned in pBeIoBACI1 with a donor plasmid (e.g., pCH R487, Table 10). The recipient BAC vector carrying the plant centromere fragment contains a loxP recombination site; the donor plasmid contains two such sites, flanking the sequences to be inserted into the recipient BAC. MCs are constructed using a two-step method. First, the donor plasmid is linearized to allow free contact between the two loxP sites; in this step the backbone of the donor plasmid is eliminated. In the second step, the donor molecules are combined with centromere BACs and are treated with Cre recombinase, generating circular MCs with all the components of the donor and recipient DNA. MCs are delivered into E. coli and selected on medium containing antibiotics corresponding to the antibiotic resistance genes present on the vector molecule (e.g., kanamycin and chloramphenicol). Only vectors that successfully cre recombined and contained both selectable markers survive in the medium. MCs are then extracted from bacteria and restriction digested to verify DNA composition and calculate centromere insert size.

MC Construction by Restriction-Ligation

MCs are also constructed using standard cloning procedures. For example, a BAC containing a centromere fragment is digested with a restriction endonuclease that creates sticky ends, such as NotI. The digested DNA is then electrophoresed to purify the centromere fragment into a single band. The electrophoresis is carried out with conventional agarose gel electrophoresis with a linear electric field, or CHEF gel electrophoresis using an electric field that switches its orientation in the course of the run. When the electrophoresis is complete, the centromere fragment is visualized by ethidium bromide staining and illumination under ultraviolet light. The band corresponding to centromere DNA is then excised, and the DNA purified from the gel using conventional method for gel-purifying DNA fragments from agarose gels. The purified fragment is then ligated with a vector fragment that contains a low-copy E. coli backbone (e.g., the F′ plasmid replicon) and one or more plant-expressed genes. The vector fragment is digested with a restriction endonuclease leaving compatible sticky ends to those present on the centromere fragment. Alternatively, both fragments can be blunt.

To achieve a high rate of insertion of the centromere fragment into the vector, the phosphate groups are removed from the ends of the vector molecule by treating this DNA molecule with phosphatase; this step prevents ligation of the vector molecule to itself or to other vector molecules. After ligating vector DNA and centromere fragment, the MCs are delivered into E. coli and selected on medium containing antibiotics corresponding to the antibiotic-resistance genes present on the vector molecule (e.g., kanamycin and chloramphenicol). MCs are extracted from bacteria and restriction digested to verify DNA composition and calculate centromere insert size.

Cloning Vector

The vector, pBeIoBAC11 is an E. coli plasmid cloning vector based on the F′ plasmid replicon of E. coli. The vector contains a chloramphenicol resistance gene for selection of the plasmid in bacteria, repE, sopA/B/and C for maintenance of the plasmid in bacteria, and a LoxP recombination site for specific cleavage by Cre recombinase. A description of ail the genes contained within the vector and the location of the gene within the vector are set out in Table 13.

TABLE 13
pBeloBAC11 components

| Element | Size (bp) | Location (bp) | Details |
|---|---|---|---|
| Bacterial chloramplienicol | 660 | 766-1425 | Bacterial selectable marker |
| resistance | | (complementary) | |
| ori2 | 67 | 2370-2436 | F′ plasmid origin of replication from |
| | | | E. coli |
| repE | 755 | 2765-3520 | mediation of replication complex at Ori2 |
| | | | (Mori, Kondo et al. 1986) |
| SopA | 1166 | 410S-5274 | Partition of plasmid to bacterial |
| | | | daughter cells (Mori, Kondo et al. 1986) |
| SopB | 971 | 5274-6245 | Partition of plasmid to bacterial |
| | | | daughter cells (Mori, Kondo et al. 1986) |
| SopC | 474 | 6318-6791 | Partition of plasmid to bacterial |
| | | | daughter cells (Mori, Kondo et al. 1986) |
| cos | 400 | 7050-7449 | Lambda DNA recognition sequence for |
| | | | phage packaging |
| LoxP | 34 | 7467-7500 | Recombination site for Cre-mediated |
| | | | recombination (Abremski, Hoess et al. |
| | | | 1983) |

Source of Coding Regions Used in Plant-Expressed Genes

The fluorescent reporter genes DsRed and AMCyan were isolated from Athozoa species; and ZsYellow and ZsGrecn were isolated from Zoanthus sp (Matz, Fradkov et al. 1999). These reporter genes express proteins that are similar to Green Fluorescent Protein (GFP), that is a commonly used reporter gene in various biological systems, including plants. All fluorescent reporter genes can be commercially obtained. The selectable marker gene NPTII was isolated from E. coli (Bevan, Flavell et al. 1983).

Donor Vectors Used to Construct MCs Via Cre/Lox Recombinations pCHR487

The plasmid pCHR487 was developed using the commercially available high copy number E. coli cloning vector pUC19 (Yanisch-Perron, Vieira et al. 1985). The plasmid backbone was modified with the bacterial kanamycin selectable marker for maintenance of the plasmid in bacterial hosts, a pair of complementary loxP sites and a polylinker that facilitated the modular assembly of several plant-expressed genes for expression in plant MCs. Using standard cloning methods, plant-expressed gene cassettes were introduced into the modified pUC19 vector to construct pCHR487. This vector includes DsRed with a nuclear localization signal (Clontech), that was regulated by the Arabidopsis UBQ10 promoter (At4g05320) and the Arabidopsis pyruvate kinase terminator (At5g52920). The vector also includes the yeast TEF2 promoter from Saccharomyces cerevisiae and the plant kanamycin selectable marker Npt1 from E. coli. To enhance the stability of the Npt1 transcript, the Arabidopsis thaliana UBQ10 intron was inserted 5′ of the yeast TEF2 promoter and 3′ of the Npt1 gene. The UBQ10 intron is a naturally occurring component of the transcribed sequences from the Arabidopsis thaliana UBQ10 gene. The vector also contains a high-copy E. coli replication origin and an ampicillin bacterial selectable marker. MC genetic elements within the pCHR487 vector are set out in Table 14.

Prior to using pCHR4S7 to construct plant MCs, pCHR487 is digested with restriction endonucleases to linearize the plasmid and remove the high copy origin of replication and the bacterial ampicillin selectable marker, leaving loxP recombination sites on each end of the linear fragment. The resulting linearized vector is cre recombined in vitro to generate circular donor pCHR487 plasmids lacking a bacterial origin of replication and the ampicillin selectable marker. The donor pCHR487 construct is used to construct plant MCs.

TABLE 14
Donor Components of pCHR487

| Element | Size (bp) | Location (bp) | Details |
|---|---|---|---|
| UBQ10 promoter | 2038 | 361-2398 | Arabidopsis thaliana polyubiquitin |
| | | | promoter (At4g05320) |
| DsRed2 + NLS | 780 | 2435-3214 | Nuclear localized red fluorescent protein |
| | | | from Discosoma sp. (Matz, Fradkov et al. |
| | | | 1999) |
| Pyruvatc kinase | 332 | 3349-3680 | Arabidopsis thaliana pyruvate kinase |
| terminator | | | terminator (At5g52920) |
| Bacterial kanamycin | 817 | 3825-1641 | Bacterial kanamycin selectable marker |
| Act2 terminator | 800 | 4823-5622 | Arabidopsis thaliana Actin 2 terminator |
| NptII | 795 | 5685-6479 | Neomycin phosphotransferase II plant |
| | | | selectable marker |
| UBQ10 intron | 359 | 6507-6865 | PCR amplified Arabidopsis thaliana intron |
| | | | fromUBQ10 gene (At4g05320) for |
| | | | stabilization of NptII gene transcript and |
| | | | increase protein expression levels |
| TEF2 Promoter | 2000 | 6880-8879 | Saccharomyces cerevisiae translation |
| | | | elongation factor alpha promoter for |
| | | | expression of NptII |
| LoxP | 34 | 312-345 | Recombination site for Cre mediated |
| | | &8898-8931 | recombination (Abremski, Hoess et al. |
| | | | 1983) |

Cre recombinase-mediated exchange is used to construct MCs by combining the plant centromere fragments of pBeIoBACl 1 with the donor vector pCHR487. The recipient BAC vector carrying the plant centromere fragment contains a loxP recombination site that facilitates the introduction of donor DNA via the action of cre recombinase. Using purified cre recombinase in vitro, BAC centromere recipients are combined with donor pCHR487DNA, generating circular MCs with all the components of the donor and recipient DNA. MCs are delivered into E. coli and selected on medium containing kanamycin and chloramphenicol. Only vectors that successfully cre recombine contain both selectable markers and are easily selected from non-recombined events. MCs are then extracted from bacteria and restriction digested to verify DNA composition and calculate centromere insert size.

The pCHR488 donor vector also can also be used to construct MCs. In the pCHR488 MC donor vector, the yeast TEF2 promoter of pCHR487 is replaced with the yeast GPD1 promoter that drives the plant selectable marker NptII. The yeast GPD1 promoter was PCR amplified from Saccharomyces cerevisiae genomic DNA using standard PCR methods. Standard cloning methods were used to replace the TEF2 promoter and to insert the yeast GPD1 promoter. For construction of MCs, donor pCHR488 was generated as described for pCHR487. As with pCHR487, the circular donor pCHR488 lacks a bacterial origin of replication and the bacterial ampicillin selectable marker. The donor pCHR488 construct was used to construct plant MCs as described for pCHR487. MC genetic elements within the pCH R488 vector are set out in Table 15.

TABLE 15
Donor Components of pCHR488

| Element | Size (bp) | Location (bp) | Details |
|---|---|---|---|
| UBQ10 promoter | 2038 | 361-2398 | Arabidopsis thaliana polyubiquitin |
| | | | promoter (At4g05320) |
| DsRed2 + NLS | 780 | 2435-3214 | Nuclear localized red fluorescent protein |
| | | | from Discosoma sp. (Matz, Fradkov et al. |
| | | | 1999) |
| Pyruvate kinase | 332 | 3349-3680 | Arabidopsis thaliana pyruvate kinase |
| terminator | | | terminator (At5g52920) |
| Bacterial kanamycin | 817 | 3825-4641 | Bacterial kanamycin selectable marker |
| Act2 terminator | 800 | 4823-5622 | Arabidopsis thaliam Actin 2 terminator |
| NptII | 795 | 5685-6479 | Neomycin phosphotransterase II plant |
| | | | selectable marker |
| UBQ10 intron | 359 | 6500-6859 | PCR-amplified Arabidopsis thaliana intron |
| | | | from UBQ10 gene (At4g05320) for |
| | | | stabilization of NptII gene transcript and |
| | | | increase protein expression levels |
| GPD1 promoter | 2000 | 6880-8879 | Saccharomyces cerevisiae glycerol-3- |
| | | | phosphate dehydrogenase (NAD+) promoter |
| | | | for expression of NptII |
| LoxP | 34 | 312-345 & | Recombination site for Cre mediated |
| | | 8898-8931 | recombination (Abremski, Hoess et al. |
| | | | 1983) |

The pCHR489 donor vector aiso can also be used to construct MCs. In the pCHR489 MC donor vector, the yeast TEF2 promoter of pCHR487 is replaced with the Drosophila melanogaster Grim fly promoter for driving the plant selectable marker NptII. The Grim fly promoter was PCR amplified from Drosophila melanogaster genomic DNA using standard PCR methods. Standard cloning methods were used to replace the TEF2 promoter in pCHR487 with the Grim fly promoter to generate pCHR489. For construction of MCs, donor pCHR489 was generated as described for pCHR487. As with pCHR487, the circular donor pCHR489 lacks a bacterial origin of replication and the bacterial ampicillin selectable marker. The donor pCHR489 construct was used to construct plant MCs as described for pCHR4S7. MC genetic elements within the pCHR489 vector are set out in Table 16.

TABLE 16
Donor Components of pCHR489

| Genetic Element | Size (bp) | Location (bp) | Details |
|---|---|---|---|
| UBQ10 | 2038 | 361-2398 | Arabidopsis thaliana polyubiquitin |
| | | | promoter (At4g05320) |
| DsRed2 + NLS | 780 | 2435-3214 | Nuclear localized red fluorescent protein |
| | | | from Discosoma sp. (Matz, Fradkov et al. |
| | | | 1999) |
| Pyruvate kinase | 332 | 3349-3680 | Arabidopsis thaliana pyruvate kinase |
| terminator | | | terminator (At5g52920) |
| Bacterial kanamycin | 817 | 3825-4641 | Bacterial kanamycin selectable marker |
| Act2 terminator | 800 | 4823-5622 | Arabidopsis thaliana Actin 2 terminator |
| NptII | 795 | 5685-6479 | Neomycin phosphotransferase II plant |
| | | | selectable marker |
| UBQ10 intron | 359 | 6507-6865 | PCR-amplified Arabidopsis thaliana intron |
| | | | from UBQ10 gene (At4g05320) for |
| | | | stabilization of NptII gene transcript and |
| | | | increase protein expression levels |
| Grim Fly Promoter | 2191 | 6880-8879 | PCR-amplified promoter of grim (AKA |
| | | | BcDNA: RE28551) from Drosophila |
| | | | melanogaster |
| LoxP | 34 | 312-345 & | Recombination site for Cre mediated |
| | | 9081-9114 | recombination (Abremski, Hoess et al. |
| | | | 1983) |

Vectors Used to Construct MCs Via Standard Cloning Methods: pCHR510

As in pCHR487, pCHR510 contains DsRed with a nuclear localization signal and is regulated by the Arabidopsis UBQ10 promoter. The Arabidopsis pyruvate kinase terminator (At5g52920) was replaced by standard cloning procedures with the Arabidopsis thaliana triose phosphate isomerase terminator to prevent redundant use of the Arabidopsis pyruvate kinase terminator (At5g52920) in pCHR510. As in pCHR489, pCHR510 contains the plant selectable marker NptII regulated by the Drosophila melanogaster Grim fly promoter plus Arabidopsis UBQ10intron and the Arabidopsis Act2 terminator replaced with the Arabidopsis pyruvate kinase terminator (At5g52920). The vector also included a ZsGreen fluorescent gene (Clontech; Mountain View, Calif.) regulated by the Arabidopsis Act2 promoter plus naturally occurring intron and the Arabidopsis Act2 terminator. The high-copy E. coli backbone of pUC19 and the ampicillin bacterial selectable marker were replaced with the low copy pBeIoBAC11 backbone with the bacterial streptomycin resistance gene replacing the chloramphenicol resistance gene. An Arabidopsis thaliana ST11 sub-teloMCric fragment was introduced upstream of the Grim fly promoter to isolate the Grim fly promoter from possible promoter silencing when a centromere fragment was ligated into the donor vector. MC genetic elements within the pCHR510 vector are set out in Table 17 below.

TABLE 17
pCHR510 DNA donor components

| Element | Size (bp) | Location (bp) | Details |
|---|---|---|---|
| Bacterial | 10111 | 16912-17922 | Bacterial selectable marker |
| streptomycin | | | |
| resistance | | | |
| ori2 | 67 | 19158-19224 | F′ plasmid origin of replication from |
| | | | E. coli |
| repE | 755 | 19553-20308 | Mediation of replication complex at |
| | | | Ori2 (Mori, Kondo et al. 1986) |
| SopA | 1166 | 20896-22062 | Partition of plasmid to bacterial |
| | | | daughter cells (Mori, Kondo et al. |
| | | | 1986) |
| SopB | 971 | 22062-23033 | Partition of plasmid to bacterial |
| | | | daughter cells (Mori, Kondo et al. |
| | | | 1986) |
| SopC | 517 | 23106-23623 | Partition of plasmid to bacterial |
| | | | daughter cells (Mori, Kondo et al. |
| | | | 1986) |
| LoxP | 34 | 26-59 & | Recombination site for |
| | | 16212-16245 | Cre-mediated recombination |
| | | | (Abremski, Hoess et al. 1983) |
| ST11 subtelomeric | 4682 | 69-4750 | Arabidopsis thaliana subtelomeric |
| DNA | | (complementary) | DNA from chromosome 5 |
| Grim Promoter | 2187 | 4766-6956 | PCR-amplified Drosophila |
| | | | melanogasler Grim gene promoter |
| | | | for expression of NptII gene in |
| | | | plants |
| UBQ10 intron | 359 | 6963-7322 | PCR-amplified Arabidopsis thaliana |
| | | | intron from UBQ10 gene |
| | | | (At4g05320) for stabilization of |
| | | | NptII gene transcript and increase |
| | | | protein expression levels |
| NptII | 795 | 7350-8144 | Neomycin phosphorransferase II |
| | | | plant selectable marker |
| Pyruvate kinase | 332 | 8212-8543 | Arabidopsis thaliana pyruvate |
| terminator | | | kinase terminator (At5g52920) |
| Bacterial | 817 | 8731-9547 | Bacterial kanamycin selectable |
| kanamycin | | | marker |
| Act2 promoter + | 1482 | 9690-11171 | The Arabidopsis thaliana promoter |
| intron | | | Actin 2 plus natural intron |
| Zs Green | 695 | 11195-11890 | (Matz, Fradkov et al. 1999) |
| Act2 terminator | 800 | 11931-12730 | Arabidopsis thaliana Actin2 gene |
| | | | terminator |
| Triose phosphate | 450 | 12759-13208 | Arabidopsis thaliana triose |
| isomerase | | (complementary) | phosphate isomerase gene |
| | | | terminator |
| DsRed2 + NLS | 780 | 13343-14122 | Nuclear localized red fluorescent |
| | | (complementary) | protein from Discosoma sp. (Matz, |
| | | | Fradkov et al. 1999) |
| UBQ10 promoter | 2038 | 14159-16196 | Arabidopsis thaliana polyubiquitin |
| | | (complementary) | promoter (At4g05320) |

To construct MCs using pCHR510, the vector is linearized using standard restriction digestion procedures. The cotton centromere from selected cotton BACs, or comprising SEQ ID NOs:90-92, or SEQ ID NO:94, or SEQ ID NO:95, or SEQ ID NO:96, or SEQ ID NO:97 or previously selected cotton MCs is then restriction digested using NotI and ligated into pCHR510 using standard cloning procedures to generate MCs. MCs are delivered into E. coli and grown in selective medium. MCs are then extracted from bacteria and restriction digested to verify DNA composition and centromere insert size.

pCHR579

The pCHR579 MC donor vector was constructed using the same method to construct pCHR510, but without replacing the bacterial chloramphenicol gene in the low copy pBeIoBAC11 backbone. The pCHR579 vector is used to generate linear MCs by introducing plant telomere sequences into the MC. Using standard cloning methods the bacterial kanamycin gene was replaced with a bacterial kanamycin selectable marker surrounded by two plant telomere sequences and two unique I-Ppol homing endonuclease sequences.

Naturally occurring plant telomeres are composed of a seven-nucleotide repeat (taaaccc; SEQ ID NO:98). Plant telomeres were polymerized using standard PCR methods to generate approximately 800 base pair telomerc arrays. The telomere sequences were ligated using standard methods on both sides of the bacterial kanamycin gene. Two unique I-Ppol homing endonuclease restriction sites were introduced between each telomere and the kanamycin gene for linearization of the final MC construct.

MCs using pCHR579 are constructed as described for pCHR510 using cotton BAC or previously constructed MC centromeric DNA. MCs constructed from pCHR579 are linearized using restriction digested in vitro with homing endonuclease enzyme I-Ppol following standard restriction digest procedures. Linearization of the MC results in the removal of the bacterial kanamycin gene cassette leaving plant telomeres flanking both ends of the linear MC. Linear MCs are then ethanol-precipitated and used for plant transformation. MC genetic elements within the pCHR579 vector are set out in Table 18.

TABLE 18
pCHR579 DNA donor components

| Element | Size (bp) | Location (bp) | Details |
|---|---|---|---|
| Bacterial | 10111 | 16912-17922 | Bacterial selectable marker |
| streptomycin | | | |
| resistance | | | |
| ori2 | 67 | 19158-19224 | F′ plasmid origin of replication from |
| | | | E. coli |
| repE | 755 | 19553-20308 | Mediation of replication complex at |
| | | | Ori2 (Mori, Kondo et al. 1986) |
| SopA | 1166 | 20896-22062 | Partition of plasmid to bacterial |
| | | | daughter cells (Mori, Kondo et al. |
| | | | 1986) |
| SopB | 971 | 22062-23033 | Partition of plasmid to bacterial |
| | | | daughter cells (Mori, Kondo et al. |
| | | | 1986) |
| SopC | 517 | 23106-23623 | Partition of plasmid to bacterial |
| | | | daughter cells (Mori, Kondo et al. |
| | | | 1986) |
| LoxP | 34 | 26-59 & | Recombination site for |
| | | 16212-16245 | Cre-mediated recombination |
| | | | (Abremski, Hoess et al. 1983) |
| ST11 subtelomeric | 4682 | 69-4750 | Arabidopsis thaliana subtelomeric |
| DNA | | (complementary) | DNA from chromosome 5 |
| Grim Promoter | 2187 | 4766-6956 | PCR-amplified Drosophila |
| | | | melanogasler Grim gene promoter |
| | | | for expression of NptII gene in |
| | | | plants |
| UBQ10 intron | 359 | 6963-7322 | PCR-amplified Arabidopsis thaliana |
| | | | intron from UBQ10 gene |
| | | | (At4g05320) for stabilization of |
| | | | NptII gene transcript and increase |
| | | | protein expression levels |
| NptII | 795 | 7350-8144 | Neomycin phosphorransferase II |
| | | | plant selectable marker |
| Pyruvate kinase | 332 | 8212-8543 | Arabidopsis thaliana pyruvate |
| terminator | | | kinase terminator (At5g52920) |
| Plant telomere | 759 | 8598-9356 | based on plant consensus telomere |
| | | | sequence |
| Bacterial | 817 | 9532-10348 | Bacterial kanamycin selectable |
| kanamycin | | | marker |
| Plant telomere | 759 | 10482-11241 | Plant telomere PCR based on plant |
| | | | consensus telomere sequence |
| Acl2 promoter + | 1482 | 11287-12768 | Arabidopsis thaliana promoter Actin |
| intron | | | 2 plus natural intron |
| Zs Green | 695 | 12792-13487 | (Matz, Fradkov et al. 1999) |
| Act2 terminator | 800 | 13528-14327 | Arabidopsis thaliana Actin2 gene |
| | | | terminator. |
| Triose phosphate | 450 | 14356-14805 | Arabidopsis thaliana triose |
| isomerase | | (complementary) | phosphate isomerase gene |
| | | | terminator |
| DsRed2 + NLS | 780 | 14940-15719 | Nuclear localized red fluorescent |
| | | (complementary) | protein from Discosoma sp. |
| | | | (Matz, Fradkov et al. 1999) |
| UBQ10 promoter | 2038 | 15756-17793 | Arabidopsis thaliana |
| | | (complementary) | polyubiquitin promoter |
| | | | (At4g05320) |

Biolistic Particle Delivery of MCs

A biolistic delivery method using wet gold particles is used to transform cotton or other host plant cells. The method is adopted from the teachings of Mialhe and Miller (Mialhe and Miller 1994). To prepare the wet gold particles for bombardment, 1.0 μm gold particles is used and washed by mixing with 100% ethanol on a vortex followed by spinning the particles in a microfuge at 4000 rpm in order to remove supernatant. Subsequently, the gold particles are washed with sterile distilled water three times, followed by spinning in a microfuge to remove supernatant. The washed gold particles are resuspended in sterile distilled water at a final concentration of about 90 mg/ml and stored at 4° C. For bombardment, the gold particle suspension (90 mg/ml) is mixed rapidly with about 1 μg/μl DNA solution (in dH₂O or TE), 2.5M CaCl₂, and 1M spermidine.

To prepare explant tissues for DNA delivery: three days prior to bombardment, an internode of the cotton plant is cut. The internode explant is cut longitudinally with a scalpel to cut a thin slice (⅙-¼ of the intcrnode) off one side of the explant. The prepared inlernodes are placed wound side down on Petri dishes with regeneration media. The Petri dishes are wrapped with tape and placed wound side up under light. The explants are grown for 3 days prior to bombardment.

For bombardment of cotton suspension cells, a method as follows can be used. Cells are harvested by centrifugation (1200 rpm for 2 minutes) on the day of bombardment. The cells are plated onto 50 mm circular polyester screen cloth disks placed on petri plates with solid medium. The solid medium used is the same medium that the cells are grown in and can contain MS salts, Gamborg's vitamins, sucrose, 2,4D (2,4-dichlorophenoxyacetic acid), MES pH 5.8+(solid medium only), plus 0.26% gelrite, or 0.6% tissue culture agar, added before autoclaving. Approximately 1.5 ml packed cells are placed on each filter disk, and dispersed uniformly into a very even spot approximately 1 inch in diameter.

Bombardment of the cells is carried out in the Bio-Rad PDS-1000/He Biolistic Particle Delivery System (Bio-Rad). The DNA/gold suspension is resuspended and immediately inserted onto the grid of the filter holder. A 50 mm circular polyester screen cloth disk containing the cells is laced into a fresh 60 mm petri dish and the cells are covered with a 10×10 cm square of sterile nylon or Dacron chiffon netting. The metal cylinder is inserted into the petri dish and used to push the netting down to the bottom of the dish. This weight prevents the cells from being dislodged from the plate during bombardment. The petri dish containing the cells is then placed onto the sample holder, and positioned in the sample chamber of the gene gun and bombarded with the DNA/gold suspension. After the bombardment, the cells are scraped off the filter circle in the petri dish containing solid medium with a sterile spatula and transferred to fresh medium in a 125 ml glass bottle. The bottles are transferred onto a shaker and grown while shaking at 150 rpm.

A biolistic delivery method using dry gold particles can also be carried out to deliver MCs to cotton cells. A method as follows can be used. 1.0 or 0.6 μm gold particles are washed in 70% ethanol with vigorous shaking on a vortex for 3 to 5 minutes, followed by soaking in 70% ethanol for 15 minutes. The gold particles are then spun in a microfuge to remove the supernatant and washed three times in sterile distilled water. The gold particles are suspended in 50% glycerol at a concentration of 60 mg/ml and stored at 4° C. For bombardment, the dry gold particles are resuspended using a vortex for 5 minutes to disrupt agglomerated particles. Subsequently, the dry gold particles are mixed rapidly with DNA, 2.5M CaCl₂ and 0.2M spermidine in a siliconized, sterile microfuge tube. The samples are allowed to settle for 1 minute and then are spun in a microfuge for 10 seconds to remove the supernatant. Subsequently, the DNA/gold particles are washed once with 70% ethanol, followed by two washed in 100% ethanol. A portion of the DNA/gold mixture is evenly placed on a macrocarrier. The macrocarrier is then placed in the Bio-Rad PDS-1000/He Biolistic Particle Delivery System, and the bombardment is done at rupture disk pressures ranging from 450 psi to 2,200 psi.

### Example 8

### Selection of Cotton Cell Clones Stably Containing MC DNA (Prophetic)

Use of Visible Marker Genes

The presence of visible marker genes allows for visual selection of cotton cells stably containing MC DNA because any adchromosomal cells or cell clusters are readily identified by virtue of fluorescent protein expression.

Transient assays are used to test MCs for their ability to become established in cells following DNA delivery, and for their ability to be inherited in mitotic cell divisions. Expression of a visible marker encoded by a gene present on the MC, such as a fluorescent protein gene, can be used to detect MC presence in the cell, and to follow mitotic inheritance of the MC. In this assay, MCs are delivered to cotton cells of a population that is undergoing cell division.

After DNA delivery, the cell population is monitored for fluorescent protein expression over the course of one to several weeks. MCs containing active centromeres are observed through the formation of fluorescent cell clusters that are derived from a single progenitor cell that divided and passed the MCs to its daughter cells. Accordingly, single fluorescent cells and clusters of fluorescent cells of various sizes are scored in the growing cell population after DNA delivery.

Manipulation of Adchromosomal Tissue to Homogeneity

After identifying clusters of fluorescent cells in bombarded suspension ceil cultures, physical manipulations are carried out to allow for the preferential expansion of cells harboring the delivered genes. Non-fluorescent tissue surrounding the fluorescent clusters is trimmed to avoid overgrowth of fluorescent cells by non-fluorescent ones, while retaining a minimum tissue size capable of rapid growth. These manipulations are performed under sterile conditions with the use of a fluorescence stereomicroscope allowing for visualization of the fluorescent cells and cell clumps in the larger pieces of tissue. In between the mechanical purification steps, the tissue is allowed to grow on appropriate media, either in the presence or absence of selection. Over time, a pure population of fluorescent cells should result.

### Example 9

### Regeneration of Cotton Plants from Adchromosomal Cell Clones (Prophetic)

Cotton MCs are used in stable transformation to successfully regenerate adchromosomal cotton plants. The cotton MCs represent candidate cotton centromere sequences for the delivery and transmission of stable cotton MCs.

While the invention is susceptible of embodiment in many different forms, specific embodiments thereof with the understanding that the present disclosure is to be considered as an exemplification of the principles of the invention and is not intended lo limit the invention to the specific embodiments illustrated. It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims. All cited references are incorporated by reference herein as if set forth specifically in this specification.

### CITED NON-PATENT LITERATURE

Abel, P. P., R. S, Nelson, et al. (1986). “Delay of disease development in transgenic plants that express the tobacco mosaic virus coat protein gene.” Science 232(4751): 738-743.
Abremski, K., R. Hoess, et al. (1983). “Studies on the properties of P1 site-specific recombination: evidence for topologically unlinked products following recombination.” Cell 32(4): 1301-1311.
Altschul, S. F., W. Gish, et al. (1990). “Basic local alignment search tool.” J Mol Biol 215(3): 403-410.
Aoyama, T. and N. H. Chua (1997). “A glucocorticoid-mediated transcriptional induction system in transgenic plants.” Plant J 11(3): 605-612.
Ausubel, F. M. (1987). Current protocols in molecular biology. Brooklyn, N.Y.
Media, Pa., Greene Publishing Associates; J. Wiley, order fulfillment.
Benson, G. (1999). “Tandem repeats finder: a program to analyze DNA sequences.” Nucleic Acids Res 27(2): 573-580.
Bevan, M. W., R. B. Flavell, et al. (1983). “A chimaeric antibiotic resistance gene as a selectable marker for plant cell transformation.” Nature 304(5922): 184-187.
Bonfield, J. K. and R. Staden (1995). “The application of numerical estimates of base calling accuracy to DNA sequencing projects.” Nucleic Acids Res 23(8): 1406-1410.
Bowler, C., M. Montagu, et al. (1982). “Superoxide dismutase and stress tolerance.” Annu Rev Plant Physiol Plant Mol Biol 43: 83-116.
Callis, J., M. Fromm, et al. (1987). “Introns increase gene expression in cultured maize cells.” Genes Dev 1(10): 1183-1200.
Carlson, S. R., G. W. Rudgers, et al. (2007). “Meiotic transmission of an in vitro-assembled autonomous maize minichromosome.” PLoS Genet. 3(10): 1965-1974.
Cavaliere, F. M., G. L. Scoarughi, et al. (2009). “Interspecific transfer of mammalian artificial chromosomes between farm animals.” Chromosome Res 17(4): 507-517.
Cheng, Z., F. Dong, et al. (2002). “Functional rice centromeres are marked by a satellite repeat and a centromere-specific retrotransposon.” Plant Cell 14(8): 1691-1704.
Chowrira, B. M., P. A. Pavco, et al. (1994). “In vitro and in vivo comparison of hammerhead, hairpin, and hepatitis delta virus self-processing ribozyme cassettes.” J Biol Chem 269(41): 25856-25864.
Chrispeels, M. J., D. E. Sadava, et al. (2003). Plants, genes, and crop biotechnology. Boston, Jones and Bartlett Publisher.
Cottarel, G., J. H. Shero, et al. (1989). “A 125-base-pair CEN6 DNA fragment is sufficient for complete meiotic and mitotic centromere functions in Saccharomyces cerevisiae.” Mol Cell Biol 9(8): 3342-3349.
Czapla, T. and B. Lang (1991). J. Econ. Entomol. 83: 2480-2485.
de la Luz Gutierrez-Nava, M., M. J. Aukerman, et al. (2008). “Artificial trans-acting siRNAs confer consistent and effective gene silencing.” Plant Physiol 147(2): 543-551.
Dunwell, J. M. (1999). “Transformation of maize using silicon carbide whiskers.” Methods Mol Biol 111: 375-382.
Fitzpatrick (1989). “Pleiotropic gene found in barley plant.” Gen. Engineering News 22: 7.
Gatehouse, A., F. Dewey, et al. (1984). J. Sci. Food. Agric. 35: 373-380.
Guerrero, F. D., J. T. Jones, et al. (1990). “Turgor-responsive gene transcription and RNA levels increase rapidly when pea shoots are wilted. Sequence and expression of three inducible genes.” Plant Mol Bio115(1): 11-26.
Hall, A. E., A. Fiebig, et al. (2002). “Beyond the Arabidopsis genome: opportunities for comparative genomics.” Plant Physiol 129(4): 1439-1447.
Hammock, B. D., B. C. Bonning, et al. (1990). “Expression and effects of the juvenile hormone esterase in a baculovirus vector.” Nature 344(6265): 458-461.
Haseloff, J. and W. L. Gerlach (1988). “Simple RNA enzymes with new and highly specific endoribonuclease activities.” Nature 334(6183): 585-591.
Hawkins, J. S., H. Kim, et al. (2006). “Differential lineage-specific amplification of transposable elements is responsible for genome size variation in Gossypium.” Genome Res 16(10): 1252-1261.
Hilder, V. A., A. M. R. Gatehouse, et al. (1987). “A novel mechanism of insect resistance engineered into tobacco.” Nature 330(6144): 160-163.
Kato, A., J. C. Lamb, et al. (2004). “Chromosome painting using repetitive DNA sequences as probes for somatic chromosome identification in maize.” Proc Natl Acad Sci USA 101(37): 13554-13559.
Lee, C. A. and M. H. Saier, Jr. (1983). “Use of cloned mtI genes of Escherichia coli to introduce mtI deletion mutations into the chromosome.” J Bacteriol 153(2): 685-692.
Levings, C. S., 3rd (1990). “The Texas Cytoplasm of Maize: Cytoplasmic Male Sterility and Disease Susceptibility.” Science 250(4983): 942-947.
Liu, L., W. Guo, et al. (2003). “Inheritance and fine mapping of fertility restoration for cytoplasmic male sterility in Gossypium hirsutum L.” Theor Appl Genet. 106(3): 461-469.
Loomis, S., J. Carpenter, et al. (1989). “Cryoprotective capacity of end-products of anaerobic metabolism.” J. Exp. Zool. 252: 9-15.
Lorence, A. and R. Verpoorte (2004). “Gene transfer and expression in plants.” Methods Mol Biol 267: 329-350.
Luo, S., A. E. Hall, et al. (2004). “Whole-genome fractionation rapidly purifies DNA from centromeric regions.” Nat Methods 1(1): 67-71.
Ma, J. and S. A. Jackson (2006). “Retrotransposon accumulation and satellite amplification mediated by segmental duplication facilitate centromere expansion in rice.” Genome Res 16(2): 251-259.
Mariani, C., M. D. Beuckeleer, et al. (1990). “Induction of male sterility in plants by a chimaeric ribonuclease gene.” Nature 347(6295): 737-741.
Matz, M. V., A. F. Fradkov, et al. (1999). “Fluorescent proteins from nonbioluminescent Anthozoa species.” Nat Biotechnol 17(10): 969-973.
Mialhe, E. and L. H. Miller (1994). “Biolistic techniques for transfection of mosquito embryos (Anopheles gambiae).” Biotechniques 16(5): 924-931.
Mori, H., A. Kondo, et al. (1986). “Structure and function of the F plasmid genes essential for partitioning.” J Mol Biol 192(1): 1-15.
Murdock, L., J. Huesing, et al. (1990). “Biological effects of plant lectins on the cowpea weevil.” Phytochemistry 29: 85-89.
Needleman, S. B. and C. D. Wunsch (1970). “A general method applicable to the search for similarities in the amino acid sequence of two proteins.” J Mol Biol 48(3): 443-453.
Phelps-Durr, T. L. and J. A. Birchler (2004). “An asymptotic determination of minimum centromere size for the maize B chromosome.” Cytogenet Genome Res 106(2-4): 309-313.
SanMiguel, P., B. S. Gaut, et al. (1998). “The paleontology of intergene retrotransposons of maize.” Nat Genet. 20(1): 43-45.
Schwab, R., S. Ossowski, et al. (2006). “Highly specific gene silencing by artificial microRNAs in Arabidopsis.” Plant Cell 18(5): 1121-1133.
Sun, X., J. Wahlstrom, et al. (1997). “Molecular structure of a functional Drosophila centromere.” Cell 91(7): 1007-1019.
Symons, R. H. (1992). “Small catalytic RNAs.” Annu Rev Biochem 61: 641-671.
Unger, E. A., J. M. Hand, et al. (1989). “Isolation of a cDNA encoding mitochondrial citrate synthase from Arabidopsis thaliana.” Plant Mol Biol 13(4): 411-418.
Watrud (1985). Engineered Organism in the Environment: Scientific Issues. H. e. al., American Society of Microbiology Washington D.C.
Wendel, J. and R. Cronn (2002). “Polyploidy and the evolutionary history of cotton.” Adv Agron 78: 139-186.
Wolter, F. P., R. Schmidt, et al. (1992). “Chilling sensitivity of Arabidopsis thaliana with genetically engineered membrane lipids.” Embo J 11(13): 4685-4692.
Xu, D., X. Duan, et al. (1996). “Expression of a Late Embryogenesis Abundant Protein Gene, HVA1, from Barley Confers Tolerance to Water Deficit and Salt Stress in Transgenic Rice.” Plant Physiol 110(1): 249-257.
Yamaguchi-Shinozaki, K., M. Kozumi, et al. (1992). “Molecular cloning and characterization of 9 cDNAs for genes that are responsive to desiccation in Arubidopsis thaliunn: sequence analysis of one cDNA clone that encodes a putative transmembrane channel protein.” Plant Cell Physiol. 33: 217-224.
Yanisch-Perron, C., J. Vieira, et al. (1985). “Improved M13 phage cloning vectors and host strains: nucleotide sequences of the M13mp 18 and pUC19 vectors.” Gene 33(1): 103-119.
Zhao, X., R. A. Wing, et al. (1995). “Cloning and characterization of the majority of repetitive DNA in cotton (Gossypium L.).” Genome 38(6): 1177-1188.
Zhao, X. P., Y. Si, et al. (1998). “Dispersed repetitive DNA has spread to new genomes since polyploid formation in cotton.” Genome Res 8(5): 479-492.
Zuo, J., Q. W. Niu, et al. (2002). “The WUSCHEL gene promotes vegetative-to-embryonic transition in Arabidopsis.” Plant J 30(3): 349-359.

1-19. (canceled)

20. An isolated polynucleotide comprising a nucleic acid sequence having at least 70% sequence identity with SEQ ID NO:94, or fragment thereof, wherein the polynucleotide confers an ability to the polynucleotide to be transmitted through mitosis or meiosis in a plant cell with a transmission efficiency greater than 50%.

21-26. (canceled)

27. A recombinant DNA construct comprising the polynucleotide of claim 20.

28. (canceled)

29. The polynucleotide of claim 20, wherein the nucleic acid sequence having at least 70% sequence identity to the polynucleotide is selected from the group consisting of SEQ ID NO: 95, 96, 97, 113, 116, 119 and 122.

30. The polynucleotide of claim 20, wherein the polynucleotide consists of a nucleic acid sequence of SEQ ID NO:97.

31. A plant cell comprising the recombinant DNA construct of claim 27.

32-37. (canceled)

38. A material comprising cotton produced by a plant comprising a plant cell of claim of claim 31.

39. (canceled)

40. An isolated polynucleotide comprising a copy of a nucleic acid sequence having at least 70% sequence identity to SEQ ID NO:95, or a fragment thereof and two copies of a nucleic acid sequence having at least 70% sequence identity to SEQ ID NO:96, or a fragment thereof, wherein a first copy is located 5′ of SEQ ID NO:95, and the second copy of SEQ ID NO:96 is located 3′ of SEQ ID NO:95, wherein the polynucleotide confers an ability to the polynucleotide to be transmitted through mitosis or meiosis in a plant cell with a transmission efficiency greater than 50%.

41-50. (canceled)

51. A recombinant DNA construct comprising the polynucleotide of claim 40.

52. (canceled)

53. A plant cell comprising the DNA construct of claim 51.

54-59. (canceled)

60. A material comprising cotton produced by a plant comprising a plant cell of claim of claim 55.

61. (canceled)

62. A polynucleotide comprising a structure comprising a core sequence flanked by two retroelement-related sequences, wherein the polynucleotide confers an ability to the polynucleotide to be transmitted through mitosis or meiosis in a plant cell with a transmission efficiency greater than 50%.

63. The polynucleotide of claim 62, wherein the retroelement-related sequences are at least 90% identical to each other.

64. (canceled)

65. The polynucleotide of claim 62, wherein at least one of the two retroelement-related sequences has at least 70% sequence identity with SEQ ID NO:96.

66-69. (canceled)

70. The polynucleotide of claim 62, wherein the core sequence has at least 70% sequence identity to SEQ ID NO:95.

71-75. (canceled)

76. A recombinant DNA construct comprising the polynucleotide of claim 62.

77. (canceled)

78. A plant cell comprising the recombinant DNA construct of claim 76.

79-84. (canceled)

85. A material comprising cotton produced by a plant comprising a plant cell of claim of claim 78.

86-117. (canceled)

118. A method for identifying a centromere sequence in a plant, comprising:
(a). obtaining genomic DNA sequences for at least a portion of the plant's genome;
(b). assembling the DNA sequences into low-stringency contigs;
(c). selecting those contigs having deep reads;
(d). analyzing the deep read contigs for the presence of tandem repeats;
(e). eliminating those contigs having tandem repeats from the contigs having deep reads;
(f). determining a consensus sequence for each remaining contig having a deep read;
(g). designing and making a nucleic acid probe based on the consensus sequence;
(h). using the probe in an assay to detect centromere sequences.

119-120. (canceled)

121. A centromere sequence identified by the method of claim 118.

122. A minichromosome made by the method of claim 118.
